(19) United States
(12) Patent Application Publication
(10) Pub. No.: US 2008/0301822 A1
(43) Pub. Date: Dec. 4, 2008

(54) Role of alpha1-adrenergic receptors

(75) Inventor: Dianne M. Perez, Broadview Heights, OH (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: The Cleveland Clinic Foundation, Cleveland, OH (US)

(21) Appl. No.: 12/151,594

(22) Filed: May 7, 2008

Related U.S. Application Data
(63) Continuation of application No. PCT/US06/43015, filed on Nov. 3, 2006.
(60) Provisional application No. 60/734,076, filed on Nov. 7, 2005.

(51) Int. Cl. A01K 67/027; C12N 15/09; C07H 21/04; C12N 5/06; G01N 33/53

(52) U.S. Cl. 800/3; 800/18; 800/25; 536/23.1; 435/354; 435/7.1

(57) ABSTRACT

The present invention is directed to a transgenic non-human mammal (e.g., a rodent such as a mouse) whose genome comprises a recombinant nucleic acid sequence comprising an α_{1A}-adrenergic receptor (AR) and a marker peptide (e.g., a fluorescent peptide such as green fluorescent protein and an enhanced green fluorescent protein) operably linked to all or a functional portion of an α_{1A}-AR promoter, wherein the α_{1A}-AR (e.g., human α_{1A}-AR) and the marker peptide are expressed as a fusion protein in the transgenic non-human mammal. The present invention also provides methods of producing a transgenic non-human mammal whose genome comprises a recombinant nucleic acid sequence comprising an α_{1A}-AR and a marker peptide, as well as targeting constructs for use in such methods. The invention also provides a source of cells (for example, tissue, cells, cellular extracts, organelles) and animals useful for elucidating the function of α_{1A}-AR in intact animals. Further aspects of the invention provide methods for the identification of agents that modulate neural stem cell or progenitor cell differentiation by α_{1A}-AR; methods of determining whether a cell is a neural stem cell; methods of regulating differentiation or proliferation of a neural stem cell or progenitor cell; and methods of treating neurodegenerative diseases, cognitive impairment or conditions.

### RELATED APPLICATION

This application is a continuation of International Application No. PCT/US2006/043015 which designated the United States and was filed on Nov. 3, 2006, published in English, claims the benefit of U.S. Provisional Application No. 60/734,076, filed on Nov. 7, 2005. The entire teachings of the above application are incorporated herein by reference.

### BACKGROUND OF THE INVENTION

Adrenergic receptors (ARs) are members of the G-protein-coupled receptor (GPCR) superfamily of cell surface membrane proteins that mediate the sympathetic nervous system via the effects of catecholamines, norepinephrine and epinephrine. The three α₁-AR subtypes (α_{1A}, α_{1B}, α_{1D}) have been cloned and characterized (Cotecchia et al, Proc. Natl. Acad. Sci., 85:7159-7163 (1988); Perez et al., Mol. Pharmacol., 40: 876-883 (1991); Perez et al., Mol. Pharmacol., 46:823-831 (1994)). Of the sympathetically innervated tissues, the cardiovasculature is the most characterized. α₁-AR stimulation has a major role in the contraction and growth of vascular smooth muscle cells and the regulation of blood pressure (reviewed in Piascik and Perez, J. Pharmacol. Exp. Ther., 298:403-410 (2001)). These responses are transduced primarily via receptor coupling to the G_{q} pathway and the resulting calcium release.

α₁-ARs are abundant receptors in the brain. Of known functions, α₁-AR activation can increase the excitation of glutamate in the cerebral cortex (Mouradian et al., Brain Res., 546:83-95 (1991) and enhance neurotransmitter release from glutamate terminals in the neocortex (Marek and Aghajanian, Eur. J. Pharmacol., 367:197-206 (1999)). They may also play a role in attention and memory (Sirvia and MacDonald, Pharmacol. Ther., 83:49-65 (1999)). The mechanisms underlying the function of norepinephrine in memory processing could be by modulating the efficacy of glutamate synaptic transmission via activation of α₁-ARs (Scheiderer et al., J. Neuorphysiol., 91:1071-1077 (2004)).

Previous studies on α₁-AR localization that used radioactive in situ hybridization or autoradiography were not resolving enough to determine cell type localization (Jones et al., J. Comp. Neurol., 231:190-208 (1985); Palacios et al. Brain Res., 419:65-75 (1987); Zilles et al. Neurosci., 40:307-320, (1991); McCune et al., Neurosci., 57:143-151 (1993); Pieribone et al., J. Neurosci., 14:4252-4268 (1994); Nicholas et al., Trends Pharmacol. Sc., 17:245-255 (1996); Domyancic and Morilak, J. Comp. Neurol., 386:358-378 (1997)). In addition, the protein translated from the mRNA can be transported to different parts of the cell. While immunohistochemistry would provide accurate localization of the receptor protein, the lack of high avidity antibodies and selective antagonists against the α₁-AR subtypes has hampered localization and functional studies. Antibodies currently available for α₁-ARs are useless for studies using native tissues.

Thus, signal transduction by α₁-ARs is involved in a variety of responses such as neurotransmission and sympathetic control of various organ systems. These receptors are a current therapeutic target in the management of hypertension through their role in smooth muscle contraction, but their role in the central nervous system (CNS) is not understood very well and tools (antibodies, selective ligands) to study these receptors are not available for their use in tissues.

### SUMMARY OF THE INVENTION

As described herein, transgenic mice that endogenously overexpress receptor-Enhanced Green Fluorescent Protein (EGFP) tagged forms of the α_{1A}-AR subtype have been produced. Antibodies against α_{1A}-AR have been categorically rejected due to low avidity. However, as shown herein, these receptors can be identified in the CNS through our transgenic EGFP model systems. α_{1A}-ARs are located on neurons, GABAergic interneurons and are expressed on NG2-positive oligodendrocyte progenitors but are not expressed in mature oligodendrocytes or astrocytes, and because of this, are likely a switch involved in glial maturation. It is shown herein that α_{1A}-ARs are likely neurogenic receptors, being expressed in adult cells in the subventricular zone (SVZ) that are Notch-1, nestin and vimentin positive. However, not all notch-1 or nestin-positive cells express the α_{1A}-AR and not all α_{1A}-AR cells express nestin or notch-1 in the SVZ. Some of these α_{1A}-AR positive cells are also D1x2 positive, indicating that they are transiently amplifying progenitors (TAP) cells and form neuroblasts; but not all nestin-positive, α_{1A}-AR positive cells in the SVZ are D1x2-positive. This differential labeling by Notch-1, Nestin and D1x2 indicates that α_{1A}-ARs are expressed on various cell populations that are either stem cells and/or progenitors. The α_{1A}-AR agonist phenylephrine differentiates EGF-responsive neurospheres isolated from normal mice into all three cell types (neurons, astrocytes and NG2 oligodendrocyte progenitors) but favors the differentiation of neurons and NG2 progenitors, exactly as the α₁-AR cell types found throughout the adult brain (Papay, R., et al., J. Comparative Neurol., 248:1-10 (2004). The results described herein indicate that the α_{1A}-AR is being expressed and regulates the differentiation of neural stem cells, TAP cells, neuroblasts and oligodendrocyte progenitors. The α_{1A}-AR is likely involved in the differentiation process that commits the differentiation into neurons and NG2 precursors but turns off the differentiation of astrocytes and mature oligodendrocytes (FIG. 7).

Accordingly, the present invention is directed to a transgenic non-human mammal (e.g., a rodent such as a mouse) whose genome comprises a recombinant nucleic acid sequence comprising an α_{1A}-adrenergic receptor (AR) and a marker peptide (e.g., a fluorescent peptide such as green fluorescent protein and an enhanced green fluorescent protein) operably linked to all or a functional portion of an α_{1A}-AR promoter, wherein the α_{1A}-AR (e.g., human α_{1A}-AR) and the marker peptide are expressed as a fusion protein in the transgenic non-human mammal. The recombinant nucleic acid sequence can further comprise a promoter (e.g., a mouse, rat, hamster, feline, canine, primate (human) α_{1A}-AR promoter) that directs expression of the fusion protein. In a particular embodiment, the promoter directs overexpression of the fusion protein in the transgenic non-human mammal. The marker peptide can be fused to a terminus (e.g., the N-terminus; the C-terminus) of the α_{1A}-AR.

In a particular embodiment, the invention is directed to a transgenic mouse whose genome comprises a recombinant nucleic acid sequence which comprises a mouse α_{1A}-adrenergic receptor (AR) promoter operably linked to a human α_{1A}-AR and an enhanced green fluorescent protein (EGFP), wherein the human α_{1A}-AR and the EGFP are expressed as a fusion protein in the transgenic mouse and the EGFP is fused to the C-terminus of the human α_{1A}-AR.

In yet another embodiment, the genome of the transgenic non-human mammal comprises a recombinant nucleic acid sequence comprising a marker protein, under the control of all or a functional portion of an α_{1A}-AR promoter.

Also encompassed by the present invention is a transgenic non-human mammal (e.g., mouse) whose genome comprises a recombinant nucleic acid sequence which comprises an α_{1A}-adrenergic receptor (AR) promoter (e.g., mouse) operably linked to a marker peptide (e.g., an enhanced green fluorescent protein (EGFP)).

The present invention is also directed to a method of producing the transgenic non-human mammals described herein. In one embodiment, the present invention is directed to a method of producing a transgenic non-human mammal whose genome comprises a recombinant nucleic acid sequence comprising an α_{1A}-AR and a marker peptide, wherein the α_{1A}-AR and the marker peptide are expressed as a fusion protein in the transgenic non-human mammal. The method comprises introducing a targeting construct which comprises the recombinant nucleic acid sequence comprising the α_{1A}-AR and the marker peptide, wherein the α_{1A}-AR and the marker peptide are expressed as a fusion protein, into a pronuclei of an embryo. The embryo can then be introduced into a pseudo-pregnant non-human female mammal under conditions in which the non-human female mammal gives birth to a chimeric transgenic non-human mammal whose genome comprises the recombinant nucleic acid sequence comprising the α_{1A}-AR and the marker peptide. The chimeric transgenic non-human mammal is bred with a second mammal to generate heterozygous F1 progeny that are heterozygous for the recombinant nucleic acid sequence comprising the α_{1A}-AR and the marker peptide; and the heterozygous F1 progeny are crossbred under conditions in which a transgenic non-human mammal whose genome comprises a recombinant nucleic acid sequence comprising an α_{1A}-adrenergic receptor (AR) and a marker peptide, wherein the α_{1A}-AR and the marker peptide are expressed as a fusion protein in the transgenic non-human mammal, and homozygote F2 progeny is produced.

In another embodiment, the present invention is directed to a method of producing a transgenic non-human mammal whose genome comprises a recombinant nucleic acid sequence comprising an α_{1A}-AR promoter and a marker peptide, wherein the marker peptide is expressed under the control of the α_{1A}-AR promoter (operably linked) in the transgenic non-human mammal. The method comprises introducing a targeting construct which comprises the recombinant nucleic acid sequence comprising the α_{1A}-AR promoter and the marker peptide, into a pronuclei of an embryo. The embryo can then be introduced into a pseudo-pregnant non-human female mammal under conditions in which the non-human female mammal gives birth to a chimeric transgenic non-human mammal whose genome comprises the recombinant nucleic acid sequence comprising the α_{1A}-AR promoter and the marker peptide. The chimeric transgenic non-human mammal is bred with a second mammal to generate heterozygous F1 progeny that are heterozygous for the recombinant nucleic acid sequence comprising the α_{1A}-AR promoter and the marker peptide; and the heterozygous F1 progeny are crossbred under conditions in which a transgenic non-human mammal whose genome comprises a recombinant nucleic acid sequence comprising an α_{1A}-adrenergic receptor (AR) promoter and a marker peptide, wherein the marker peptide is expressed under the control of the α_{1A}-AR promoter in the transgenic non-human mammal, and homozygote F2 progeny is produced.

The invention also encompasses a transgenic non-human mammal produced by the method described above.

Also provided are targeting constructs comprising all or a functional portion of an α_{1A}-AR promoter sequence, a marker peptide and/or all or a functional portion of α_{1A}-AR sequence, wherein the marker peptide and/or the α_{1A}-AR sequence are expressed under the control of (operably linked to) the α_{1A}-AR promoter sequence. In one embodiment, the construct comprises in a 5′ to 3′ direction about a 4.4 kb fragment of an α_{1A}-AR promoter sequence, an α_{1A}-AR sequence and an enhanced green fluorescent protein sequence. In another embodiment, the construct comprises in a 5′ to 3′ direction about a 4.4 kb fragment of an α_{1A}-AR promoter sequence and an enhanced green fluorescent protein sequence.

The present invention is also directed to an isolated cell or cell line whose genome comprises a recombinant nucleic acid sequence comprising an α_{1A}-adrenergic receptor (AR) and a marker peptide, wherein the α_{1A}-AR and the marker peptide are expressed as a fusion protein in the cell.

Also encompassed by the present invention is a method of identifying an agent that modulates (enhances, inhibits) α_{1A}-AR (e.g., the biological activity, function and/or expression of α_{1A}-AR). The method comprises administering the agent to a transgenic mouse or a cell isolate whose genome comprises a recombinant nucleic acid sequence which comprises α_{1A}-adrenergic receptor (AR) and a marker peptide (e.g., an enhanced green fluorescent protein (EGFP)), wherein the α_{1A}-AR and the marker peptide are expressed as a fusion protein in the transgenic mouse. Whether α_{1A}-AR is modulated in the transgenic mouse or in the cell isolate is compared to a control mouse or cell, wherein if α_{1A}-AR is modulated in the transgenic mouse or cell isolate compared to the control mouse or cell, then the agent modulates α_{1A}-AR. Methods of determining whether α_{1A}-AR is modulated are provided herein, are known in the art, and include determining whether the agent modulates expression and/or one or more biological functions of α_{1A}-AR. Biological function of α_{1A}-AR include modulating neural stem cell differentiation; regulating differentiation or proliferation of neural stem cells or progenitor cells; enhancing expression of neural stem cells such as TAP cells, neuroblasts, oligodendrocyte progenitors; inhibiting production of astrocytes; enhancing cognitive function.

In a particular embodiment, the method includes determining whether the agent modulates (enhances, inhibits) the α_{1A}-AR activity of enhanced cognitive function such as in a transgenic non-human animal described herein. This method can further comprise determining whether the agent modulates (enhances, inhibits) the α_{1B}-AR activity of enhanced cognitive function. Methods for determining cognitive ability are provided herein (e.g., dry maze test, Morris water maze test) and are known to those of skill in the art.

The present invention is also directed to a method of identifying an agent that modulates (enhances, inhibits) neural stem cell or progenitor cell (e.g., TAP cell, neuroblast, oligodendrocyte progenitors) differentiation or proliferation by α_{1A}-AR. The method comprises administering the agent to a transgenic mouse or a cell isolate whose genome comprises a recombinant nucleic acid sequence which comprises α_{1A}-adrenergic receptor (AR) and a marker peptide (e.g., an enhanced green fluorescent protein (EGFP)), wherein the α_{1A}-AR and the marker peptide are expressed as a fusion protein in the transgenic mouse. Whether expression of one or more neural stem cell markers (e.g., α_{1A}-AR) is modulated in the transgenic mouse or in the cell isolate compared to a control mouse or cell is determined, wherein if the expression of the one or more neural stem cell marker is modulated in the transgenic mouse or cell isolate compared to the control mouse or cell, then the agent modulates neural stem cell or progenitor cell differentiation or proliferation by α_{1A}-AR.

The present invention is also directed to a method of determining whether a cell is a neural stem cell comprising identifying markers expressed on the cell, wherein if the markers comprise α_{1A}-AR, nestin, notch 1, vimentin and glia fibrillary acidic protein (GFAP), then the cell is a neural stem cell.

A method of regulating differentiation or proliferation of a neural stem cell or progenitor cell is also encompassed by the present invention. The method comprises contacting the neural stem cell or progenitor cell with an agent that modulates biological activity of α_{1A}-AR, expression of α_{1A}-AR or a combination thereof, in the neural stem cell or progenitor cell. In one embodiment, the differentiation or proliferation of the neural stem cell or progenitor cell is enhanced comprising contacting the neural stem cell or progenitor cell with an agent that enhances biological activity of α_{1A}-AR, expression of α_{1A}-AR or a combination thereof, in the neural stem cell or progenitor cell. In another embodiment, the neural stem cell differentiates into one or more cells selected from the group consisting of: a transiently amplifying progenitor (TAP) cell, a neuroblast, an oligodendrocyte and a combination thereof. In yet another embodiment, the differentiation of the neural stem cell or progenitor cell is inhibited comprising contacting the neural stem cell or progenitor cell with an agent that inhibits biological activity of α_{1A}-AR, expression of α_{1A}-AR or a combination thereof, in the neural stem cell or progenitor cell. In the methods of regulating differentiation or proliferation of a neural stem cell or progenitor cell, the methods can further comprise contacting the neural stem cell or progenitor cell with an agent that modulates biological activity of α_{1B}-AR, expression of α_{1B}-AR or a combination thereof, in the neural stem cell or progenitor cell.

The present invention is also directed to a method of treating a neurodegenerative disorder in an individual in need thereof, comprising administering to the individual an agent that regulates biological activity of α_{1A}-AR, expression of α_{1A}-AR or a combination thereof, in the individual. Examples of neurodegenerative disorders include Alzheimer's Disease, Parkinson's Disease, Multiple System Atrophy and spinal cord injuries.

Also encompassed by the present invention are methods of enhancing cognitive function (e.g., learning, memory) in an individual in need thereof comprising administering to the individual an agent that enhances biological activity of an α₁-AR, expression of an α₁-AR or a combination thereof, in the individual. The α₁-AR that is enhanced in the methods include α_{1A}-AR, α_{1B}-AR, β-AR and a combination thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C shows the results of experiments used to characterize the α_{1A}-AR-EGFP Transgenic Mice.

FIG. 1A is a schematic of transgenic constructs. A map of the transgene constructs showing the 4.4 kb fragment of the mouse α_{1A}-AR gene promoter driving the expression of a fusion protein of the human α_{1A}-AR-EGFP or the EGFP protein alone. The SV40 poly A tail sequence was also inserted for stability of the message.

FIG. 1B is a southern blot analysis. Using a probe as indicated in FIG. 1A, promoter only mice display a characteristic 0.8 kb band, while α_{1A}-AR-EGFP mice displayed a 2.8 kb band upon southern analysis.

FIG. 1C is a saturation binding graph. Bₘₐₓ determination (mean±SEM) was carried out via saturation binding in various α_{1A}-AR-positive and negative tissues using the non-selective a 1-antagonist [¹²⁵I] HEAT as the radioligand to measure total α₁-AR content. The asterisk (*) indicates significance (p<0.05) from the normal animals. N=4-5 mice per tissue performed in triplicate.

FIGS. 2A-2I show galactosidase expression in tissue samples of α_{1A}-AR knockout mice. α_{1A}-AR knockout mice in which the first exon of the α_{1A}-AR coding region was replaced by the LacZ gene, will express β-galactosidase activity (in blue) in cells expressed under the control of mouse α_{1A}-AR regulatory regions. β-galactosidase activity was determined as described in the methods. Regions expressing the α_{1A}-AR included cerebral cortex from the retrosplenial, motor, and somatosensory 1 (FIG. 2A); cerebral cortex from the somatosensory to entorhinal cortex (FIG. 2B); hippocampus (FIG. 2C); hypothalamic regions and the amygdale (FIG. 2D); midbrain region surrounding the aqueduct (FIG. 2E); hindbrain (FIG. 2F); cerebellum (FIG. 2G); spinal cord at the cervical level (FIG. 2H); spinal cord at the sacral level showing weak staining in the neuropil (FIG. 2I). Representative images repeated in 3 different mice. AG, anterior grey; AMYG, amygaloid; AWC, anterior white column; DG, dentate gyrus; DMH, dorsalmedial hypothalmus; DPO, dorsal periolivary region; DRI, dorsal raphe nucleus, inferior part; Ect, ectorhinal cortex; GC, gray commissure; I, intercalated nuclei amygdala; LEnt, lateral entorhinal cortex; LGC, lateral gray column; LH, lateral hypothalamic area; LSO, lateral superior olive; LWC, lateral white column; M1, primary motor cortex; M2, secondary motor cortex; Mo5, motor trigeminal nuclei; NS, nigrostriatal bundle; PAG, periaqueductal gray; PG, posterior grey; PnV, pontine reticular nucleus, ventrolateral; Pr5, principal sensory trigeminal nucleus; PRh, perirhinal cortex; RC, raphe cap; RSA, retrosplenial agranular cortex; RSG, retrosplenial granular cortex; S1Tr, somatosensory 1, barrel field; S2, secondary somatosensory cortex; SPO, superior paraolivary nucleus; VMH, ventromedial hypothalamus.

FIGS. 3A-3I show major areas in the brain that express the α_{1A}-AR. The α_{1A}-promoter mouse detects an even distribution in the cerebral cortex in the somatosensory 1, barrel field (S1 BF) (red arrows, FIG. 3A) but also contained a higher expressing cell type (white arrows, FIG. 3A). Similar region of the cerebral cortex in α_{1A}-AR mouse also shows an even distribution as the weaker expressing cells (red arrows, FIG. 3A) in the α_{1A}-promoter mouse (FIG. 3B). EGFP expression was prominent in the CA1-3 layers and the dentate gyrus of the hippocampus (FIG. 3C). Expression was most intense in the granular cells of the dentate gyrus (FIG. 3D) but cells could be visualized in the CA2-3 (FIG. 3E) and CA1 regions (FIG. 3F). In the midbrain, the α_{1A}-AR was prominently expressed in the periaqueductal grey, interpeduncular nuclei, deep mesencephalic nuclei, and in cells that lined the aqueduct (FIG. 3G). In the hindbrain, expression was seen in the pontine, superior olive, trigeminal nuclei, raphe pallidus, and in the dorsal raphe and raphe cap (FIG. 3H). In the cerebellum, α_{1A}-AR expression was expressed in all layers of the cerebellum, but stronger in the Purkinje cell layers and totally absent in white matter tracts (FIG. 3I). Blue color indicates nuclei. Mouse model used, either α_{1A}-AR-GFP tagged receptor (designated α_{1A}-AR) or promoter-EGFP alone (designated α_{1A}-promoter) is indicated in each image. Scale bars=20 μm in A, D; 40 μm in C, E-F; 100 μm in B, G-I. Representative images are shown from 3-4 different mice.

FIGS. 4A-4B show α_{1A}-AR expression in the spinal cord. (FIG. 4A) Cervical region. The α_{1A}-AR is expressed in anterior and lateral grey and white areas. (FIG. 4B) Sacral region. The α_{1A}-AR is expressed in both anterior and posterior grey areas, but no white columns. Mouse model used was the promoter-EGFP alone. Scale bars=100 μm. Representative images are shown from 3-4 different mice.

FIGS. 5A-5I show expression of the α_{1A}-AR with major cell type markers. EGFP was expressed in the cerebral cortex with cells that co-localized (in yellow) with the neuronal marker, NEUN (in red) (FIG. 5A). Some of the neurons expressing the α_{1A}-AR (in green) in the cerebral cortex also co-localized with the neurotransmitter GABA (in red) (FIG. 5B, arrows) or with NR1 (in red) (FIG. 5C, arrows), a subunit of the NMDA receptor. Cells in the dentate gyrus co-localized with GAD 65/67 (in red) (FIG. 5D) and the CA2-3 region of the hippocampus with NR1 (in red) (FIG. 5E). EGFP did not coexpress with α-synuclein (in red) (FIG. 5F) in the striatum. The α_{1A}-AR co-expressed with the NG2 marker for oligodendrocyte progenitor cells in the cerebral cortex (in red) (FIG. 5G, arrows) but was not expressed in cells containing markers for GFAP (in red) in the medulla (FIG. 5H), or CC1 (in red) in the raphe pallidus (FIG. 5I). Mouse model used was the promoter-EGFP alone (in green). Scale bars=10 μm in F, H; 20 μm in B-C, E, G; 50 μm in D; 100 μm in FIGS. 5A, 5I. Representative images are shown from 3-4 different mice.

FIGS. 6A-6D show the electrophysiological response of an α_{1A}-AR-EGFP-expressing interneuron to α-AR stimulation. (FIG. 6A) Diagram of the rat hippocampal slice preparation showing the major pathways, layers and cell types of the hippocampus. Details of hippocampal CA1 region are shown illustrating the placement of the recording electrode on an interneuron in the stratum oriens. A subpopulation of interneurons in this area is known to form inhibitory GABAergic synapses with the CA1 pyramidal cells and to respond to α₁-AR stimulation. (FIG. 6B) Images of an α_{1A}-AR-EGFP positive hippocampal CA1 interneuron located in stratum oriens as depicted in A. Top: Fluorescent image of this EGFP interneuron. Bottom. Infrared image of this same EGFP positive interneuron. Note the recording electrode attached to this cell. (FIG. 6C) Consecutive current traces of the cell-attached patch recording from the hippocampal interneuron shown in B illustrating spontaneous action potentials (APs) recorded during the pharmacological manipulations shown in (FIG. 6D) Frequency versus time plot of the APs recorded from the interneuron shown in (FIG. 6B). Application of 6FNE (10 μM) caused a large increase in AP frequency over that recorded in control conditions which was fully reversible with wash. Inset: Effect of α-AR stimulation (6FNE) on action potential generation in α_{1A}-AR-EGFP positive hippocampal CA1 interneurons (n=4). Mouse model used was the α_{1A}-AR-GFP tagged receptor. Statistical significance of the difference in AP frequency between control and 6FNE is indicated by an asterisk (*, P<0.05).

FIG. 7 is a model for neural stem cell (NSC) differentiation and α₁-AR involvement.

FIG. 8 is a schematic of the Notch1 signaling pathway.

FIG. 9 shows α₁-AR expressing cells are found in the subventricular zone (SVZ) and the rostral migratory stream (RMS) and have a migratory phenotype.

FIG. 10A shows the α-AR in promoter only mice is abundant in the SVZ, where Nestin is located.

FIG. 10B shows X-gal stains in a similar pattern in the α₁-AR knockout (KO) mice in the SVZ.

FIGS. 11A-11B shows that the α₁-AR promoter only mice express EGFP in the cytoplasm and that the promoter only cells also express Vimentin in the SVZ (FIG. 11A) or Nestin (FIG. 11B) near the ependymal layer. Scale bar=10 μM.

FIGS. 12A-12C show α₁-AR expressing cells are expressed in the SVZ along with Notch1; FIG. 12A is a magnified image from the boxed area in FIG. 12B. FIG. 12C shows that in the RMS, α₁-AR cells appear to be surrounded by Notch1 cells and are likely neuroblasts. Scale bar=10 μM; 2M old.

FIG. 13 shows some α₁-AR promoter only cells express D1x2 in the nucleus in the SVZ and the RMS; however, an α₁-AR green cell type exists that does not express D1x2. Scale bar=10 μM; Mice are 2 months old.

FIGS. 14A-14C show a normal embryonic mouse neurosphere (FIG. 14A); normal embryonic neurospheres differentiated for 7 days show neurons, astrocytes and oligodendrocyte progenitors (FIG. 14B); and a proliferating neurosphere shows background (FIG. 14C).

FIGS. 15A-15C show normal nonclonal embryonic mouse neurospheres placed in proliferation media (+EGF/FGF+)+phenylephrine for 3, 7 or 10 days differentiate into neurons, astrocytes and oligodendrocyte progenitors; note reduction in GFAP expression with prolonged phenylephrine exposure and bipolar nature of MAP2/NG2 development. Images were taken at the same gain value on all three lasers during confocal analysis. Scale bar=10 μM.

FIG. 16 is a graph showing phenylephrine (Phe) enhances the differentiation of normal embryonic mouse neurospheres grown in differentiation media (−EGF/FGF); this effect can be blocked with prazosin (Prz), an α₁-AR antagonist, but not with propranolol (prop) a β-AR antagonist.

FIG. 17 is a western blot of immunoprecipitated Notch1 and Nestin proteins in normal, α₁-AR GFP, and α₁-AR KO mouse models. α₁-AR KO models express lower levels of the transmembrane-containing subunit of Notch1 (arrow 1) and the cleaved NICD signaling product of Notch1 (arrow 2). However, α₁-AR KO mice express higher levels of Nestin (arrow 3). This indicates that α₁-ARs can modulate the protein levels of Notch1 and nestin, crucial proteins in neurogenesis. Samples are SVZ isolated regions from adult mouse brain (2M) and both western blots are from the same samples.

FIG. 18A is a bar graph showing that neonatal neurospheres isolated from CAM α_{1A}-AR mice have a lower ability to regenerate neurospheres than normal or α_{1A}-AR-KO mice, demonstrating that the α_{1A}-AR can modulate neural stem cell function.

FIG. 18B is a proliferation curve of neurospheres from embryonic normal (green), normal neonatal (purple) or CAM α_{1A}-AR mice (orange), CAM α_{1B}-AR (black), α_{1A}-KO (red), and α_{1B}-KO (blue), indicating that both the α_{1A} and α_{1B} can decrease the proliferation of neurospheres consistent with their ability to enhance differentiation.

FIG. 19A is a saturation binding curve showing that normal neonatal neurospheres express 140 fmoles/mg protein of both a i-AR subtypes, demonstrating that the receptor protein is indeed expressed in neurospheres.

FIG. 19B is a competition binding curve with 5-methylurapidil showing that normal neonatal neurospheres express 40% of the α_{1A}-AR subtypes and 60% of the α_{1B}-AR subtypes.

FIG. 20A shows that normal neonatal neurospheres differentiate into all three cell types upon incubation with serum (2% fetal bovine serum (FBS)), demonstrating that these neurospheres are pluripotent and contain neural stem cells.

FIG. 20B shows that CAM α_{1A}-AR neonatal neurospheres cultured with serum (2% FBS), differentiated into all three cell types but were mostly neurons (magenta, MAP2) and NG2 oligodendrocytes (green, NG2), and had very little astrocytes (red, GFAP), consistent with enhanced differentiation ability of these neurospheres.

FIG. 20C shows that serum-cultured α_{1A}-knock out (KO) neonatal neurospheres regained the ability to differentiate into astrocytes but had reduced levels of neurons and NG2 cells than normal cells, consistent with an opposite result from the CAM α_{1A}-AR.

FIGS. 21A-21C show normal neonatal neurospheres placed in B27 media (+epidermal growth factor/fibroblast growth facto (+EGF/FGF)) and phenylephrine (Phe) for 0 (FIG. 21A, control), 3 (FIG. 21B) or 10 (FIG. 21C) days differentiates into neurons (MAP2 in magenta), astrocytes (GFAP in red) and oligodendrocyte precursors (NG2 in green). Nuclei are blue (DAPI). All three cell types are expressed after 3 days of stimulation but prolong stimulation after 10 days reduced or eliminated GFAP expression. This figure differs from FIGS. 15A-15C only in the use of a different fluorescent label that give clearer images.

FIGS. 22A-22B show neonatal CAM α_{1A}-AR basal (FIG. 22A) and day 10 α_{1A}-AR stimulation (Phe) (FIG. 22B). Both conditions produced only neurons and NG2 oligodendrocyte precursors, no astrocytes, since the receptor is already activated, there is no further enhancement from Phe.

FIGS. 23A-23B show knockout (KO) of the α_{1A}-AR in neonatal neurospheres restores basal expression of astrocytes (FIG. 23A, red). Astrocytes are still expressed after 10 days of phenylephrine (phe) stimulation, opposite to neurospheres isolated from the CAM α_{1A}-AR or normal mice (FIG. 23B).

FIGS. 24A-24B show graphs of real time polymerase chain reaction (PCR) of RNA isolated from neonatal neurospheres derived from normal (black squares), CAM α_{1A}-AR (blue triangles) and α_{1A}-KO (red circles) mice after differentiation with 2% serum for 0, 1, 3 and 7 days, demonstrating that α_{1A}-AR can modulate the expression of key genes known to be involved in neurogenesis.

FIG. 25 is a model of neuronal differentiation showing factors believed to be involved in neuronal differentiation, represented at their respective stage in the neurogenic process. Many of these genes were tested for their ability to be modulated by the α_{1A}-AR in FIG. 24.

FIGS. 26A-26B show graphs of real time PCR showing that phenylephrine stimulation induced increased mRNA expression in normal neonatal neurospheres (black squares) of all of the above genes involved in neurogenesis and neuronal differentiation. α_{1A}-KO (red circles) and CAM α_{1A}-AR (Blue triangles) reduced expression except for nestin. CAM a IA has reduced expression because these cells are already differentiated and could no longer be modulated.

FIG. 27 is a bar graph showing α_{1A}-AR induced Ngn-2 gene expression in normal neonatal neurospheres can be blocked with the MEK inhibitor (PD), the PI3K inhibitor (LY) or with the PKC inhibitor (GO) but not with the p38 inhibitor (SB), indicating that α_{1A}-AR modulates neurogenic gene expression through pathways commonly associated with α_{1A}-AR signaling.

FIGS. 28A-28F show astrocytic labeling in vivo (GFAP in red) of normal mouse neurogenic regions (FIG. 28A) or from the CAM α_{1A}-AR mice (FIG. 28B). GFAP labeling in the normal mouse hippocampus (FIG. 28C), CAM α_{1A}-AR hippocampus (FIG. 28D), normal SVZ (FIG. 28E) and CAM α_{1A}-AR SVZ (FIG. 28F) are shown. CAM α_{1A}-AR mice display less GFAP labeling in neurogenic regions, consistent with in vitro neurosphere data that α_{1A}-AR inhibits their development. Mice are less than 1 month old. Nuclei are blue.

FIGS. 29A-29F show in vivo astrocytic (GFAP, red), neurons (magenta, MAP2) and NG2 oligodendrocytes (green) in normal adult mice in the hippocampus (FIG. 29a), SVZ (FIG. 29B), in CAM α_{1A}-AR in the hippocampus (FIG. 29C) or SVZ (FIG. 29D) or in the α_{1A}-KO mice in the hippocampus (FIG. 29E) or SVZ (FIG. 29F). CAM a IA mice have more neurons and NG2 oligodendrocytes than normal or α_{1A}-KO, consistent with in vitro neurosphere data.

FIGS. 30A-30E are graphs of the results of cognitive testing on adult mice. FIG. 30A is a graph showing the results of cognitive dry maze testing on adult mice showing that CAM α_{1A} and CAM α_{1B}-AR mice have enhanced learning behavior. FIG. 30B is a graph showing the results of the dry maze memory test for normal (black), α_{1A}-KO (magenta) and CAM α_{1A}-AR (green). CAM α_{1A}-AR has better memory performance than normal or KO mice. FIG. 30C is a graph showing the results of the dry maze memory test for normal (black), α_{1B}-KO (blue) and CAM α_{1B}-AR (red). CAM α_{1B} mice quickly lost the ability to remember the maze. FIG. 30D is a graph showing the results of the learning part of the Morris swim test for all mice. Both the CAM a IA and CAM α_{1B} mice were involved in learning the swim test. FIG. 30E is a graph showing the results of the reversal of the platform in the Morris swim test for all mice. Here, CAM α_{1B} mice were better at remembering the swim test. While results can be variable in mice, data indicate that both the α_{1A} and α_{1B} are involved in enhancing cognitive functions, with both subtypes enhancing learning but the α_{1A} is likely better at memory.

FIG. 31 is a graph of a competition ligand binding curve with ICI, 118551 (a β₂-AR selective agonist) and the radiolabeled ¹²⁵ICYP which labels β-AR receptors, a related receptor to the α₁-AR, showing that normal neonatal neurospheres express 86% of the β₁-AR subtypes and 14% of β₂-AR subtype, indicating that β-ARs are also involved in regulating neurogenesis.

### DETAILED DESCRIPTION OF THE INVENTION

α₁-Adrenergic receptors (ARs) are not well defined in the central nervous system. The particular cell types and areas that express these receptors are uncertain because of the lack of high avidity antibodies and selective ligands. Described herein are transgenic mice that either systemically over-express the human α_{1A}-AR subtype fused with the enhanced green fluorescent protein (EGFP) or expresses the EGFP protein alone under the control of the mouse α_{1A}-AR promoter. The transgenic model described herein is confirmed against an α_{1A}-AR knockout mouse, which expresses the LacZ gene in place of the coding region for the α_{1A}-AR. Using these models, cellular localization of the α_{1A}-AR in the brain, at the protein level, has now been determined. The α_{1A}-AR or the EGFP protein is expressed prominently in neuronal cells in the cerebral cortex, hippocampus, hypothalamus, midbrain, pontine olivary nuclei, trigeminal nuclei, cerebellum and spinal cord. The types of neurons were diverse and the α_{1A}-AR co-localized with markers for glutamic acid decarboxylase (GAD), gamma-aminobutyric acid (GABA), and N-methyl-D-aspartate (NMDA) receptors. Recordings from α_{1A}-AR EGFP-expressing cells in the stratum oriens of the hippocampal CA1 region confirmed that these cells were interneurons. Expression of the α_{1A}-AR in mature astrocytes, oligodendrocytes, or cerebral blood vessels were not detected, however, the α_{1A}-AR was detected in oligodendrocyte progenitors. α_{1A}-AR is abundant in the brain, expressed in various types of neurons, and likely regulates the function of oligodendrocyte progenitors, interneurons, and GABA/NMDA containing neurons.

Described herein are model systems that examine the properties of the α₁-AR proteins by targeting systemic overexpression of a receptor fused to EGFP in tissues that should contain the endogenous receptors. An α_{1A}-AR promoter-only EGFP construct (without the α_{1A}-AR) is described also. Detection of the systemic distribution of α_{1B}-AR expression is achieved by using a large fragment of the mouse α_{1B}-AR promoter (Zuscik et al., Mol. Pharmacol., 56:1288-1297 (1999); Papay et al., J. Comp. Neurol., 478:1-10 (2004)) and the promoter's in vivo specificity has been previously verified by in situ mRNA comparison (Zuscik et al., Nat. Med, 6:1388-1394 (2000)). The location of the α_{1B}-AR in the mouse brain has been previously characterized (Papay et al., J. Comp. Neurol., 478:1-10 (2004)). Described herein is the brain localization of the α_{1A}-AR. While mRNA localization of the α_{1A} and α_{1B}-ARs are quite distinct (McCune et al., Neurosci., 57:143-151 (1993); Pieribone et al., J. Neurosci., 14.4252-4268 (1994); Nicholas et al., Trends Pharmacol. Sc., 17:245-255 (1996); Domyancic and Morilak, J. Comp. Neurol., 474:108-122 (1997)), at the protein level both α_{1A}- and α_{1B}-ARs are expressed in similar regions in the brain but with differences in abundance. As shown herein, the α_{1A}-AR subtype modulates adult neurogenesis in the mouse brain.

Adrenergic receptors (ARs) are glycosylated integral membrane proteins that are activated by selectively binding the catecholamines, norepinephrine and epinephrine (Graham R M and Lanier S M, In: The Heart and Cardiovascular System (ed. HA Fozzard et al.), pp 1059-1095 (1989)). ARs, as determined by their different pharmacological specificities, physiology, and primary structure, are classified as α₁, α₂, β₁, β₂, and β₃. By transducing the external chemical stimulus into an intracellular signal, these receptors regulate the sympathetic nervous system, and thus, play a crucial role in a variety of tissue specific responses. ARs belong to the superfamily of at least 1000 distinct G-protein-coupled receptors sharing a common structural motif (Strader C D., et al., FASEB J, 3: 1825-1832 (1989)). This motif consists of seven transmembrane (TM) domains of 20 to 28 hydrophobic amino acids, which interestingly, form the ligand-binding site in an analogous manner to the visual transducing protein rhodopsin, where the ligand 11-cis retinal, sits in the binding pocket formed within the membrane bilayer (Oprian D., et al., FENS, 3:20-28 (1991)).

α₁-ARs are a group of heterogeneous but related proteins. The cDNAs are separate gene products and have been isolated for three subtypes (α_{1A}, α_{1B}, α_{1D}), all three of which we have cloned, characterized and remain a major contributor to the study of their pharmacology, structure-function and physiological effects (Perez, D M., et al., Mol. Pharmacol. 40:876-883 (1991); Ramaro, C S., et al., J. Biol. Chem., 267:21936-21945 (1992); Perez, D M., et al., Mol. Pharmacol., 46:823-883; Ross, S A., et al., Cardiovascular Res., 60:598-607 (2003); Gonzalez-Cabrera, P J., et al., Endocrinilogy, 145:5157-5167 (2004); Rorabaugh, B R., et al., Cardiovascular Res., 65:436-445 (2005)). α₁-ARs are delineated according to their primary sequence and their affinity for subtype-selective antagonists, such as niguldipine and 5-methylurapidil (Hanft, G., et al., J. Pharm. Pharmacol., 41:714-716 (1989)). α_{1A}-ARs have a high affinity for these competitive antagonists. Conversely, α_{1B}- and α_{1D}-ARs have a low affinity for these same ligands. Examples of α₁-AR-selective antagonists, which block all of the α₁-AR subtypes with equal ability, are prazosin and HEAT. Phenylephrine is an α₁-AR selective agonist.

Studies of α₁-AR signaling pathways indicate a heterologous system, especially dependent upon the cell type studied (Perez, D M., et al., Mol. Pharmacol., 44:784-795 (1993)). In most cases, however, α₁-AR stimulation leads to activation of phospholipase C, which hydrolyzes phosphotidylinositol-4, 5-bisphosphate, yielding inositol-1,4, 5-trisphosphate and diacylglycerol (Berridge, M J., et al., Nature, 361:315-325 (1993)), second messengers that promote intracellular calcium mobilization and protein kinase C activation, respectively. This pathway involves receptor coupling to G_{q} (Cotecchia, S., et al., J. Biol. Chem., 265:63-69 (1990)).

The α₁-ARs are not well understood of the central adrenergic receptors. In the somatosensory areas of the cortex, α₁-AR activation has been found to increase the excitation seen after administration of glutamate or acetylcholine (Mouradian, R D, et al., Brain Res., 546:83-95 (1991)). α₁-ARs also cause excitatory responses in subcortical areas such as the reticular thalamic nucleus, dorsal raphe and spinal motor neurons. α₁-ARs may modulate both weak and strong activation of the pyramidal neurons in the neocortex and may play a role in long-term potentiation. α₁-ARs may affect many brain functions via non-neuronal mechanisms since they may be localized to glial cells (Lerea, L S., et al., Glia, 2:135-147 (1989)).

From the recent α₁-AR knockout (KO) models, most of the characterization has been in cardiovascular areas. Neuroscience information from the KO models that has been published is that the α_{1B}-AR is thought to be involved in the interactions between noradrenergic and dopaminergic neurons, specifically in the nucleus accumbens, which controls the actions of psychostimulants, such as D-Amphetamine. The α_{1B}-AR was shown to control the locomotor and rewarding effects of opiates and amphetamine by controlling the release, by amphetamine, of extracellular dopamine (Drouin, C., et al., J. Neurosci., 22:2873-2884 (2002)). The α_{1D}-AR is poorly expressed throughout the brain as assessed by binding studies and from the α_{1D}-AR KO mouse (Tanoue, A., et al., J. Clin. Invest., 109:765-775 (2002)). Therefore, its role in neurotransmission is likely to be minor. The α_{1A}-AR KO mice were noticed to have seizures (Rokosh, D G., et al., PNAS, 99:9474-9479 (2002)).

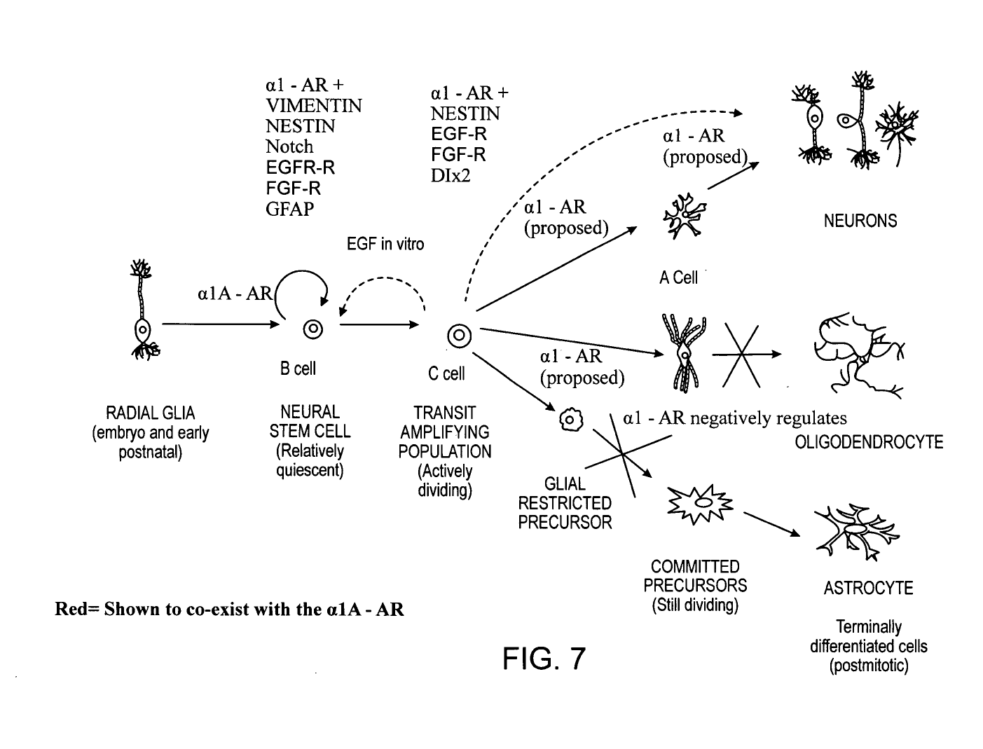

In the adult mammalian brain, the genesis of new neurons has been consistently documented in the SVZ of the lateral ventricles. From the SVZ, newly generated neurons reach their final destination in the olfactory bulb after migration through a well-defined path called the rostral migratory stream (RMS) (Johansson, C B., et al., Cell, 96(1):25-34 (1999)). Four main cell types are found in the SVZ, migrating neuroblasts, neural stem cells, ependymal cells, and transitory amplifying progenitor (TAP) cells. Ependymal cells form a monolayer that lines the ventricle. NSCs function as the primary precursor of rapidly dividing transit-amplifying (TAP) cells that generate the restricted precursors (FIG. 7). The NSCs (Type B cell) surrounds the neuroblast chains (Type A cell) and TAP cells (Type C cell). Type C cells generate the progenitors and the migrating chain of neuroblasts. Notch, GFAP and vimentin are expressed in NSCs. Not all astrocytes are neural stem cells, so it becomes important to distinguish them from the stem cell population. Nestin is a protein expressed by neural stem cells (Lendahl, U., et al., Cell, 60(4):585-595 (1990)) but also TAP cells and is highly expressed in ependymal cells and at lower levels in the rapidly proliferating subventricular zone progenitor cells in the adult (Doetsch, F., et al., J. Neurosci., 17(13):5046-5061 (1997)). The TAP cell is the most actively dividing of the SVZ cell types. D1x2 is a homeobox-containing transcription factor found in TAP and migrating neuroblasts (Doetsch, F., et al., Neuron, 36(6):1021-1034 (2002)).

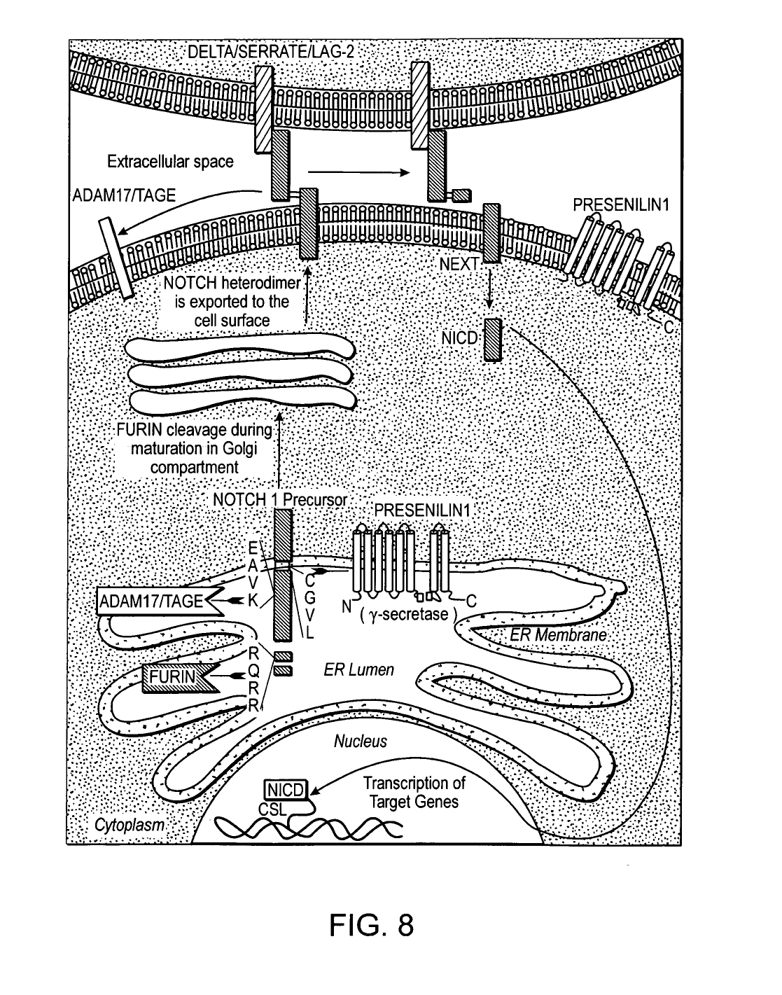

Notch1 is a single-pass transmembrane receptor that normally controls cellular differentiation in hematopoetic cells. Although synthesized as a 350 kD precursor glycoprotein, processing by a furin-like protease cleaves Notch 1 into two non-covalently associated subunits, an extracellular ECN subunit and a transmembrane (NEXT) subunit (FIG. 8). ECN contains 36 N-terminal EGF-like modules. Within the EGF repeat region lie binding sites for activating ligands, such as delta, serrate, and jagged.

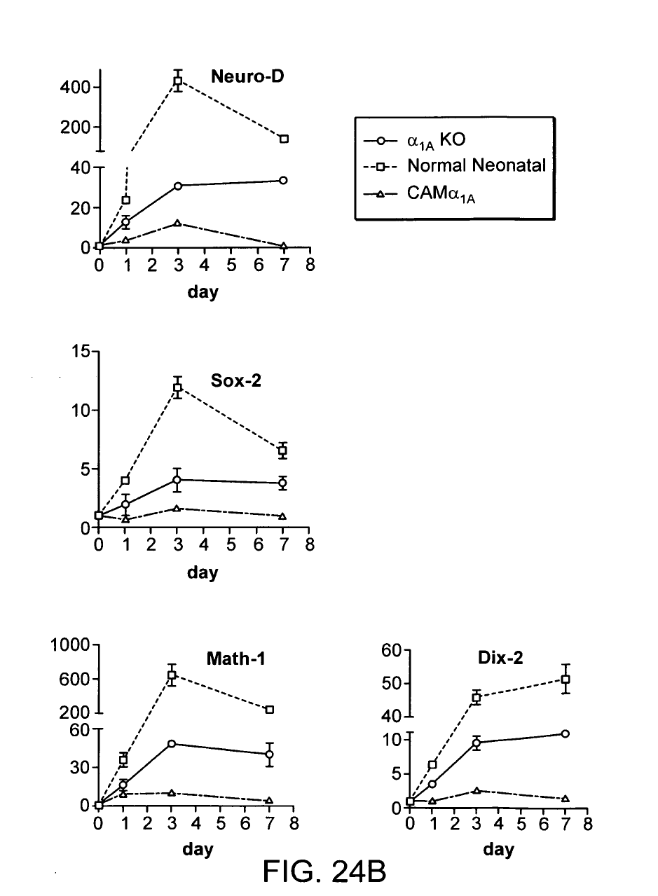

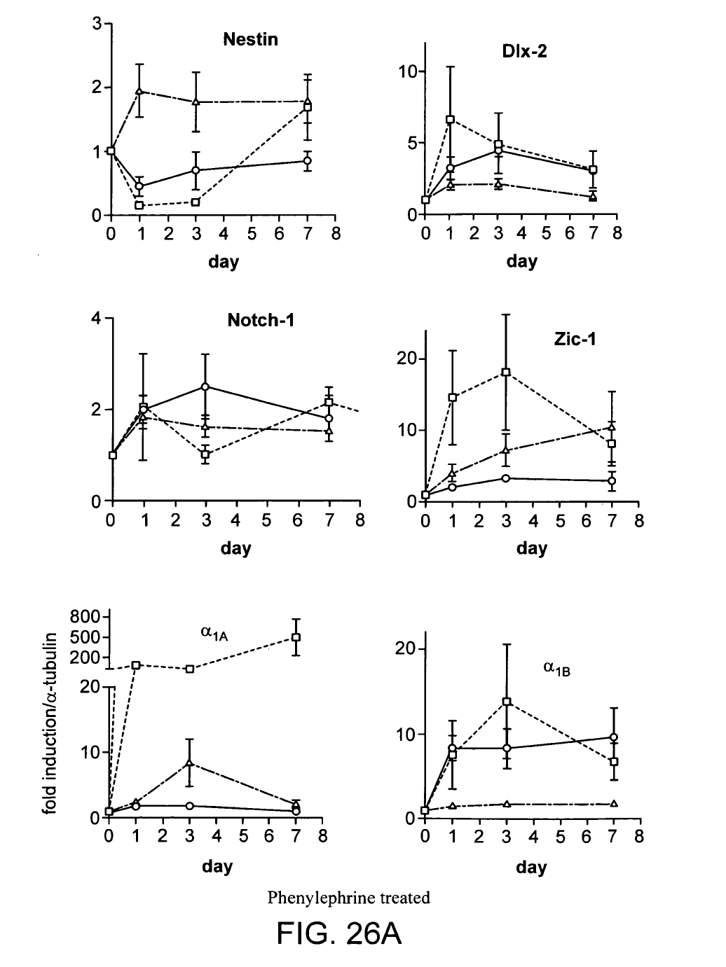

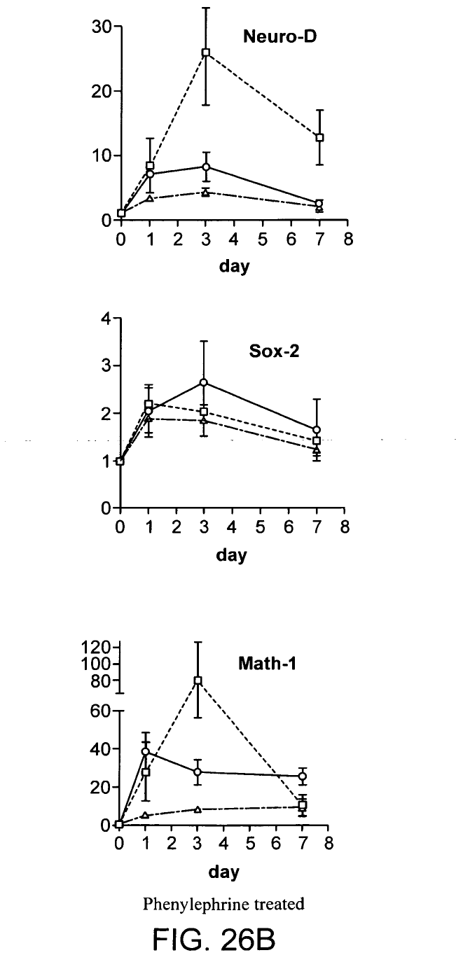

The current working model for signaling by Notch and related receptors is that ligand binding triggers a cascade of proteolytic cleavages that release the intracellular portion of Notch from the membrane (Mumm, J S., et al., Mol. Cell, 5:197-206 (2000); Logeat, F., et al., Proc. Natl. Acad. Sci. USA, 95(14):8108-8112 (1998)). The untethered intracellular fragment of Notch (NICD) then migrates to the nucleus where it participates in the activation of transcription of Notch-responsive genes. Evidence is provided herein that α₁-AR signaling may modify the cleavage of Notch1. As shown in FIG. 17, FIG. 24 and FIG. 26, it is likely that α₁-ARs modulate the protein and RNA levels of Notch 1 and Nestin, crucial proteins in neurogenesis.

Glycoprotein 130 and its ligands leukemia inhibitory factor (LIF), ciliary neurotrophic factor (CNTF), interleukin 6 (IL6) regulate Notch 1 expression and maintains self-renewal of NSCs (Chojnacki, A., et al., J. Neurosci., 23(5):1730-1741 (2003)). α₁-ARs regulate the secretion of IL-6 and LIF and act through gp130 to differentially regulate Jak/Stat3 signaling (30). α₁-ARs also potentially can secret VEGF (Gonzalez-Cabrera, P J, et al., Mol. Pharmacol., 63:1104-1116 (2003)), suggested to increase proliferation of neuronal precursors (Jin, K., et al., Proc. Natl. Acad. Sci., USA, 99(18):11946-11950 (2002)). The choroid plexuses of all ventricles receive a well-developed adrenergic and cholinergic innervation reaching both the secretory epithelium and the vascular smooth muscle cells. The choroid plexus appears to be source of hormones and growth factors, and is a site of VEGF production (Schanzer, A., et al., Brain Pathol., 14(3):237-248 (2004)). The choroid plexus epithelium could be a site of catecholamine secretion and activation as was found for serotonin and its receptor (Esterle, T M, et al., J. Neurosci., 12(12):4775-4782 (1992)). The α₁-ARs have also been suggested to transactivate EGF-R (Zhang, H., et al., Circ. Res., 95(10):989-997 (2004); Grau, M., et al., Endocrinology, 138(6):2601-2609 (1997)) and secrete FGF (Shi, T., et al., Mol. Pharm., 70:129 (2006)), factors needed to maintain NSC nitches.

A mouse model that systemically express the α_{1A}-AR subtype using the endogenous promoters for the mouse receptors is described herein. Endogenous expression has been confirmed in the α_{1A}-AR-EGFP mice using the α_{1A}-AR knockout (KO) mouse, in which the first exon of the α_{1A}-AR was replaced by the LacZ gene. Therefore, β-galactosidase expression is under the control of the endogenous promoter for the mouse α_{1A}-AR. As shown herein, β-galactosidase expression in the α_{1A}-AR KO correlates to the EGFP signal in the α_{1A}-AR-EGFP transgenic brain, confirming promoter fidelity and that the α_{1A}-ARs are contained in cells in the neurogenic regions of the brain in vivo.

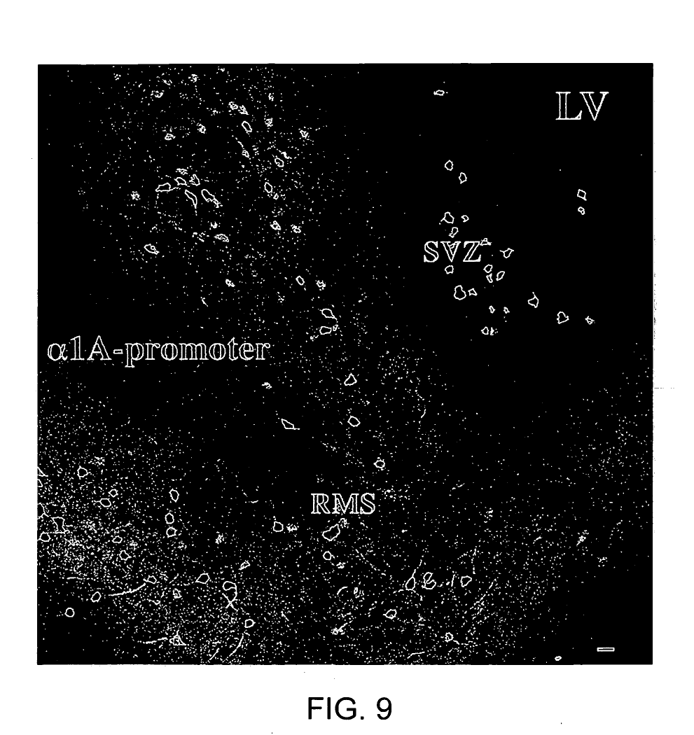

Models that overexpress the α₁-ARs with constitutively active mutations (CAMs) that cause the receptor to be activated without agonists have been described. These models are useful because of the lack of highly selective agonists that stimulate the receptors. These mutations have been previously characterized both in vitro and in vivo to verify the constitutive activity (Zuscik, M J., et al., Nature Med., 6:1388-1394 (2000); Hwa, J., et al., Biochem., 36:633-639 (1997); Hwa, J., et al., J. Biol. Chem., 271:7956-7964 (1996); Perez, D M., et al., Mol. Pharmacol., 49:112-133 (1996); Kjelsberg, M A, et al., J. Biol. Chem., 267:1430-1433 (1992); Rorabaugh, B R, et al., Cardiovascular Res., 65:436-445 (2005)). Described herein are α₁-AR subtype models with EGFP tags. Antibodies against these receptors have been categorically rejected in endogenous tissues due to poor avidity. With EGFP tagged receptors expressed in an endogenous way, these receptors can be detected using the EGFP protein. An α_{1A}-AR promoter only-EGFP construct (i.e. without the receptor) that expresses the EGFP protein in the same tissues as the α_{1A}-AR is also described herein. No differences in the localization of the α_{1A}-AR receptor with either construct (receptor-tagged or promoter only) were found. In FIG. 9, α_{1A}-AR expressing cells in the SVZ and RMS with cells further down the RMS developing neuronal-like processes, are shown. This indicates that the α_{1A}-AR is involved throughout the neurogenic process.

Accordingly, provided herein is a transgenic non-human mammal whose genome comprises a recombinant nucleic acid sequence comprising all or a functional portion of an α_{1A}-adrenergic receptor (AR) and a marker peptide operably linked to all or a functional portion of an α_{1A}-AR promoter, wherein the α_{1A}-AR and the marker peptide are expressed as a fusion protein in the transgenic non-human mammal. In a particular embodiment, the fusion protein is overexpressed in the transgenic non-human mammal. As used herein “overexpressed” and “overexpression” refer to increased expression of the α_{1A}-AR, which is fused to the marker peptide, compared to expression of the α_{1A}-AR in a wild type non-human mammal. In a particular embodiment, the wild type non-human mammal is of the same species as the transgenic non-human mammal. The amount of overexpression is dependent upon the tissue examined and the promoter used. One of skill in the art will appreciate that selection of parameters such as the targeting construct and the promoter will provide the desired amount overexpression, however, to be physiologically relevant, the amount of overexpression is generally about a 2-fold (e.g., in the heart), about a 3-fold, about a 4-fold, about a 5-fold (e.g., in the kidney, brain) or about a 6-fold increase in expression compared to a wild type mammal.

The present invention also provides methods of producing a transgenic non-human mammal whose genome comprises a recombinant nucleic acid sequence comprising an α_{1A}-AR and a marker peptide, as well as targeting constructs for use in such methods. The invention also provides a source of cells (for example, tissue, cells, cellular extracts, organelles) and animals useful for elucidating the function of α_{1A}-AR in intact animals. Further aspects of the invention provide methods for the identification of agents that modulate neural stem cell or progenitor differentiation of cells comprising α_{1A}-AR in vivo or in vitro; methods of determining whether a cell is a neural stem cell; methods of regulating differentiation or proliferation of a neural stem cell or progenitor cell; and methods of treating neurodegenerative diseases or conditions (e.g., cognitive impairment).

In one embodiment, the invention is directed to a transgenic mouse whose genome comprises a recombinant nucleic acid sequence which comprises a mouse α_{1A}-adrenergic receptor (AR) promoter operably linked to a human α_{1A}-AR and an enhanced green fluorescent protein (EGFP), wherein the human α_{1A}-AR and the EGFP are expressed as a fusion protein in the transgenic mouse and the EGFP is fused to the C-terminus of the human α_{1A}-AR. In a particular embodiment, the α_{1A}-AR-EGFP fusion protein is overexpressed in the transgenic non-human mammal.

In yet another embodiment, the genome of the transgenic non-human mammal comprises a recombinant nucleic acid sequence comprising a marker protein, under the control of all or a functional portion of an α_{1A}-AR promoter.

Any suitable non-human mammal can be used to produce the transgenic non-human mammal described herein. For example, a suitable mammal can be, a rodent (e.g., mouse, rat), a rabbit, a pig, a sheep, a goat or a cow.

The α_{1A}-AR expressed in the transgenic non-human mammal can be derived from any suitable vertebrate source such as a primate (e.g., human, chimpanzee), a rodent (e.g., mouse, rat), a rabbit, a pig, a sheep, a goat or a cow. In addition, as described herein all or a functional portion of the α_{1A}-AR is expressed in the transgenic non-human mammal. As used herein a “functional portion of α_{1A}-AR” refers to a portion of α_{1A}-AR that is large enough to retain its biological activity (e.g., modulating neural stem cell differentiation; regulating differentiation or proliferation of neural stem cells or progenitors; expression on neural stem cells; inhibiting production of astrocytes).

Any suitable marker peptide that can be detected (directly or indirectly) in the transgenic non-human mammal of the present invention can also be used in the methods of the present invention. In a particular embodiment, the marker peptide is a fluorescent peptide such as green fluorescent peptide (GFP) and/or enhanced green fluorescent peptide (EGFP). In another embodiment, the marker peptide can be a peptide that is recognized by a highly avid antibody such as c-myc, Flag or other commercially available protein tag.

As described herein, α_{1A}-adrenergic receptor (AR) and a marker peptide are operably linked to all or a functional portion of an α_{1A}-AR promoter, wherein the α_{1A}-AR and the marker peptide are expressed as a fusion protein in the transgenic non-human mammal. As used herein, a “functional portion of an α_{1A}-AR promoter” is a fragment of an α_{1A}-AR promoter that is large enough to retain its biological activity of directing expression of a peptide to which it is operably linked (large enough to direct expression of the α_{1A}-AR promoter-marker peptide fusion protein when the portion of the α_{1A}-AR promoter is operably linked to the fusion protein). Examples of such functional portions are provided in the exemplification and in the Zuscik et al., Mol. Pharmacol., 56:1288-1297 (1999) and Papay et al., J. Comp. Neurol., 478:1-10 (2004) references.

The present invention is also directed to methods of producing the transgenic non-human mammals described herein. In one embodiment, the invention is directed to a method of producing a transgenic non-human mammal whose genome comprises a recombinant nucleic acid sequence comprising an α_{1A}-AR and a marker peptide, wherein the α_{1A}-AR and the marker peptide are expressed as a fusion protein in the transgenic non-human mammal. Briefly, the standard methodology for producing a transgenic embryo requires introducing a targeting construct, which is designed to integrate by homologous recombination with the endogenous nucleic acid sequence of the targeted gene, into a suitable ES cells. The ES cells are then cultured under conditions effective for homologous recombination between the recombinant nucleic acid sequence of the targeting construct and the genomic nucleic acid sequence of the host cell chromosome. Genetically engineered stem cells that have a genotype comprising an α_{1A}-AR fused (in frame) to a marker peptide are identified and introduced into an animal, or ancestor thereof, at an embryonic stage using standard techniques which are well known in the art (for example, by microinjecting the genetically engineered ES cell into a blastocyst). The resulting chimeric blastocyst is then placed within the uterus of a pseudo-pregnant foster mother for the development into viable pups. The resulting viable pups include potentially chimeric founder animals whose somatic and germline tissue comprise a mixture of cells derived from the genetically-engineered ES cells and the recipient blastocyst. The contribution of the genetically altered stem cell to the germline of the resulting chimeric mice allows the altered ES cell genome which comprises the fusion protein to be transmitted to the progeny of these founder animals thereby facilitating the production of transgenic non-human mammals whose genome comprises a recombinant nucleic acid sequence comprising an α_{1A}-AR and a marker peptide operably linked to all or a functional portion of an α_{1A}-AR promoter.

In another embodiment, the present invention is directed to a method of producing a transgenic non-human mammal whose genome comprises a recombinant nucleic acid sequence comprising an α_{1A}-AR promoter and a marker peptide, wherein the marker peptide is expressed under the control of the α_{1A}-AR promoter (operably linked) in the transgenic non-human mammal. The method comprises introducing a targeting construct which comprises the recombinant nucleic acid sequence comprising the α_{1A}-AR promoter and the marker peptide, into a pronuclei of an embryo. The embryo can then be introduced into a pseudo-pregnant non-human female mammal under conditions in which the non-human female mammal gives birth to a chimeric transgenic non-human mammal whose genome comprises the recombinant nucleic acid sequence comprising the α_{1A}-AR promoter and the marker peptide. The chimeric transgenic non-human mammal is bred with a second mammal to generate heterozygous F1 progeny that are heterozygous for the recombinant nucleic acid sequence comprising the α_{1A}-AR promoter and the marker peptide; and the heterozygous F1 progeny are crossbred under conditions in which a transgenic non-human mammal whose genome comprises a recombinant nucleic acid sequence comprising an α_{1A}-adrenergic receptor (AR) promoter and a marker peptide, wherein the marker peptide is expressed under the control of the α_{1A}-AR promoter in the transgenic non-human mammal, and homozygote F2 progeny is produced.

For example, the methods described herein can be used to provide a knock-in transgenic non-human mammal whose genome comprises a recombinant nucleic acid sequence comprising an α_{1A}-adrenergic receptor (AR) and a marker peptide operably linked to all or a functional portion of an α_{1A}-AR promoter, wherein the α_{1A}-AR and the marker peptide are expressed as a fusion protein in the transgenic non-human mammal. In this embodiment, the targeting construct is used to replace the endogenous α_{1A}-AR with an α_{1A}-AR-marker peptide fusion protein.

In a particular embodiment, the method comprises introducing a targeting construct which comprises the recombinant nucleic acid sequence comprising the α_{1A}-AR and the marker peptide, wherein the α_{1A}-AR and the marker peptide are expressed as a fusion protein, into a pronuclei of an embryo. The embryo can then be introduced into a pseudo-pregnant non-human female mammal under conditions in which the non-human female mammal gives birth to a chimeric transgenic non-human mammal whose genome comprises the recombinant nucleic acid sequence comprising the α_{1A}-AR and the marker peptide. The chimeric transgenic non-human mammal is bred with a second mammal to generate heterozygous F1 progeny that are heterozygous for the recombinant nucleic acid sequence comprising the α_{1A}-AR and the marker peptide. The heterozygous F1 progeny can then be crossbred under conditions in which a transgenic non-human mammal whose genome comprises a recombinant nucleic acid sequence comprising an α_{1A}-adrenergic receptor (AR) and a marker peptide, wherein the α_{1A}-AR and the marker peptide are expressed as a fusion protein in the transgenic non-human mammal, and homozygote F2 progeny is produced.

The invention also encompasses a transgenic non-human mammal produced by the methods described herein. In particular embodiment, the transgenic non-human mammals have constitutively active α_{1A}-ARs. In one embodiment, the invention is directed to a transgenic non-human mammals whose genome is homozygous for a recombinant nucleic acid sequence comprising an α_{1A}-AR and a marker peptide, wherein the α_{1A}-AR and the marker peptide are expressed as a fusion protein in the transgenic non-human mammal. In another embodiment, the invention is directed to a heterozygous transgenic non-human mammal whose genome is heterozygous for a recombinant nucleic acid sequence comprising an α_{1A}-AR and a marker peptide (F1 progeny). A particular characteristic of the heterozygous transgenic non-human mammals is that when their genome is made homozygous for the recombinant nucleic acid sequence comprising the α_{1A}-AR and the marker peptide, this results in a transgenic non-human mammal whose genome comprises a recombinant nucleic acid sequence comprising an α_{1A}-adrenergic receptor (AR) and a marker peptide, wherein the α_{1A}-AR and the marker peptide are expressed as a fusion protein in the transgenic non-human mammal (F2 progeny).

Thus the present invention is also directed to a method of producing a transgenic non-human mammal whose genome is heterozygous for a recombinant nucleic acid sequence comprising an α_{1A}-AR and a marker peptide. In one embodiment, the method comprises introducing a targeting construct which comprises the recombinant nucleic acid sequence comprising the α_{1A}-AR and the marker peptide, wherein the α_{1A}-AR and the marker peptide are expressed as a fusion protein, into a pronuclei of an embryo. The embryo can then be introduced into a pseudo-pregnant non-human female mammal under conditions in which the non-human female mammal gives birth to a chimeric transgenic non-human mammal whose genome comprises the recombinant nucleic acid sequence comprising the α_{1A}-AR and the marker peptide. The chimeric transgenic non-human mammal is bred with a second mammal to generate heterozygous F1 progeny that are heterozygous for the recombinant nucleic acid sequence comprising the α_{1A}-AR and the marker peptide.

Expression and/or overexpression of the α_{1A}-AR in the transgenic non-human mammal can be accomplished using a variety of methods. In one embodiment, a targeting construct for homologous recombination can be used.

One of skill in the art will easily recognize that the α_{1A}-AR-marker peptide fusion protein can be expressed or overexpressed in a number of different ways, any one of which may be used to produce the transgenic non-human mammals of the present invention. For example, a transgenic mammal according to the instant invention can be produced by the method of gene targeting. As used herein the term “gene targeting” refers to a type of homologous recombination which occurs as a consequence of the introduction of a targeting construct (for example, vector) into a mammalian cell (for example, an ES cell) which is designed to locate and recombine with a corresponding portion of the nucleic acid sequence of the genomic locus targeted for alteration (for example, overexpression, expression as a fusion protein) thereby introducing an exogenous recombinant nucleic acid sequence capable of conferring a planned alteration to the endogenous gene or introducing an exogenous nucleic acid which encodes a protein or portion thereof. As used herein an “exogenous nucleic acid sequence” refers to a nucleic acid sequence that is not normally found in a wild type mammal or cell, and thus is introduced into the mammal or cell. Thus, homologous recombination is a process by which a particular DNA sequence can be introduced. More specifically, regions of the targeting vector which have been genetically engineered to be homologous (e.g., complementary) to the endogenous nucleotide sequence of the gene which is targeted for expression (overexpression) or fusion, line up or recombine with each other such that the nucleotide sequence of the targeting vector is incorporated into (for example, integrates with) the corresponding position of the endogenous gene.

One embodiment of the present invention provides a vector construct designed to overexpress the α_{1A}-AR as a fusion protein with a marker peptide. In general terms, an effective targeting vector or construct for use in the compositions and methods of the present invention comprises a recombinant sequence that is effective for homologous recombination with the α_{1A}-AR gene.

Suitable targeting constructs of the invention can be prepared using standard molecular biology techniques known to those of skill in the art. For example, techniques useful for the preparation of suitable vectors are described by Maniatis, et al., Molecular Cloning: A Laboratory Manual, Cold Spring Harbor Laboratory, Cold Spring Harbor, N.Y. One of skill in the art will readily recognize that a large number of appropriate vectors known in the art can be used as the basis of a suitable targeting vector. In practice, any vector that is capable of accommodating the recombinant nucleic acid sequence required to direct homologous recombination and to express (overexpress) α_{1A}-AR as a fusion protein with a marker peptide can be used. For example, pBR322, pACY164, pKK223-3, pUC8, pKG, pUC19, pLG339, pR290, pKC101 or other plasmid vectors can be used. Alternatively, a viral or bacteriophage vector such as the lambda gt11 vector system can provide the backbone (for example, cassette) for the targeting construct.

Also provided are targeting constructs comprising all or a functional portion of an α_{1A}-AR promoter sequence, a marker peptide and/or all or a functional portion of α_{1A}-AR sequence, wherein the marker peptide and/or the α_{1A}-AR sequence are expressed under the control of (operably linked to) the α_{1A}-AR promoter sequence. In one embodiment, the construct comprises in a 5′ to 3′ direction about a 4.4 kb fragment of an α_{1A}-AR promoter sequence and an enhanced green fluorescent protein sequence. In a particular embodiment, the invention is directed to a targeting construct which comprises in a 5′ to 3′ direction about a 4.4 kb fragment of an α_{1A}-AR promoter sequence, an α_{1A}-AR sequence and an enhanced green fluorescent protein sequence. In another embodiment, the invention is directed to a targeting construct which comprises in a 5′ to 3′ direction about a 4.4 kb fragment of an α_{1A}-AR promoter sequence, an enhanced green fluorescent protein sequence and an α_{1A}-AR sequence.

According to techniques well known to those of skill in the art, genetically engineered (for example, transfected using electroporation or transformed by infection) ES cells, are routinely employed for the production of transgenic non-human embryos. ES cells are pluripotent cells isolated from the inner cell mass of mammalian blastocyst. ES cells can be cultured in vitro under appropriate culture conditions in an undifferentiated state and retain the ability to resume normal in vivo development as a result of being combined with blastocyst and introduced into the uterus of a pseudo-pregnant foster mother. Those of skill in the art will recognize that various stem cells are known in the art, for example AB-1, HM-1, D3. CC1.2, E-14T62a, RW4 or JI (Teratomacarcinoma and Embryonic Stem Cells: A Practical Approach, E. J. Roberston, ed., IRL Press).

It is to be understood that the transgenic non-human mammals described herein can be produced by methods other than the ES cell method described above, for example by the pronuclear injection of recombinant genes into the pronuclei of one-cell embryos or other gene targeting methods which do not rely on the use of a transfected ES cell, and that the exemplification of the single method outlined above is not intended to limit the scope of the invention to animals produced solely by this protocol.

The transgenic non-human mammals, and cell lines, primary tissue or cell cultures, cellular extracts or cell organelles isolated from the transgenic non-human mammals of the instant invention are useful for a variety of purposes. In one embodiment of the present invention the transgenic non-human mammals produced in accordance with the present invention are utilized as a source of cells for the establishment of cultures or cell lines (for example, primary, or immortalized), which are useful for the elucidation of α_{1A}-AR function. Such cells, which can be isolated from mammalian tissues, include neurons. The primary cell cultures, or cell lines, can be derived from any desired tissue or cell-type which normally express high levels of the α_{1A}-AR mRNA, including but not limited to neurons. Thus, the present invention is also directed to an isolated cell or cell line whose genome comprises a recombinant nucleic acid sequence comprising an α_{1A}-adrenergic receptor (AR) and a marker peptide, wherein the α_{1A}-AR and the marker peptide are expressed as a fusion protein in the cell.

The transgenic non-human mammals described herein can also be bred (for example, inbred, outbred or crossbred) with appropriate mates to produce colonies of animals whose genome comprises a recombinant nucleic acid sequence which comprises α_{1A}-adrenergic receptor (AR) and a marker peptide (e.g., EGFP), wherein the α_{1A}-AR and the marker peptide are expressed as a fusion protein in the transgenic non-human mammal. For example, the transgenic non-human mammals described herein can be crossbred with any commercially available disease mouse model (see, for example, The Jackson Laboratory JAX® mice) to explore the roles of α_{1A}-AR in the disease process. In one embodiment, the transgenic adenocarcinoma of mouse prostate (TRAMP) mouse can be crossbred with a transgenic mouse described herein to identify α_{1A}-AR cells in prostate cancer regulation (Gingrich, et al., Prostate Cancer and Prostatic Disease, 2:70-75 (1999)).

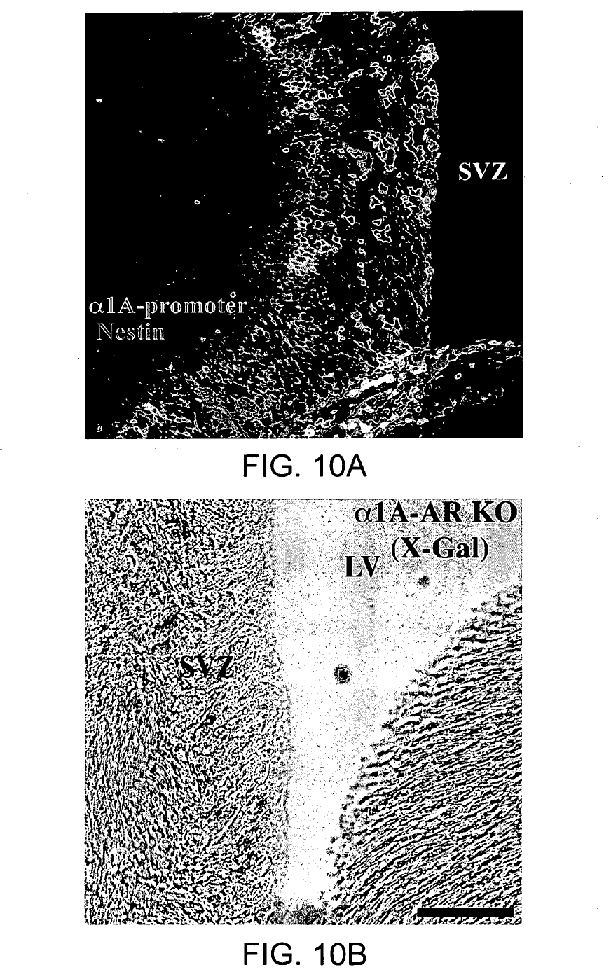

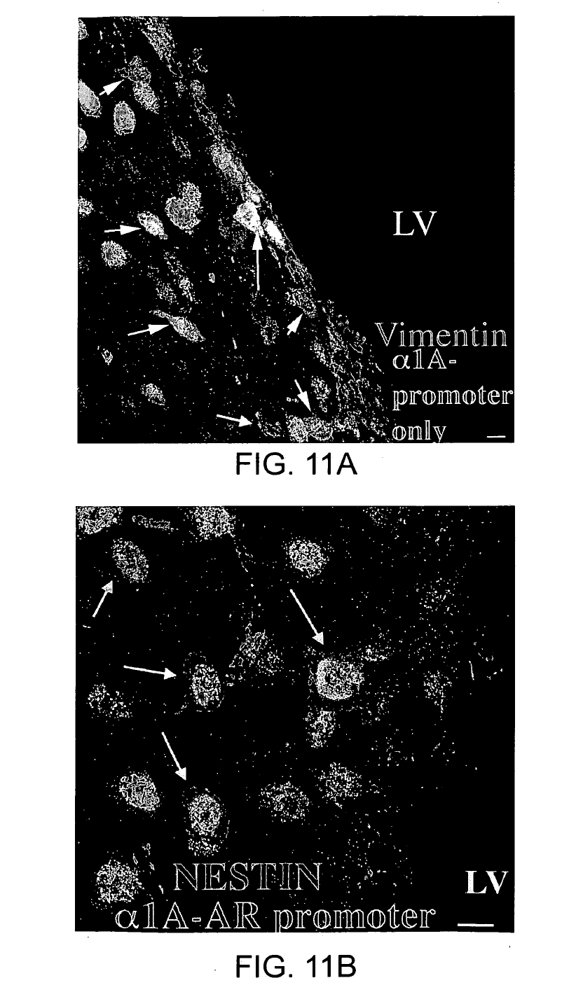

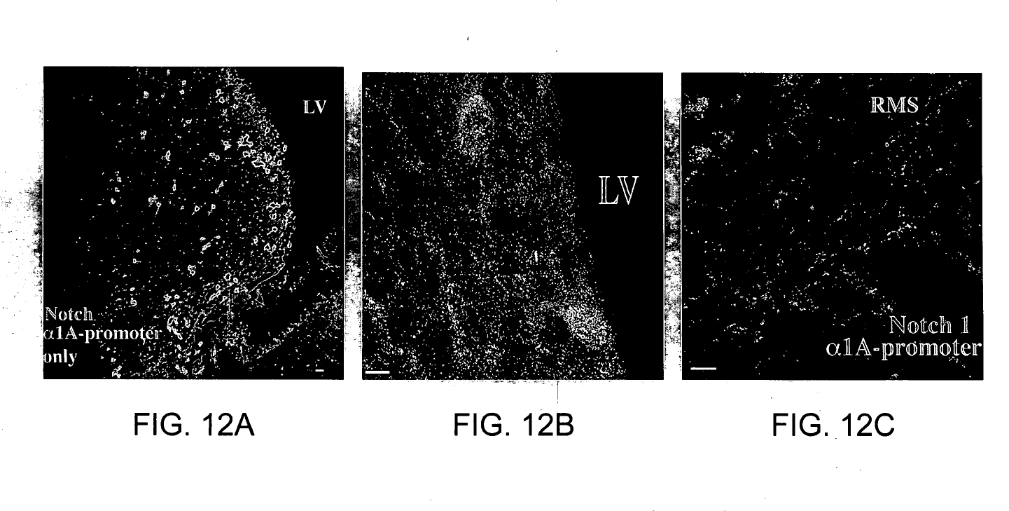

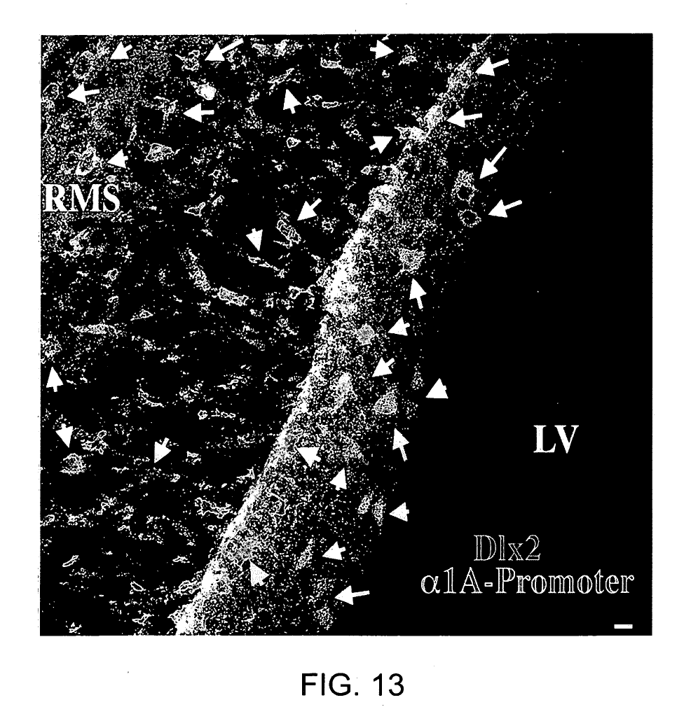

Localization studies in the brain using the EGFP-tagged transgenics, show that α_{1A}-ARs are expressed in neurons, GABAergic interneurons, some neurons that express NMDA receptors, and are also found in NG2-positive oligodendrocyte progenitors. EGFP expression is not seen in cerebral blood vessels, mature astrocytes or mature CC1-positive oligodenrocytes. However, this may be due to low abundance of the EGFP signal in these cell-types, rather than a lack of expression. These cell types are not mature neurons since it does not express the mature neuronal marker NEUN, nor it is an oligodendrocyte because it does not express the mature oligodendrocyte marker, CC1. The green α_{1A}-AR cells also did not co-localize with markers for young neurons, β-III tubulin, nor for young glia using S100 in the SVZ region but they do co-localize with these markers in the olfactory bulb. Since these SVZ cells are likely progenitors/stem cells, immunohistochemistry was performed using a series of markers associated with progenitors and neural stem cells. The α_{1A}-AR was expressed in the same location as a population of cells that also expressed nestin (FIG. 10A). This same pattern of cells in the subventricular zone was also seen in the α_{1A}-AR KO mice, stained blue with X-Gal (FIG. 10B). Higher magnification reveals that the promoter only EGFP is expressed in the cytoplasm, and that the α_{1A}-AR is expressed in the same cell as those marked with Vimentin (FIG. 11A) or Nestin (FIG. 11B). Also shown is that α_{1A}-AR expressing cells are found in the same region that expresses Notch 1 (FIGS. 12A-12C), which are expressed in NSCs. However, there is a small population of cells that co-express α_{1A}-AR and Notch 1 near the ventricular border (FIGS. 12A, 12B), but are likely expressed in neuroblasts of the RMS (FIG. 12C). Antibodies were also used against the transcription factor, D1x2, which labels TAP cells and neuroblasts. Some of the α_{1A}-AR expressing cells in the SVZ also expressed D1x2 but others did not (FIG. 13). It was noticed that α_{1A}-AR cells in the subventricular zone were of a mixed population; cells near the ependymal layers were rarely expressing D1x2, suggesting they are pre-TAP cells (FIG. 13), but α_{1A}-AR cells further inside from the ependymal layer expressed D1x2 (FIG. 13). The α_{1A}-AR cells also express D1x2 in the rostral migratory stream.

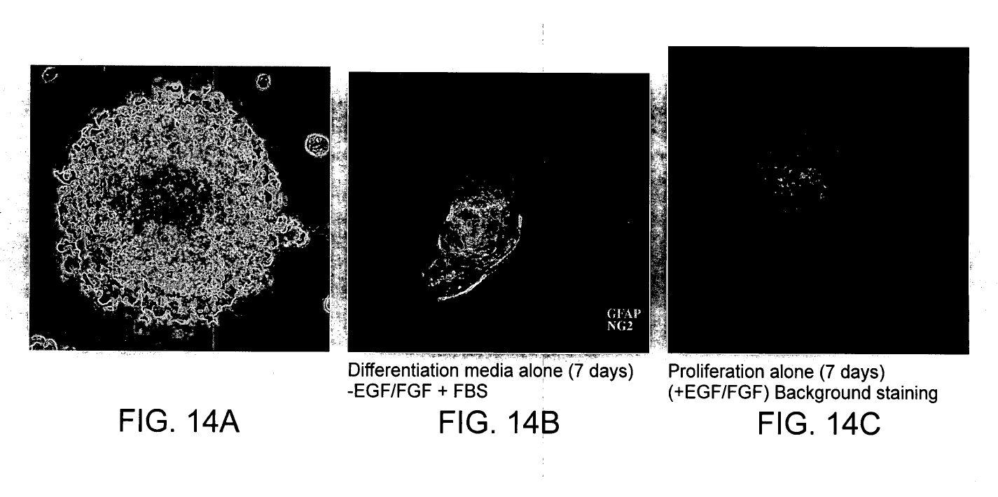

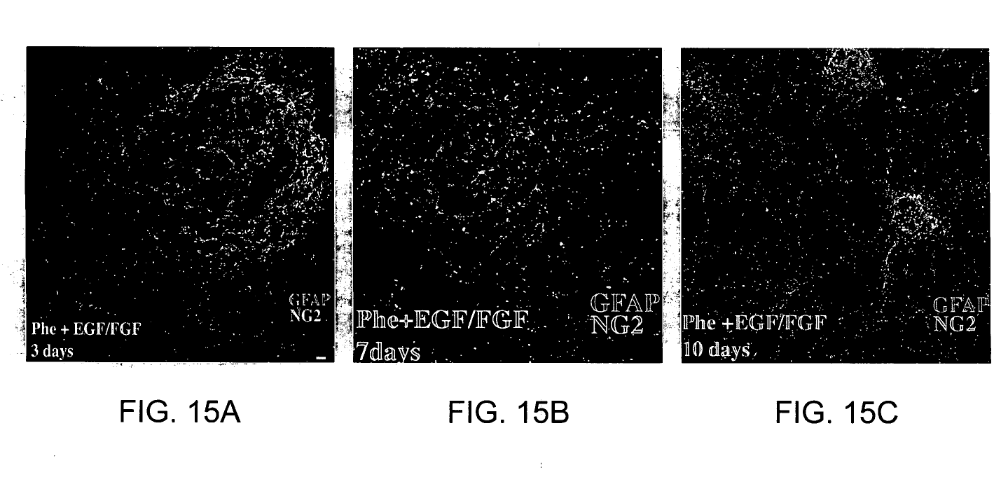

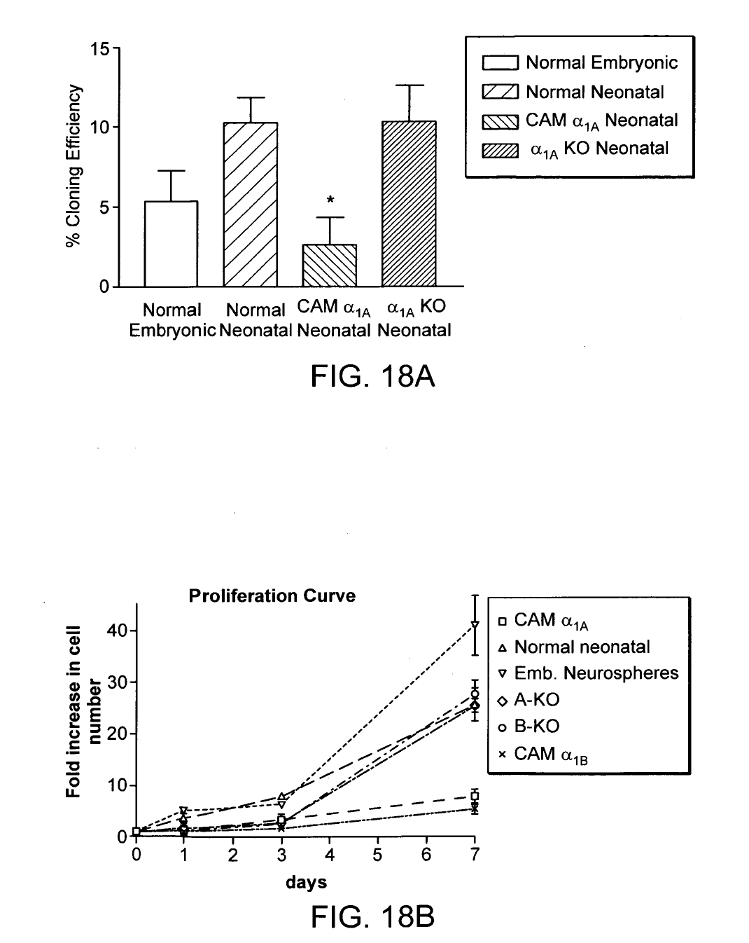

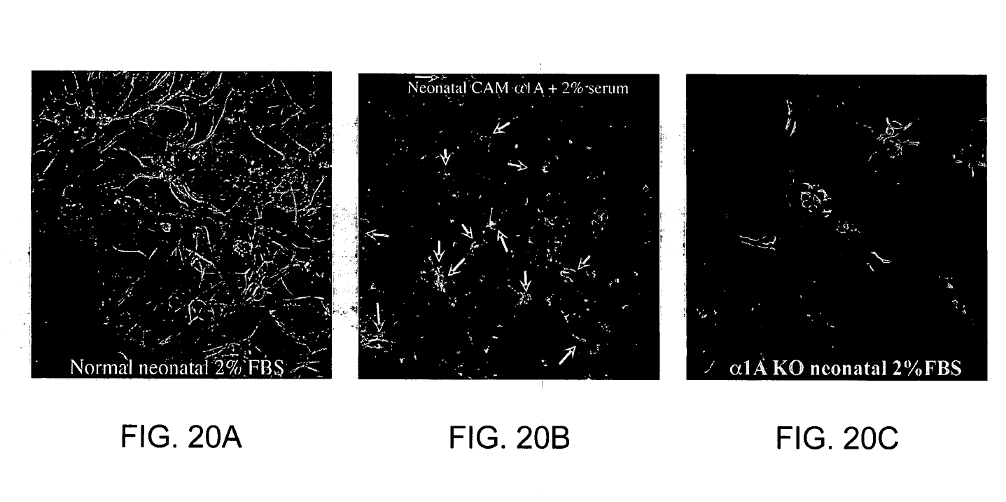

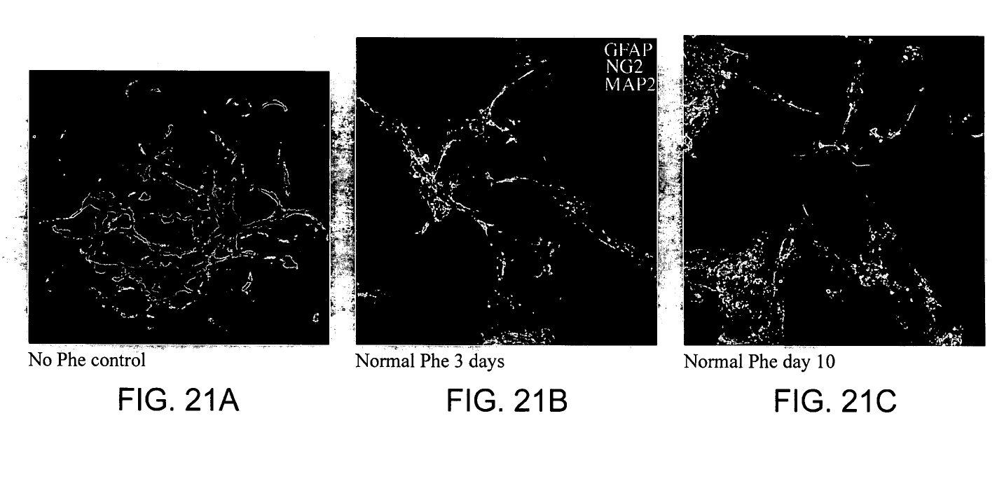

Neurospheres were isolated from normal embryonic or neonatal mice from isolated SVZ and were grown and passaged numerous times, and show that they can self-renew (FIG. 18A). When differentiation is induced by withdrawing EGF and FGF and adding serum for 7 days, EGF-responsive neurospheres can differentiate into MAP2-positive neurons (FIG. 14B), GFAP-positive astrocytes (FIG. 14B) or NG2-positive oligodendrocyte progenitors (FIG. 14B). Serum-induced differentiation of normal neonatal (FIG. 20A), CAM α_{1A} (FIG. 20B) or α_{1A}-KO (FIG. 20C) shows that these neurospheres have the potential to be pluripotent by differentiating into all three cell types, but the CAM α_{1A} had more expression of neurons and NG2 oligodendrocytes than normal cells, and was reversed by the α_{1A}-KO. Phenylephrine (α₁-AR agonist) was also added to a culture of normal mouse neurospheres when they were still in proliferation medium (+EGF/FGF). Phenylephrine also caused the differentiation of the neurospheres and seemed to enhance the rate of differentiation (FIGS. 15A-15C). By day 3 with phenylephrine, all three cell-types are present in neurospheres (FIG. 15A) but neurons and GFAP-positive cells dominate. By day 7-10, there appears to be a shift in the differentiation from GFAP-positive cells into NG2 (FIGS. 15B, 15C), as predicted from the types of cells expressing the α₁-ARs in the adult mouse brain (Papay, D M et al. J Comparative Neurology, 248:1-10 (2004). There also is likely a bipolar development of neurons and NG2 type cells (FIG. 15A). Similar data was obtained when neonatal, instead of embryonic, neurospheres from normal (FIGS. 21A-21C) or CAM α_{1A} (FIG. 20B) or α_{1A}-KO (FIG. 20C) were used.

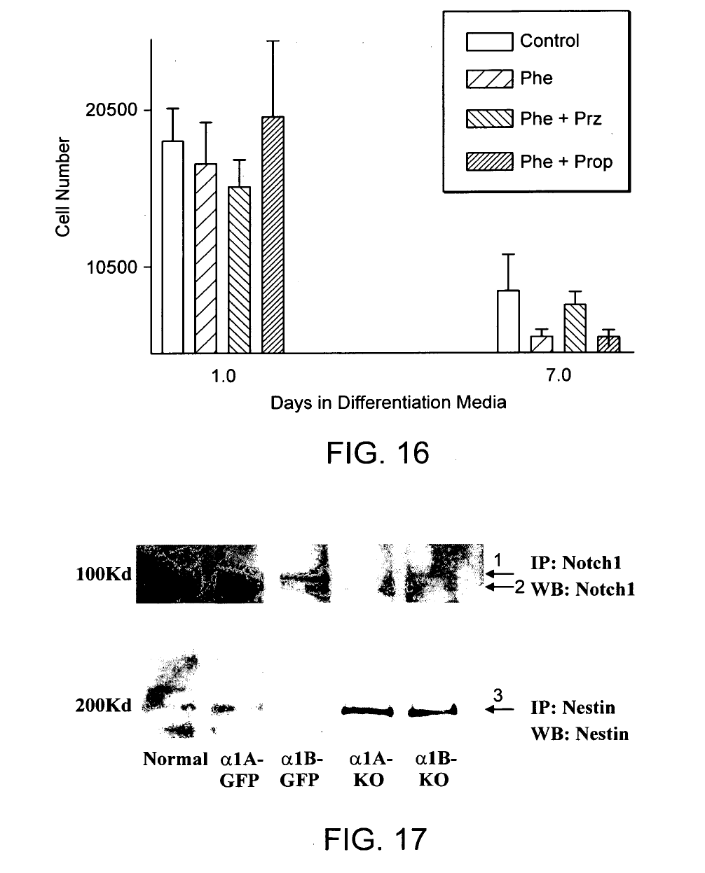

Differentiation rates (number of live cells) were also measured in normal neurospheres using differentiation media (−EGF/FGF) with and without phenylephrine. Phenylephrine enhances differentiation of neurospheres (FIG. 16), which would lead to a reduction in cell number. This effect can be blocked with prazosin (Prz), an α_{1A}-AR antagonist, but not with propranolol (prop), a β-AR antagonist, which indicates that α_{1A}-AR adrenergic influences on stem cell biology are specific. Variable results were received when phenylephrine was added to proliferation media (+EGF/FGF). It is likely that a mixed population of neurospheres is present, and depending upon what type gets seeded into the wells, produces the variable results. It appears that phenylephrine may cause both proliferation (FIG. 15A-15C) and enhance differentiation, depending upon the type of neurosphere.

Using isolated SVZ regions from adult mice and immunoprecipitation, it is shown herein that α₁-AR subtype signaling can regulate the protein levels of both Notch1 and Nestin (FIG. 17). Mature notch proteins are heterodimeric receptors derived from the cleavage of notch pre-proteins into an extracellular subunit and a transmembrane subunit including the intracellular region. Upon ligand binding by Delta or Serrate, Notch1 receptors are proteolitically cleaved, the intracellular domain of Notch (NICD) is released from the transmembrane segment and translocated to the nucleus, where it activates target genes. The relationship between α₁-ARs and Notch/Nestin appears inversely proportional, with the transmembrane (FIG. 17, arrow 1) and NICD (FIG. 17, arrow 2) cleaved signaling products of Notch 1 decreasing in the α₁-AR KO models. Conversely, protein levels of Nestin (FIG. 17, arrow 3) are increasing in the same samples. There also may be increased levels of the NICD fragment in the α_{1A}-AR GFP mice (with receptor overexpressed). This likely suggests that α₁-ARs modulate the transcription of Notch1 and Nestin. Because increased Notch-1 expression and cleavage releasing its intracellular domain (NICD) inhibit both dendrite growth and maturation, α_{1A}-AR signaling likely also modulates NSC function.

Thus, the present invention is also directed to a method of modulating (inhibiting, enhancing) Notch-1 and/or nestin activity (e.g., function, expression) of a cell that expresses Notch1 an/or nestin, comprising contacting the cell with an agent that modulates biological activity of α_{1A}-AR, expression of α_{1A}-AR or a combination thereof. In one embodiment, the method comprises inhibiting Notch-1 and/or nestin activity (e.g., function, expression) of a cell that expresses Notch1 and/or nestin, comprising contacting the cell with an agent that inhibits biological activity of α_{1A}-AR, expression of α_{1A}-AR or a combination thereof. In another embodiment, the method comprises enhancing Notch-1 and/or nestin activity (e.g., function, expression) of a cell that expresses Notch 1 and/or nestin, comprising contacting the cell with an agent that enhances biological activity of α_{1A}-AR, expression of α_{1A}-AR or a combination thereof. In a particular embodiment, the present invention is directed to a method of modulating Notch1 and/or nestin in an individual comprising administering to the individual an effective amount of an agent that modulates biological activity of α_{1A}-AR, expression of α_{1A}-AR or a combination thereof.

It is shown herein that the α_{1A}-AR is being expressed and regulates differentiation of TAP cells into neuroblasts and oligodendrocyte progenitors and is a major player in the maturation process; but also that an α_{1A}-AR expressing cell type exists in the SVZ that is pre-TAP and is likely a type of NSC. Thus, the present invention is also directed to a cell or cell line (isolated cell or cell line) that is α_{1A}-AR+, Nestin+, D1x2−, and Notch 1−, and likely includes other markers such as one or more of the markers listed in Table 1. In a particular embodiment, the cell or cell line is isolated. As used herein, a composition (e.g., a cell) is isolated (pure, substantially pure) when it is substantially free of cellular material, when it is isolated from non-recombinant cells, or free of chemical precursors or other chemicals when it is chemically synthesized.

The transgenic non-human mammals can also be used in a variety of methods. In one embodiment, the present invention is directed to a method of identifying an agent that modulates (enhances, inhibits) α_{1A}-AR (e.g., the biological activity, function and/or expression of α_{1A}-AR). The method comprises administering the agent to a transgenic mouse or a cell isolate whose genome comprises a recombinant nucleic acid sequence which comprises α_{1A}-adrenergic receptor (AR) and a marker peptide (e.g., an enhanced green fluorescent protein (EGFP)), wherein the α_{1A}-AR and the marker peptide are expressed as a fusion protein in the transgenic mouse. Whether α_{1A}-AR is modulated in the transgenic mouse or in the cell isolate is compared to a control mouse or cell, wherein if α_{1A}-AR is modulated in the transgenic mouse or cell isolate compared to the control mouse or cell, then the agent modulates α_{1A}-AR. Methods of determining whether α_{1A}-AR is modulated are provided herein, are known in the art, and include determining whether the agent modulates expression and/or one or more biological functions of α_{1A}-AR. Biological function of α_{1A}-AR include modulating neural stem cell differentiation; regulating differentiation or proliferation of neural stem cells or progenitor cells; enhancing expression of neural stem cells such as TAP cells, neuroblasts, oligodendrocyte progenitors; inhibiting production of astrocytes; enhancing cognitive function.

In a particular embodiment, the method includes determining whether the agent modulates (enhances, inhibits) the α_{1A}-AR activity of enhanced cognitive function such as in a transgenic non-human animal described herein. This method can further comprise determining whether the agent modulates (enhances, inhibits) the α_{1B}-AR activity of enhanced cognitive function. Methods for determining cognitive ability are provided herein (e.g., dry maze test, Morris water maze test) and are known to those of skill in the art.

The present invention also provides a method of identifying an agent that modulates (enhances, inhibits) neurogenesis. In a particular embodiment, the present invention provides a method of identifying an agent that modulates neural stem cell or progenitor cell differentiation or proliferation by α_{1A}-AR. As used herein, a “neural stem cell” refers to a stem cell found in neural tissue that can give rise to TAP cells and neurons, astrocytes and oligodendrocytes (pluripotency); and a “progenitor cell” refers to a dividing cell with a restrictive capacity to differentiate (e.g., a putative stem cell in which self-renewal has not yet been demonstrated). The method comprises administering the agent to a transgenic non-human mammal or a cell isolate whose genome comprises a recombinant nucleic acid sequence which comprises α_{1A}-adrenergic receptor (AR) and a marker peptide (e.g., EGFP), wherein the α_{1A}-AR or α_{1B}-AR and the marker peptide are expressed as a fusion protein in the transgenic non-human mammal. Whether expression of one or more neural stem cell markers (e.g., α_{1A}-AR; α_{1B}-AR) is modulated in the transgenic non-human mammal or in the cell isolate compared to a control non-human mammal or cell is determined, wherein if the expression of the one or more neural stem cell marker is modulated in the transgenic non-human mammal or cell isolate compared to the control mouse, then the agent modulates neural stem cell or progenitor cell differentiation or proliferation by α_{1A}-AR. Therefore, after the agent is administered to the transgenic non-human mammal or contacted with the cell (cells isolated from the animal), the generation, migration, proliferation and/or incorporation of newly generated neural cell types (or the lack of generation, migration, proliferation and/or incorporation of newly generated neural cell types) can be monitored (detected using the fluorescent protein).

Any suitable control can be used in the methods of the present invention. For example, a suitable control is a wild type non-human mammal. In a particular embodiment, the control non-human mammal is of the same species as the transgenic non-human mammal used in the method.

Also encompassed by the present invention are agents identified in the methods of the present invention.

The present invention is also directed to a method of determining whether a cell is a neural stem cell comprising identifying markers expressed on the cell, wherein if the marker comprises α_{1A}-AR, α_{1B}-AR, β-AR, nestin, notch 1, vimentin and glia fibrillary acidic protein (GFAP), then the cell is a neural stem cell.

A variety of methods for identifying markers expressed on a cell are known in the art. For example, fluorescence activated cell sorting (FACS) analysis or methods based upon antibody approaches can be used. In addition, α_{1A}-AR or α_{1B}-AR RNA can be identified using PCR methods.

A method of regulating differentiation and/or proliferation of a neural stem cell and/or progenitor cell is also encompassed by the present invention. The method comprises contacting the neural stem cell or progenitor cell with an agent that modulates biological activity of α_{1A}-AR, expression of α_{1A}-AR or a combination thereof, in the neural stem cell or progenitor cell. In one embodiment, the differentiation and/or proliferation of the neural stem cell or progenitor cell is enhanced comprising contacting the neural stem cell or progenitor cell with an agent that enhances biological activity of α_{1A}-AR, expression of α_{1A}-AR or a combination thereof, in the neural stem cell or progenitor cell. In another embodiment, the neural stem cell differentiates into one or more cells selected from the group consisting of: a transiently amplifying progenitor (TAP) cell, a neuroblast, an NG2 oligodendrocyte and a combination thereof. In yet another embodiment, the differentiation and/or proliferation of the neural stem cell or progenitor cell is inhibited comprising contacting the neural stem cell or progenitor cell with an agent that inhibits biological activity of α_{1A}-AR, expression of α_{1A}-AR or a combination thereof, in the neural stem cell or progenitor cell.

In a particular embodiment, the present invention is directed to a method of regulating differentiation or proliferation of a neural stem cell or progenitor cell in an individual comprising administering to the individual an effective amount of an agent that modulates biological activity of α_{1A}-AR, expression of α_{1A}-AR or a combination thereof. In one embodiment, the individual can be administered the agent that modulates biological activity of α_{1A}-AR, expression of α_{1A}-AR or a combination thereof directly (an in vivo approach). In another embodiment, cells (e.g., neural cells which express α_{1A}-AR) of the individual can be removed and contacted with the agent that modulates biological activity of α_{1A}-AR, expression of α_{1A}-AR or a combination thereof, and the cells are then maintained under conditions in which the biological activity of α_{1A}-AR, expression of α_{1A}-AR or a combination thereof is modulated in the presence of the agent. The cells can then be administered to the individual (an ex vivo approach). The methods described herein can be used, for example, to treat an individual with a neurodegenerative disorder (e.g., Parkinson's Disease; Alzheimer's Disease), a cognitive defect and/or a neural injury (e.g., head trauma, spinal chord injury). In addition, the methods described herein can used in combination with other treatments/agents used to treat such disease/conditions (e.g., as part of a cocktail of treatments).

As shown in the exemplification, α_{1B}-AR or β-AR also plays a role in neurogenesis. Thus, the methods described herein can further comprise contacting the neural stem cell or progenitor cell with an agent that modulates biological activity of α_{1B}-AR or β-AR, expression of α_{1B}-AR or β-AR or a combination thereof, in the neural stem cell or progenitor cell.

The present invention also relates to methods of treatment or prevention of conditions or diseases associated with abnormal expression and/or function of α_{1A}-AR in an individual. In one embodiment, the present invention is directed to a method of treating a neurodegenerative disorder in an individual in need thereof, comprising administering to the individual an agent that regulates biological activity of α_{1A}-AR, expression of α_{1A}-AR or a combination thereof, in the individual. Examples of neurodegenerative disorders include Alzheimer's Disease, Parkinson's Disease, Multiple System Atrophy, cognitive disorders and spinal cord injuries.

Also encompassed by the present invention are methods of modulating (e.g., enhancing, inhibiting) cognitive function (e.g., learning, memory) in an individual in need thereof comprising administering to the individual an agent that modulates biological activity of an α₁-AR, expression of an α₁-AR or a combination thereof, in the individual. In particular embodiments, the invention is directed to methods of enhancing cognitive function (e.g., learning, memory) in an individual comprising administering to the individual an agent that enhances biological activity of an α₁-AR, expression of an α₁-AR or a combination thereof, in the individual. The α₁-AR that is modulated (e.g., enhanced) in the methods include α_{1A}-AR, α_{1B}-AR and/or a combination thereof. In a particular embodiment, an α_{1A}-AR selective agonist is used. As shown herein “cognitive function” includes learning and memory.

The present invention also provides for methods of culturing stem cells and/or progenitor cells (e.g., adult stem or progenitor cells) in order to obtain neurons (e.g., interneurons, stimulatory glutamatergic neurons) that are to be implanted (reimplanted) into an individual in need thereof. In particular embodiments, the stem cells and/or progenitor cells are contacted (cultured) with a ligand of α_{1A}-AR, a ligand of β-AR, an agent that modulates (e.g., enhances) that function and/or expression of α_{1A}-AR and/or β-AR or a combination thereof. The cells are maintained under conditions in which neurons are produced. The neurons are then administered (implanted) into the individual (e.g., systemically, at a particular site such as the brain or spinal chord of an individual). The cells that are cultured can be obtained from the individual in need of treatment or from another individual.

The agent for use in the methods of the present invention can be for example, a nucleic acid molecule (e.g., DNA, RNA, anti-sense DNA, anti-sense RNA, interfering RNA (e.g., siRNA, shRNA)), a protein, a peptide, a polypeptide, a glycoprotein, a polysaccharide, an organic molecule, an inorganic molecule, a fusion protein, etc. Particular examples of agents which can be used in the methods of the present invention include agonists, antagonists and antibodies or antigen binding fragments thereof which selectively bind an α₁-AR (e.g., α_{1A}-AR; α_{1B}-AR; β-AR).

The agents can be administered together with other components of biologically active agents, such as pharmaceutically acceptable surfactants (e.g., glycerides), excipients (e.g., lactose), stabilizers, preservatives, humectants, emollients, antioxidants, carriers, diluents and vehicles. If desired, certain sweetening, flavoring and/or coloring agents can also be added. The agents can be formulated as a solution, suspension, emulsion or lyophilized powder in association with a pharmaceutically acceptable parenteral vehicle. Examples of such vehicles are water, saline, Ringer's solution, isotonic sodium chloride solution, dextrose solution, and 5% human serum albumin. Liposomes and nonaqueous vehicles such as fixed oils can also be used. The vehicle or lyophilized powder can contain additives that maintain isotonicity (e.g., sodium chloride, mannitol) and chemical stability (e.g., buffers and preservatives). The formulation can be sterilized by commonly used techniques. Suitable pharmaceutical carriers are described in Remington's Pharmaceutical Sciences.

The agents can be administered to a host in a variety of ways. Potential routes of administration include intradermal, transdermal (for example, utilizing slow release polymers), intramuscular, intraperitoneal, intravenous, inhalation, subcutaneous or oral routes. Any convenient route of administration can be used, for example, infusion or bolus injection, or absorption through epithelial or mucocutaneous linings. The agent can be administered in combination with other components such as pharmaceutically acceptable excipients, carriers, vehicles or diluents.

In the treatment methods of treatment, an “effective amount” of the agent is administered to an individual. As used herein the term “effective amount” an amount that results in amelioration or prevention of the neurodegenerative disease in the host (e.g., results in a significant, such as a statistically significant difference in the neurodegenerative disease in the host). The amount of agent required to treat the neurodegenerative disease will vary depending on a variety of factors including the size, age, body weight, general health, sex and diet of the host as well as the time of administration, and the duration or stage of the particular condition or disease which is being treated. Effective dose ranges can be extrapolated from dose-response curves derived in vitro or an in vivo test system which utilizes the transgenic non-human mammals described herein.

TABLE 1
Markers for different likely SVZ cell types.
Markers

Stem cell/TAP
Nestin
Vimentin
GFAP (polyclonal)
GFAP (monoclonal)
PSA-NCAM
EGF-R
FGF2-R
Notch 1
Dlx2
mCD24
Early neuronal
MAP2
β III Tubulin
Glial
S100
NG2
GFAP

### EXEMPLIFICATION

### Example 1

### Localization of α_{1A}-AR

### Materials and Methods

Reagents. The agents and their sources used in this study were: 6-fluoronorepinephrine (6FNE) hydrochloride (Sigma-Aldrich, St. Louis, Mo., USA); isoflurane (Abbott Laboratories, North Chicago, Ill., USA). 2-[β-(4-hydroxyl-3-¹²⁵I-iodophenyl)ethylaminomethyl]-tetralone (¹²⁵I-HEAT) (Perkin Elmer, Inc. Massachusetts, USA). All reagents used to make the artificial cerebrospinal fluid (ACSF) were from J.T. Baker, Inc. (Phillipsburg, N.J., USA). All other reagents were from Fisher Scientific (Pittsburgh, Pa., USA).

Animal use. Mice were housed and provided veterinary care in an AAALAC-accredited animal care facility. This investigation conforms to the Guide for the Care and Use of Laboratory Animals published by the US National Institutes of Health (NIH publication No. 8523, revised 1996) and was approved by the Animal Research Committee of The Cleveland Clinic Foundation and the University of North Dakota. α_{1A}-AR knockout (KO) mice were backcrossed onto C57 for 5 generations. α_{1A}-AR transgenic mice were on a B6/CBA background. Mice that are 1-4 months old were used for immunohistochemistry and X-Gal analysis.

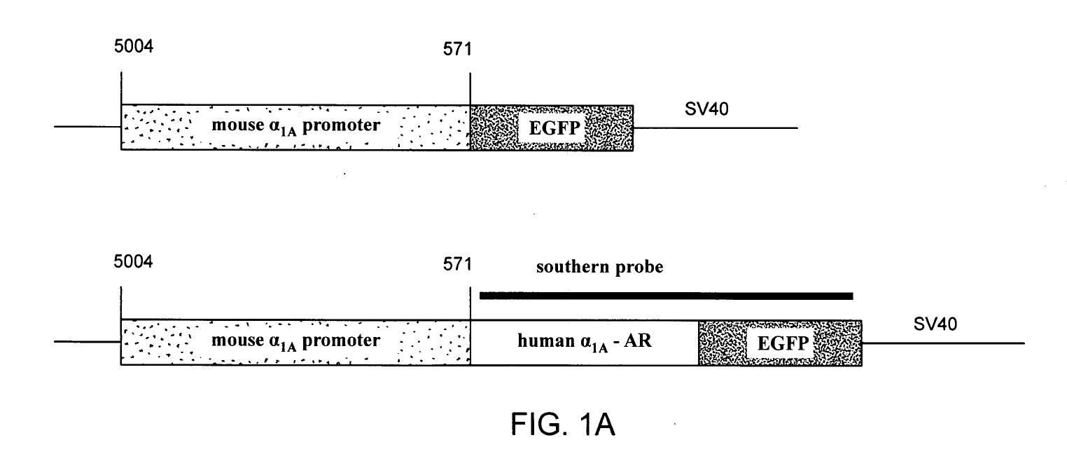

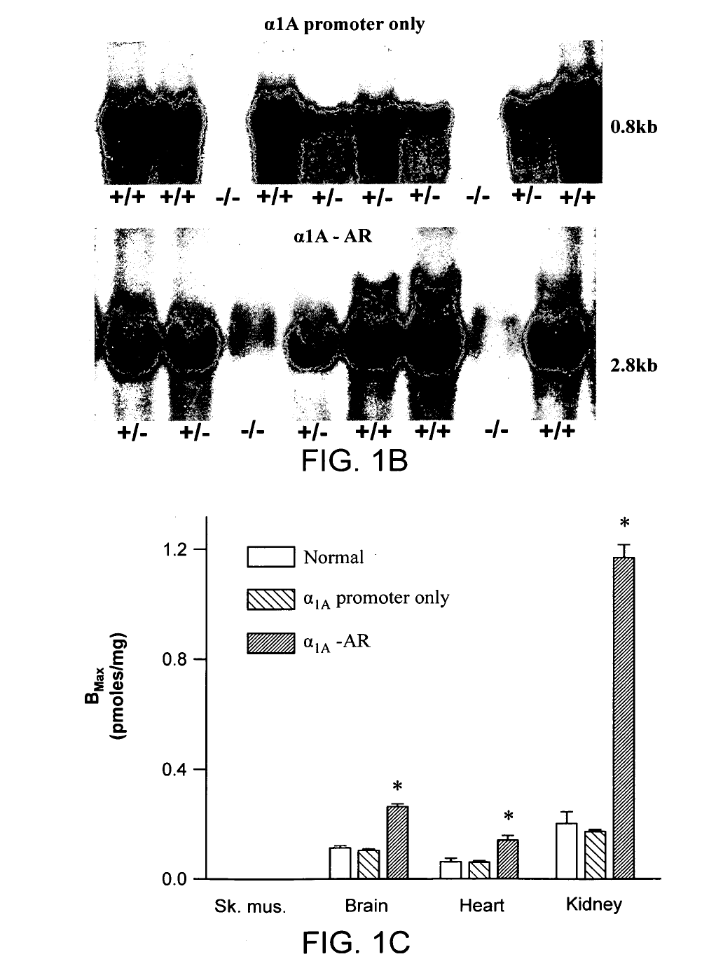

Transgene Construction and Genotyping. The transgene was constructed as previously described (Rorabaugh et al., Cardiovas. Res., 65:436-445 (2005)) except for the use of the human α_{1A}-AR-tagged EGFP cDNA (a gift from Gozoh Tsujimoto, Kyoto University, Kyoto, Japan), which was previously characterized in vitro (Hirasawa et al., Mol. Pharmacol., 52:764-770 (1997)). Two constructs were generated: One construct contained a 4.4 kb fragment of the mouse α_{1A}-AR promoter (O'Connell et al., Mol. Pharmacol., 59:1225-1234 (2001); Rorabaugh et al., Cardiovas. Res., 65:436-445 (2005)) to drive expression of a cDNA that encodes the human α_{1A}-AR tagged at the end of the C-tail with EGFP (FIG. 1A). The other construct contained the same mouse α_{1A}-AR promoter to drive expression of the EGFP alone without the receptor attached (FIG. 1A). The Case Western Reserve University Transgenic Core facility injected approximately 200 copies of each transgene into the pronuclei of one-cell B6/CBA mouse embryos, which were surgically implanted into pseudo-pregnant female mice. Three founder mice were identified and subsequent generations were genotyped by southern analysis of genomic DNA. Copy number as assessed by southern blot intensity was similar between the lines. The three lines were screened for EGFP expression by immunohistochemistry and found to be similar in intensity and localization. One male line was chosen and F2 mice were mated to homozygosity. Data is derived from several homozygous mice but from one founder line. Control mice are normal B6/CBA, which are bred independently from the transgenic lines to ensure purity.

Ligand Binding. Membranes were prepared and ligand binding was performed as previously described (Rorabaugh et al., Cardiovas. Res., 65:436-445 (2005)). Saturation binding used the nonselective ₁-AR antagonist, 2-[β-(4-hydroxyl-3-¹²⁵I-iodophenyl)ethylaminomethyl]-tetralone (¹²⁵I-HEAT) as the radioligand, using a K_{D} amount (100 pM) to label total α₁-ARs and not allowing more than 10% of the total tracer to be bound. Bₘₐₓ and K_{D} were determined through the use of Graphpad Prism. Statistical analysis of the binding data used a one-way ANOVA followed by a Newman-Keul's post-test where p<0.05 was considered significant.

β-Galactosidase activity. α_{1A}-AR knockout (KO) mice were designed with replacement of the first exon of the α_{1A}-AR with the Escherichia Coli β-galactosidase gene LacZ (Rokosh and Simpson, Proc. Natl. Acad Sci, USA, 99:9474-9479 (2002)). To localize β-galactosidase expression in situ in the α_{1A}-AR KO brain and compare to our mouse model, α_{1A}-AR KO mice anesthetized with isoflurane were perfused through the cardiac apex for 10 min with PBS (pH 7.3), perfusion-fixed for an hour at room temperature with 4% paraformaldehyde in PBS (pH 7.3), and perfusion-rinsed with PBS at 4° C. (Rokosh & Simpson, Proc. Natl. Acad Sci, USA, 99:9474-9479 (2002)). After washing, the brain and spinal cord was dissected out and placed in 10 ml of staining solution containing 1 mg/ml X-gal/5 mM K₃Fe(CN)₆/5 mM K₄Fe(CN)₆.3H₂0/2 mM MgCl₂/0.02% Nonidet P-40/0.01% deoxycholate in PBS and 40 mM Tris base (pH7.3) and incubated for 36 hours at room temperature. The blue-stained tissue was postfixed in 10% buffered formalin as previously described (Rokosh and Simpson, Proc. Natl. Acad Sci, USA, 99:9474-9479 (2002)). Samples were placed on a vibrotome, sliced at 50 microns, and mounted on coverslips.

Immunohistochemistry. Mice were injected with 0.1 ml heparin at 1000 U/ml i.p. and 0.1 ml Nembutal (50 mg/ml) i.p. Mice were perfused with 15 ml ice-cold heparin solution (10 U/ml) in PBS through the apex of the heart. This was followed by perfusion of 100 ml of 4% paraformaldehyde in phosphate buffer. The brain was removed and placed in 4% paraformaldehyde overnight at 4° C. The solution was replaced with cryoprotection solution (20% glycerol, 20% 0.4M Sorensen's Buffer, pH 7.6) overnight or longer at 4° C. Brains were embedded in 2-3% agarose in PBS after washing three times in PBS. 50-75 micron sections were cut using the Leica VT 1000S vibrotome. Sections were put into cryoprotection solution (20% glycerol, 20% ethylene glycol, 60% PBS) and stored at 4° C. until ready for use.

Immunohistochemistry was performed in 24-well plates by a free-floating method. Specimens were first washed 3 times for 5 minutes in PBS, followed by incubation with blocking buffer (6% BSA+0.3% Trition X-100 in PBS) for 1 hour at room temperature on a shaker. The blocking buffer was removed and then replaced with the respective primary antibodies made up in blocking buffer for typically a 2 day incubation at 4° C. on a shaker. Next, the specimens were washed 3 times for 5 minutes in PBS, and then incubated for 1 hour at room temperature on a shaker with the respective secondary antibodies made up in blocking buffer. This was followed by 3 washes again for 5 minutes in PBS followed by two rinses in distilled water. The specimens were then incubated with 10 mM copper sulfate in 50 mM ammonium acetate for 1 hour at room temperature on a shaker to reduce autofluorescence (Schnell, Sa et al., J. Histochem. Cytochem., 47:719-730 (1999)). Finally, they were rinsed twice with distilled water and mounted with Vectashield Mounting Media with DAPI (Vector Laboratories, Burlingame, Calif., Cat. #H-1200).

Cell types were identified with previously characterized antibodies. Mature oligodendrocytes with mouse anti-CC1 (Oncogene Research Products, Inc. at 1:100) (Messersmith et al., 2000) and oligodendrocyte precursers with rabbit anti-NG2 (a gift from William Stallcup, Burham Inst, La Jolla at 1:4000). Specificity of the NG2 antibody has been reported (Stallcup, W B, et al., Dev. Biol., 831:154-165 (1981)). Astrocytes were identified with mouse anti-GFAP (Chemicon International, Temecula, Calif. at 1:2500) (Debus, E., et al., Differentiation, 25:193-203 (1983)). Neurons were identified with mouse anti-NEUN (Chemicon at 1:3000) (Mullen, R J., et al. Development, 116:201-211 (1992)). GABA containing neurons were identified with rabbit anti-GABA (Abcam, Cambridge, UK at 1:500) (Somogyi, P, et al., J. Neurosci., 4:2590-2603 (1984)). GAD containing neurons were identified with goat anti-GAD 65/67 (Santa Cruz, Santa Cruz, Calif. at 1:50) (Rubio-Aliaga, I., et al., J. Biol. Chem., 279:2754-2760 (2004)). NMDA receptor containing neurons were identified rabbit anti-NR1 (Chemicon, at 5 ug/ml) (Petralia, R., et al., J. Neurosci., 14:667-696 (1994)) and α_{1A}-AR tagged receptors or EGFP alone with rabbit anti-GFP (Abcam at 1:2500) (Dubois-Dauphin, M., et al., J. Comp. Neurol., 474:108-122 (2004)). Presynaptic vesicles were labeled with sheep anti-α-synuclein (Chemicon, at 1:500) (Gai, W P, et al., J. Neurochem., 73:2093-2100 (1999)). Bound primary antibodies were detected by incubation with the appropriate FITC-coupled or Rhodamine-coupled secondaries (Jackson ImmunoResearch, or Molecular Probes) from 1:1000-3000 for fluorescent studies.

Confocal Microscopy. Sections were analyzed on a confocal laser-scanning microscope (Model Aristoplan; Leica, Inc., Deerfield, Ill.). Confocal images represent optical sections of 2-3 micron axial resolution and an average of 6 line-scans. Fluorescence in red, green, and blue channels was collected simultaneously. In the red/green merge images, areas of co-localization are yellow. For images under very low magnification, it was impossible to perform confocal analysis. Therefore, in these circumstances, we used fluorescent microscopy.

Photomicrograph Production. Confocal and fluorescent images were saved as tiff files at maximal resolution. Brightness settings as well as image sizing and lettering were performed with the use of Adobe Photoshop software.

Slice Preparation and Cell Identification. Hippocampal brain slices were prepared from α_{1A}-AR-EGFP mice in strict accordance with a protocol approved by the Institutional Animal Care and Use Committee at the University of North Dakota. Mice were deeply anesthetized with isoflurane, sacrificed by decapitation, and their brains rapidly removed and placed in an ice-cold saline solution containing (in mM): 130 NaCl, 24 NaHCO₃, 5 KCl, 1.25 NaH₂PO₄, 0.5 CaCl₂, 4 MgCl₂ and 10 glucose (saturated with 95% O₂-5% CO₂). The brain was then sectioned at 250-350 μm thick intervals using a Microm HM 650V vibrating blade microtome (Richard-Allen Scientific, Kalamazoo, Mich.). Slices were incubated at 35° C. for 40 min and then allowed to recover for at least 30 min at room temperature (22±1° C.) before experimentation. Slices were transferred to a PC-H chamber with a coverslip bottom (SD Instruments, Grants Pass, Oreg.) mounted on the fixed stage of an upright microscope (Olympus BX51 WI, Tokyo, Japan) equipped with a filter set for fluorescence imaging of EGFP and IR-DIC optics. Interneurons were visualized using 470 nm and 900 nm light with a water immersion objective (Olympus LUMPlanF1 60×IR) and two CCD cameras (Olympus MagnaFire and Sony XC-75, Tokyo, Japan). Dedicated MagnaFire software and a Hamamatsu C-2400 camera controller (Hamamatsu City, Japan) were used to adjust image quality in GFP and IR-DIC channels, respectively. During experimentation, slices were continuously superfused at 2-4 cc/min with ACSF containing (in mM): 130 NaCl, 24 NaHCO₃, 5.0 KCl, 1.25 NaH₂PO₄, 2.5 CaCl₂, 1.5 MgCl₂, and 10 glucose (saturated with 95% O₂-5% CO₂).

Electrophysiological Recordings. Cell-attached patch recordings were made from EGFP-expressing hippocampal CA1 interneurons using previously described techniques (Bergles, D E, et al., Nature, 405:187-191 (1996)). Action potentials (APs) were recorded using an Axopatch 200B amplifier (Axon Instruments, Union City, Calif.) in voltage-clamp mode, filtered at 2 kHz, digitized at 10 kHz (Digidata 1322A; Axon Instruments), and recorded to disk using pClamp9.0 software (Axon Instruments). The internal solution used for these recordings contained (in mM): 135 KMeSO₄, 8 NaCl, 10 HEPES, 2 MgATP, 0.1 BAPTAK₄, pH 7.2. Pipettes were pulled using a two-stage puller (Narishige PP-83, Tokyo, Japan), and had resistances of 4-5 MΩ. EGFP-expressing interneurons were approached with slight positive pressure. After contact, slight negative pressure was applied and seals (2-10 GΩ) formed slowly with negative holding current. For these recordings, the patch was held at 0 mV (pipette potential=−60 mV). Experiments were performed at 22±1° C. To minimize oxidation of the selective α-AR agonist 6FNE (Kirk et al., 1979), stock solutions were made fresh and added to oxygenated saline just before their application and high perfusion rates (2-4 cc/min) were maintained. Data were analyzed offline using Origin (OriginLab Corp., Northampton, Mass.) and Mini analysis 6.0.1 (Synaptosoft Inc., Decatur, Ga.). Each detected event was inspected visually to exclude obvious false action potentials (AP) before analysis. Frequency histograms were constructed by averaging the number of APs in 60 sec. Statistical significance (p<0.05) was determined using the paired Student's t test, and mean values are expressed ±SEM.

### Results

The constructs used for injection into the pronuclei of B6/CBA mouse embryos are shown in FIG. 1A. The constructs used the human cDNA for the α_{1A}-AR, which is fused in frame, at the end of the coding region for the α_{1A}-AR, with the cDNA for the EGFP (Hirasawa, A., et al., Mol. Pharmacol., 52:764-770 (1997)). The human α_{1A}-AR has the exact pharmacological and functional profile as the mouse α_{1A}-AR but was used as a means of genetic identification from nontransgenic mice. A construct with the same mouse α_{1A}-AR promoter that would drive the expression of the EGFP cDNA alone without the attached α₁-AR cDNA was also produced. This was to study effects due to receptor regulation and, without downregulation and turnover of the receptor, would likely allow greater visualization of the EGFP signal. Heterozygotes and homozygotes were identified by probing EcoR1-digested tail-snipped genomic DNA, transferred onto nitrocellulose, and hybridized with a radioactive probe containing the entire human α_{1A}-AR-EGFP cDNA. The promoter-only EGFP mice were identified by an 0.8 kb band, while α_{1A}-AR-EGFP mice displayed a 2.8 kb band (FIG. 1B). Again, as for the α_{1B}-AR-EGFP mice (Papay, R., et al., Comp. Neurol., 478:1-10 (2004)), an antibody against EGFP was needed to enhance the EGFP signal, due to the low copy number insertion and weak expression of the housekeeping promoters for α₁-ARs (Ramarao, C S., et al., J. Biol. Chem., 267:21936-21945 (1992)). However, the EGFP signal could be visualized without the use of an antibody if native tissue processing was done without the use of fixatives as shown for the α_{1B}-AR-EGFP mice (McGrath, personal communication) and for the interneuron recordings in this manuscript.

At birth, homozygous mice over-expressing either the α_{1A}-AR-GFP tagged receptor (designated α_{1A}-AR) or promoter-EGFP alone (designated α_{1A}-promoter) were viable and showed no gross phenotypic abnormalities. Reproductive capabilities were normal, unlike the α_{1B}-AR mice (Zuscik, M J, et al., Nat. Med., 6:1388-1394 (2000)). α_{1A}-AR mice also did not display any signs of neurodegeneration as assessed by histochemistry, abnormal gait, or the occurrence of seizures up to the age of 10 months unlike the α_{1B}-AR mice (Zuscik, M J, et al., Nat. Med., 6:1388-1394 (2000)). Receptor expression was verified and quantified through ligand binding in the brain, heart, kidney, and skeletal muscle and compared to normal non-transgenic mice. As shown in FIG. 1C, using the nonselective [I¹²⁵]-HEAT as a radiolabel which specifically labels total α₁-ARs and does not discriminate between the subtypes, increases in α₁-AR receptor density in the EGFP-tagged receptor mice was statistically different than normal mice or from promoter-only mice. Expression of the α_{1A}-AR was high in kidney, which is known to express α_{1A}-ARs but was absent in skeletal muscle, which do not express any α₁-AR subtype.

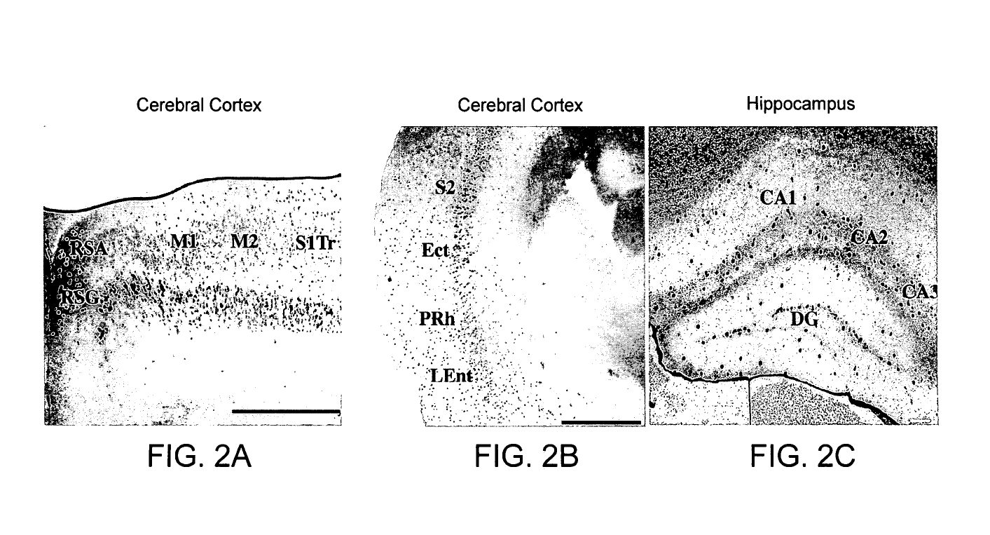

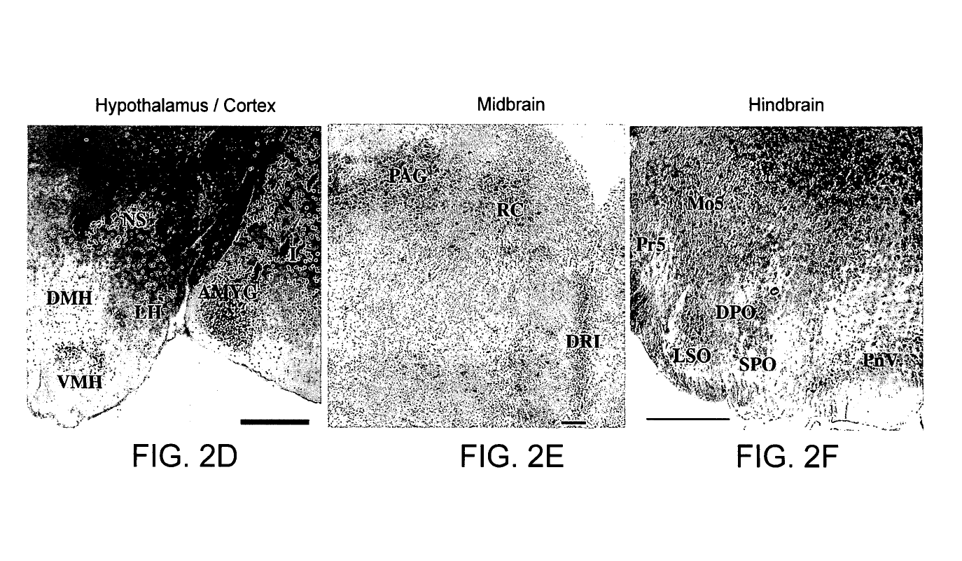

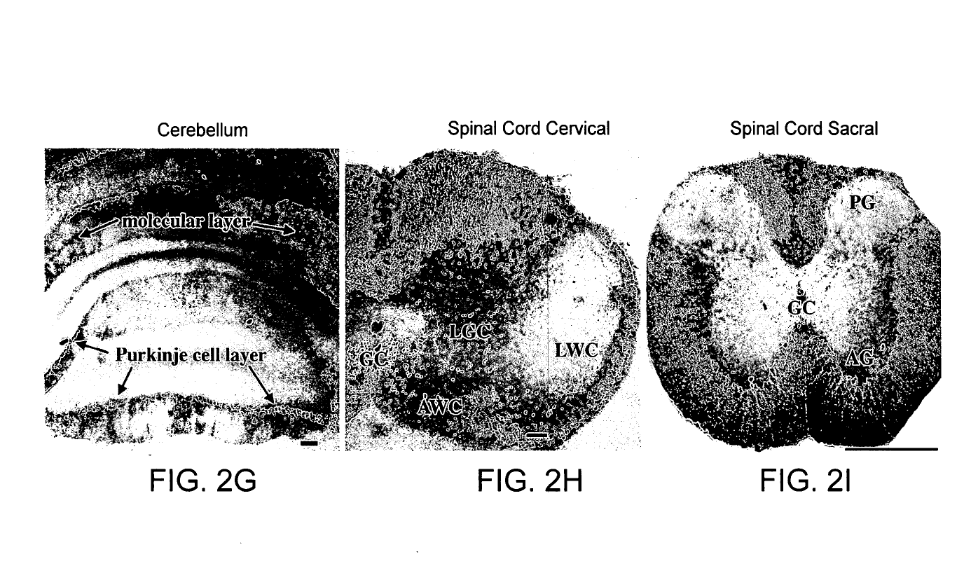

To assess whether the transgenic mice were expressing EGFP in the same manner as the endogenous receptor, an α_{1A}-AR KO mouse model which inserted the LacZ gene in place of the knocked out α_{1A}-AR first exon (Rokosh and Simpson, D G, et al., Proc. Natl. Acad. Sci., USA, 99:974-949 (2002)) was used. Using the X-gal substrate, which turns blue upon cleavage by the enzyme, β-galactosidase activity was assessed. This assessment was used to compare both mouse models at the protein level. Normal C57 mice, which are of the same genetic background as the α_{1A}-ARKO mice, were also used as a control and X-gal staining could not be detected in any sections. However, X-gal staining was found in the α_{1A}-ARKO mice in both a broad distribution but also in some layering in the cerebral cortex (FIG. 2A,B), the CA1-CA3 regions and dentate gyrus of the hippocampus (FIG. 2C), hypothalamic nuclei and the amygdala (FIG. 2D), midbrain (FIG. 2E), hindbrain (FIG. 2F), the purkinje and molecular layers of the cerebellum (FIG. 2G), the white and gray columns of the anterior and lateral cervical spinal cord (FIG. 2H), and the gray matter neuropil of the sacral spinal cord (FIG. 2I). All of the staining was found in cell bodies with some weak expression in the neuropil.

Immunohistochemistry was next performed on the EGFP mouse models to determine where the α_{1A}-AR was localized throughout the brain, as summarized in Table 2. Both mouse models were used (designated α_{1A}-promoter or α_{1A}-AR) and displayed similar patterns of expression. As controls, there was no detectable specific fluorescence in age-matched normal B6/CBA mice at the equivalent concentration of EGFP antibody. As previously reported (Papay, R., et al., J. Comp. Neurol., 478:1-10 (2004)), fixed mouse brain has high autofluorescence, which can be alleviated with copper sulfate treatment.

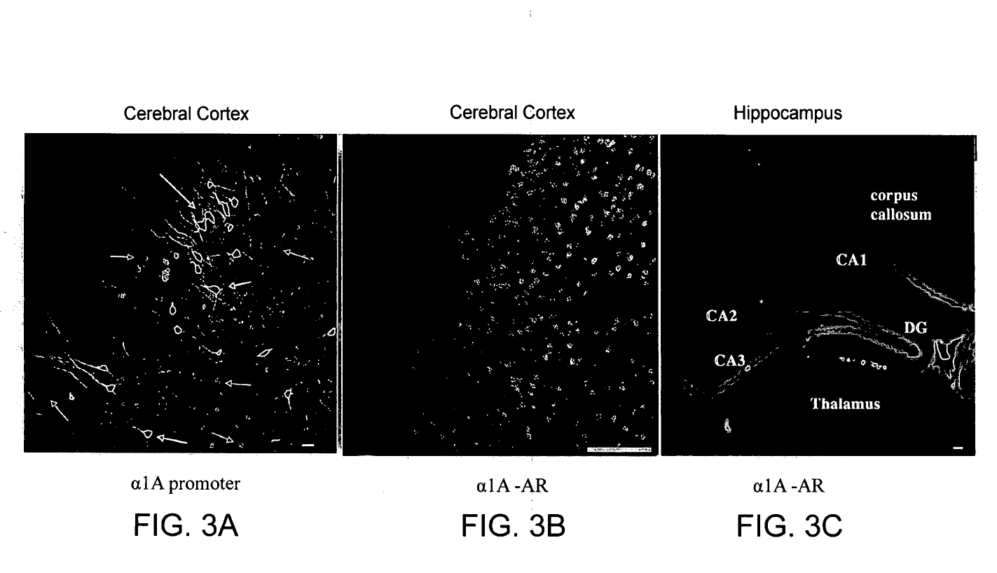

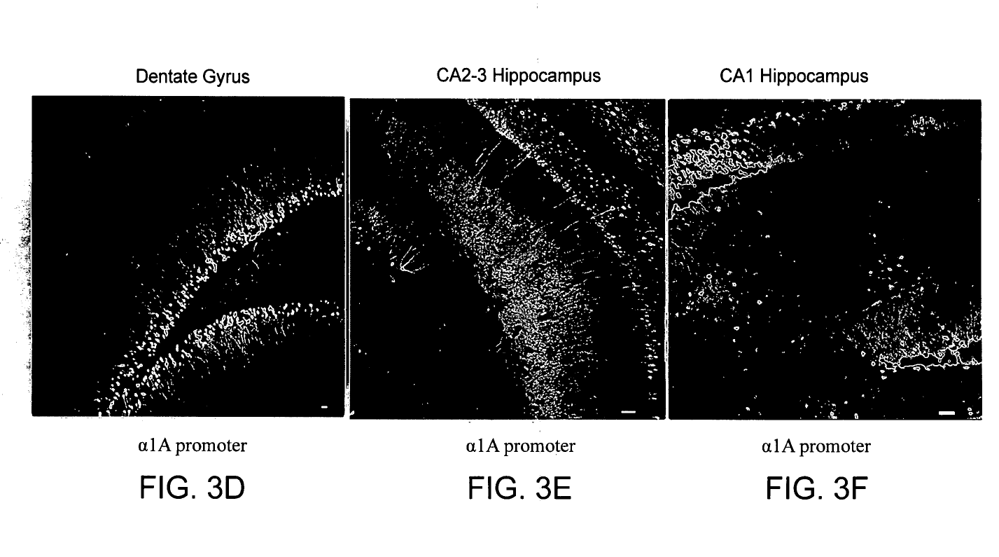

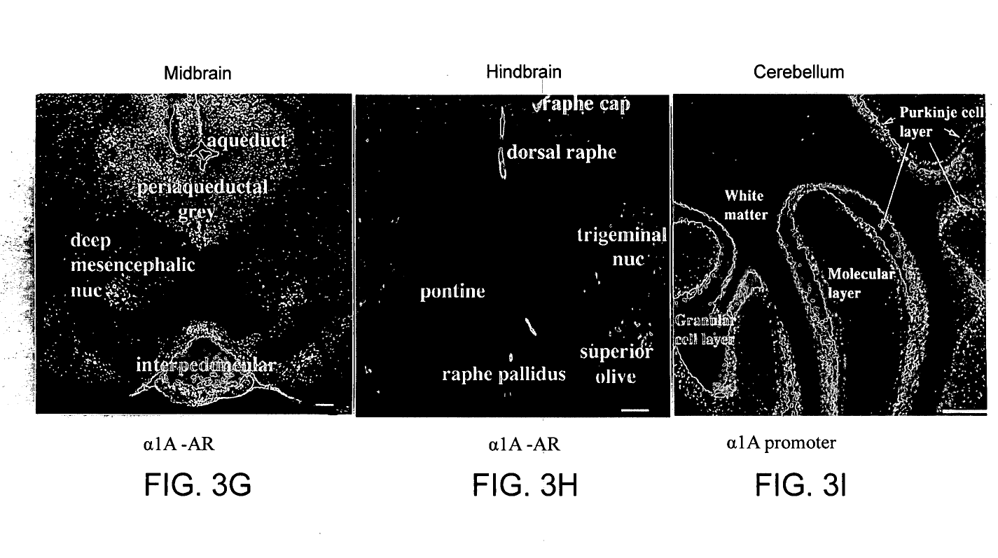

There was expression of EGFP in the cerebral cortex (FIG. 3A,B). While the α_{1A}-AR mouse model shows even distribution in the cerebral cortex (FIG. 3B), the α_{1A}-promoter mouse detects a similar distribution (red arrows, FIG. 3A) but also contained a cell type, which had greater expression than surrounding cells (white arrows, FIG. 3A). Expression of the EGFP was prominent in the cell body but in the case of the higher expressing cells (white arrows, FIG. 3A), expression was also present in the processes.

EGFP expression was prominent throughout the hippocampus (FIG. 3C) even under low magnification. Expression was intense in the granular cells of the dentate gyrus (FIG. 3D) but individual cells could be visualized in the various strata, such as the radiatum, oriens, pyramidal CA2-3 (FIG. 3E), and CA1 regions (FIG. 3F). Nuclei throughout the thalamus were weak. Expression was present in the hypothalamus/olfactory regions. There was also high expression of the α_{1A}-AR in the pituitary.

In the midbrain, the α_{1A}-AR was prominently expressed in the periaqueductal grey, interpeduncular nuclei, deep mesencephalic nuclei, and in cells that lined the aqueduct (FIG. 3G). In the hindbrain, expression was seen in the pontine, superior olive, trigeminal nuclei, and in the dorsal raphe and raphe cap (FIG. 3H). In the cerebellum, α_{1A}-AR expression was expressed in all layers of the cerebellum, but stronger in the Purkinje cell layers and totally absent in white matter tracts (FIG. 3I). Some autofluorescence was present in the granular layers, but EGFP was expressed over background.

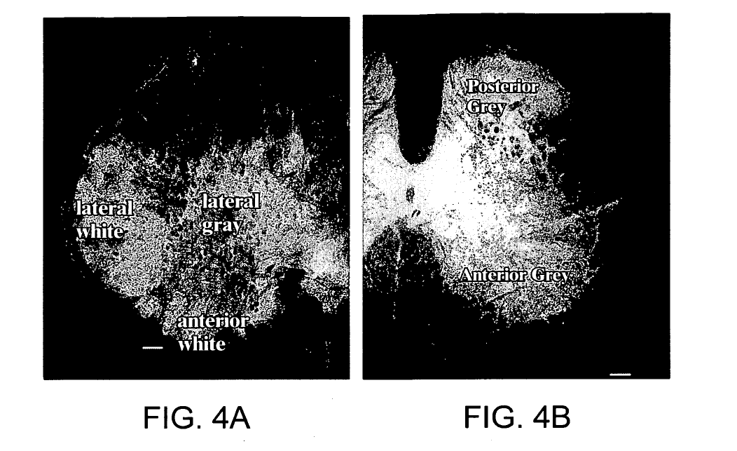

In the spinal cord, the α_{1A}-AR was located in the anterior and lateral white columns and the lateral and anterior grey columns in the upper cervical segments (FIG. 4A). Labeling appeared to be in the neuropil. In the deeper sacral segments, the α_{1A}-AR was still expressed in the lateral and anterior grey columns, but expression was gained in the posterior grey and lost in the lateral and anterior white columns (FIG. 4B).

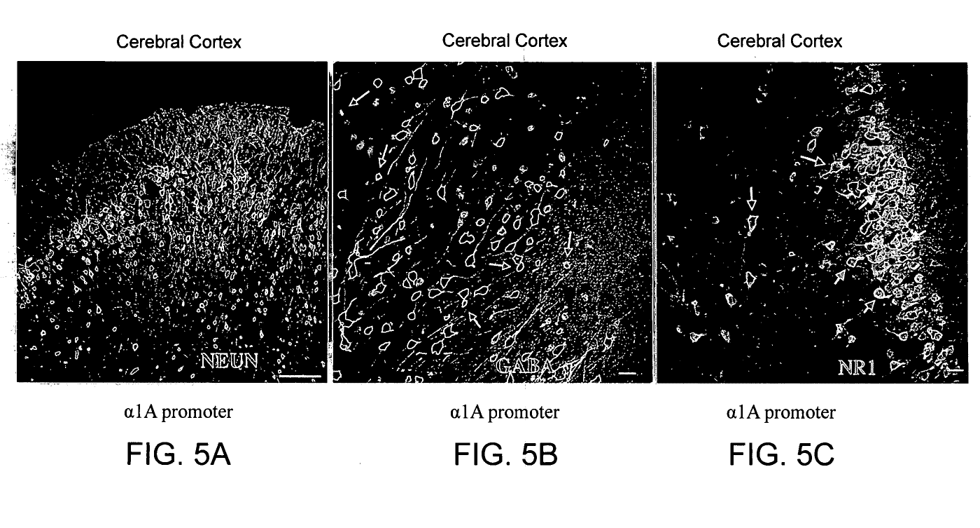

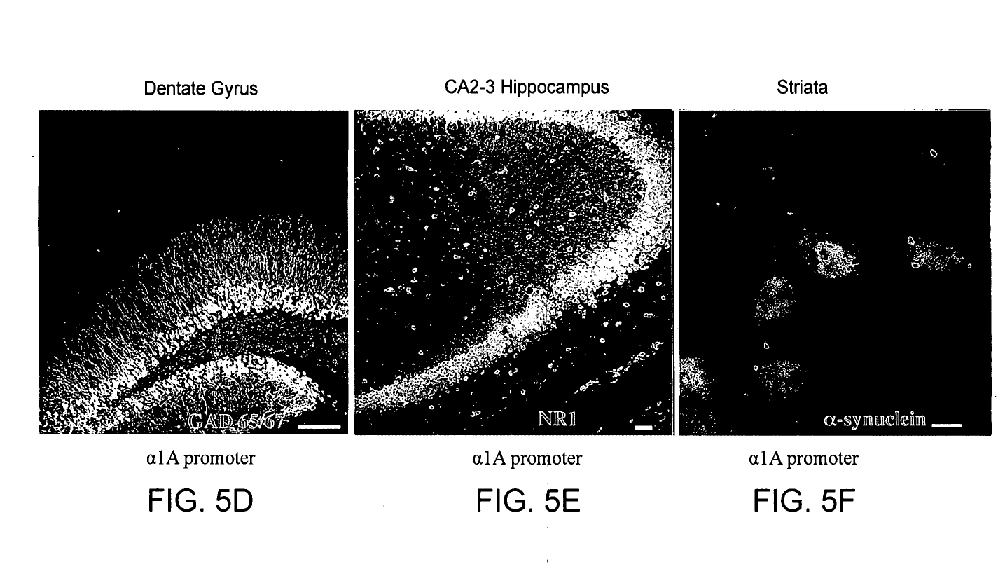

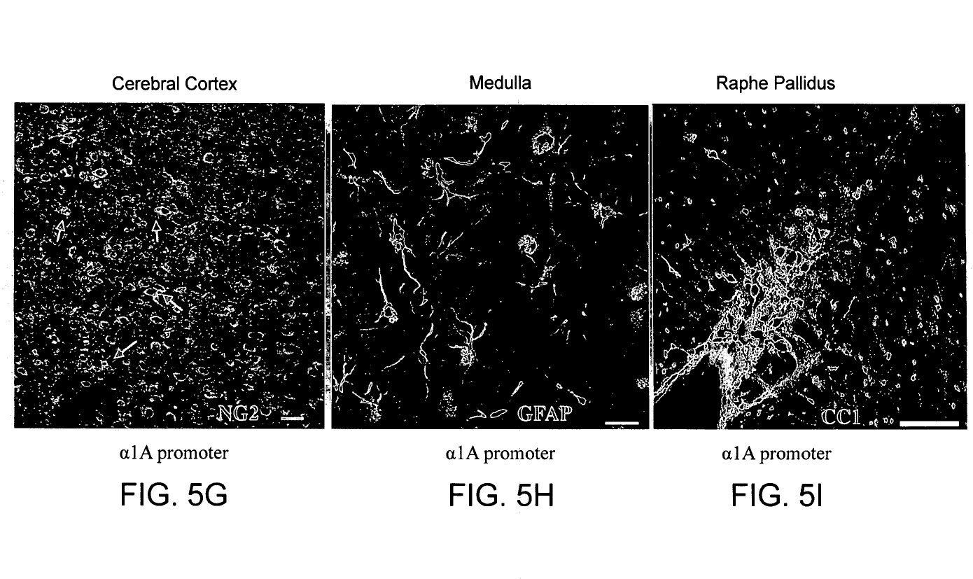

To determine what cell types expressed the α_{1A}-AR, neuronal cell types were the initial focus since by morphology this seemed to be the prominent cell expressing this subtype. This was confirmed by using an antibody against Neuronal Nuclei (NEUN) (FIG. 5A). NEUN coexpression with EGFP was evident throughout the brain.

Previous work has suggested that α₁-ARs can modulate GABA release in the human cerebral cortex (Ferraro, L., et al. Brain Res., 629:103-108 (1993)) and that α₁-ARs regulate CA1 interneurons in the rat hippocampus (Bergles, D E, et al., J. Neurosci., 16:572-585 (1996)). Work in the α_{1B}-AR transgenic mice suggests that GABA and NMDA receptor subunits, and especially the NR1 subunit undergo transcriptional regulation by _{1B}-ARS, confirmed through ligand binding and western blot experiments (Yun, J., et al., Brain, 126:2667-2681 (2003)). Antibodies against GABA and GAD were both used to assess GABAergic neurons for expression with EGFP. To confirm that α₁-ARs co-localized with these neurotransmitters and receptors, some EGFP-expressing neurons in the cerebral cortex co-localized with the neurotransmitter, GABA, (FIG. 5B), and with the NR1 subunit of the NMDA receptor (FIG. 5C). A population of EGFP-expressing cells in the granular layer of the dentate gyrus co-localized with GAD 65/67 (FIG. 5D) as well as cells in the CA2-3 hippocampus with the NR1 subunit (FIG. 5E). Antibodies against GABA labeled the cerebral cortex better than the hippocampus and GAD65/67 antibodies labeled the hippocampus better than the cerebral cortex.

While EGFP appears to be expressed in some neuronal processes, it did not appear to be expressed in nerve terminals. This was confirmed by using the presynaptic terminal protein α-synuclein, which did not co-localize with the EGFP signal in the striata (FIG. 5F). In analyzing other cell type markers, weak co-localization for the EGFP signal was found in the cerebral cortex with a marker for oligodendrocyte progenitors derived from the proteoglycan NG2 (FIG. 5G). However, the EGFP signal did not co-localize with the astrocyte or Bergmann glia marker, Glial Fibrillary Acidic Protein (GFAP) in the medulla (FIG. 5H) or in other parts of the brain. The EGFP signal also did not co-localize with a monoclonal antibody against adenomatous polyposis coli (APC; clone CC1), called CC1, which labels mature oligodendrocytes, in the region of the raphe pallidus nuclei (FIG. 5I), nor in other regions of the brain, or in white matter tracts. By morphology, the EGFP signal was not detected in cerebral blood vessels.

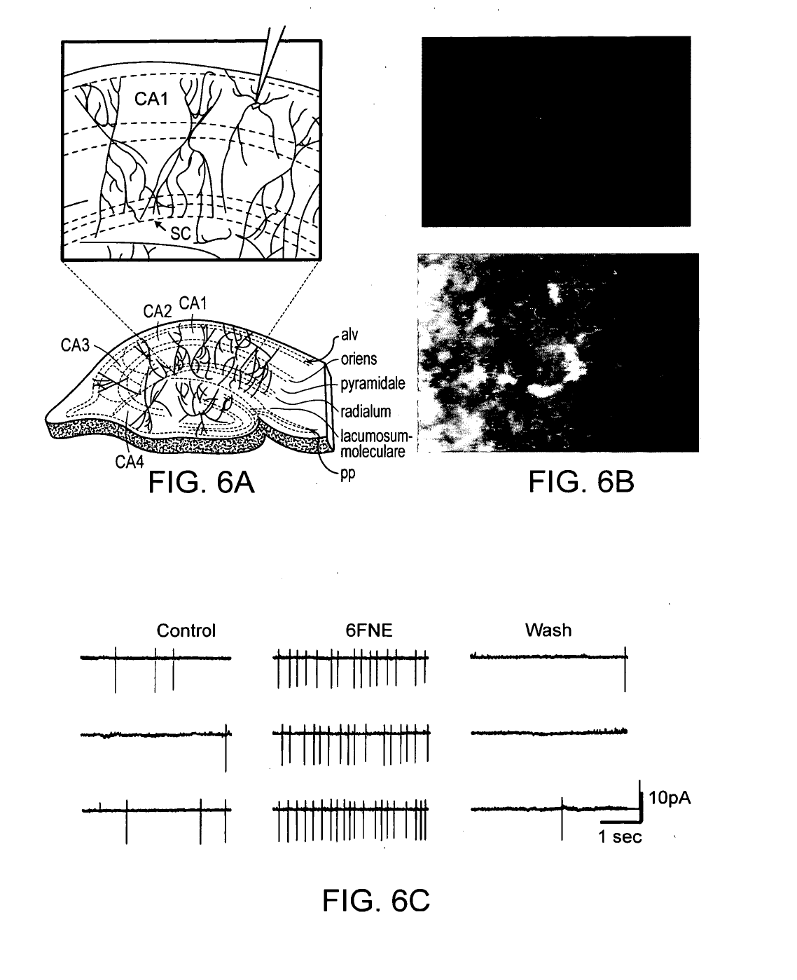

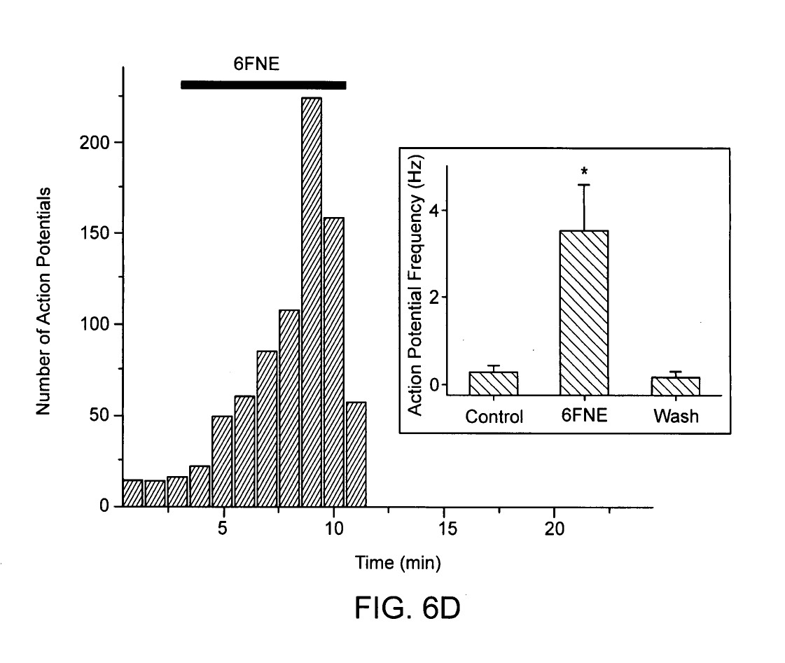

Using a combination of infrared imaging and electrophysiology techniques, EGFP-expressing cells in the stratum oriens were visualized (without antibody) and recorded in slices of mouse hippocampus. Multiple criteria (location, morphology and α-AR agonist response) were used to confirm that these EGFP-expressing cells were interneurons. α₁-AR-EGFP interneurons were initially identified by their location outside the distinct CA1 pyramidal cell layer (FIG. 6A). Unlike the tight stratification of pyramidal cell bodies, EGFP-expressing interneuron somata were scattered throughout all strata at low density (FIG. 6B). This is consistent with the distribution of GABA-immunoreactive cells in the hippocampal formation. Interneurons were also recognized by their heterogeneous, non-pyramidal morphology, which differed considerably from the highly uniform pyramidal shape observed for the CA1 principal cells. Cell-attached patch recordings from EGFP-expressing cells located in the stratum oriens of the hippocampal CA1 region (FIG. 6B) further confirmed that these cells were interneurons (FIGS. 6C, 6D). Consistent with the pharmacology of interneurons reported previously (Bergles, D E, et al., J. Neurosci., 16:572-585 (1996)), application of the selective α-AR agonist 6FNE (10 μM) caused a significant increase in the frequency of spontaneous action potential discharge compared with control conditions (mean 0.3±0.1 Hz, range 0-0.7 Hz, control; mean 3.5±1.1 Hz, range 1.8-6.5 Hz, 6FNE; n=4) (FIG. 6D Inset). Taken together, these results indicate that the α_{1A}-AR-EGFP expressing cells found outside the hippocampal CA1 pyramidal cell layer are interneurons.

### Discussion

The GPCR field is hindered by the lack of high avidity antibodies and highly selective ligands. Therefore, a series of systemically expressing transgenic mouse models whose expression is driven by their respective mouse α₁-AR promoters were developed to explore the localization of the α₁-AR subtypes in the mouse brain by tagging the receptors with an EGFP reporter (FIG. 1A). Fidelity of the mouse α_{1A}-AR promoter has been confirmed in endogenous and null cell lines by its regulation with various agents known to regulate these promoters (O'Connell, T D, et al., Mol. Pharmacol. 59:1225-1234 (2001); Rokosh and Simpson, Proc. Natl. Acad. Sci., USA, 99:9474-9479 (2002)). To confirm that the 4.4 kb promoter fragment confers all of the elements necessary for endogenous expression in the brain, β-galactosidase expression, engineered in the α_{1A}-AR KO mice to replace the α_{1A}-AR coding region, was compared to the transgenic mouse models and found to be similar (FIG. 2A-2I vs. FIG. 3A-3I, 4A-4B). Both models show prominent staining in neuronal cell bodies in the cerebral cortex, hypothalamus, hippocampus, midbrain, hindbrain, cerebellum and spinal cord and in similar areas. There are limitations with X-gal staining, in that although it was prominent it was weaker than fluorescence, cannot be used in confocal analysis, and it is diffusion-dependent, so outer layers may be more intensely stained than inner regions. This weaker inner staining may be true for the hippocampus (FIG. 2C), interior regions of the hypothalamus (FIG. 2D), and interior regions of the midbrain (FIG. 2E). Endogenous α_{1A}-AR distribution was also confirmed by ligand binding studies in α_{1A}-AR-positive or negative mouse tissues (FIG. 1C). The α_{1A}-AR-EGFP tissues expressed similar α₁-AR densities and distribution as the previous characterized CAM α_{1A}-AR in which expression was driven by the same promoter fragment (Rorabaugh, B R et al., Cardiovas. Res., 65:436-445 (2005)).

The distribution of α₁-AR subtypes in the brain has been previously examined by either autoradiography (Jones, L S, et al., J. Comp. Neurol., 231:190-208 (1985); Palacios, J M, et al., Brain Res., 419:65-75 (1987); Zilles, K, et al. Neurosci., 40:307-320 (1991)) or by in situ studies that utilize either oligonucleotide or cDNA probes (McCune, S K, et al., Neurosci., 57:143-151 (1993); Pieribone, V A, et al., J. Neurosci., 14:4252-4268 (1994); Nicholas, A P, et al., Ttrends Pharmacol. Sc., 17:245-255 (1996); Domyancic and Morilak, J. Comp. Neurol., 386.358-378 (1997)). Most of the studies have been reported in rat brain but they have been also analyzed in the mouse and they are similar if not identical to the rat (Palacios, J M, et al., Brain Res., 419:65-75 (1987)). Most autoradiography studies used a non-selective radiolabeled antagonist. While current technology makes in situ more resolving, these previous studies were not resolving in tissue to a cellular level and RNA can be transported from cell bodies. Therefore, previous studies only demonstrated global features of α₁-AR expression in the brain.

Early binding studies in membrane preparations have suggested that the α_{1A}-AR is predominate in the hippocampus and pons-medulla, while the α_{1B}-AR binding is highest in the thalamus and cerebral cortex (Wilson and Minneman, J. Neurochem., 53:1782-1786 (1989)). The α_{1B}-AR is also intense in the cerebral cortex by mRNA (Nicholas, A P, et al., Trends Pharmacol. Sc., 17:245-255 (1996)). Localization of the mRNA for the α_{1A}-AR was found in the hippocampus, cerebral cortex, and hindbrain but was most intense in a small region of the hippocampus, the dentate gyrus (Domyancic and Morilak, J. Comp. Neurol., 386:358-378 (1997)). In addition, mRNA localization did not provide enough resolution to see cells in the stratum layers. In agreement with these studies, it was found that the hippocampus was prominently expressing the α_{1A}-AR (FIG. 3C-F) and was much more intense than the cerebral cortex. However, there was even distribution within the various hippocampal layers and strata, as confirmed by the X-gal staining (FIG. 2C). Previous was weaker in comparison to the expression in the cerebral cortex (Papay, R., et al., J. Comp. Neurol., 478:1-10 (2004)). There was also high expression of the α_{1A}-AR in the hypothalamic region (fluorescence data not shown), also consistent with that previously seen for mRNA (Domyancic and Morilak, J. Comp. Neurol., 386:358-378 (1997)), and in the X-gal mice (FIG. 2D).

In the midbrain and hindbrain, many large cells throughout the mesencephalic nuclei, interpeduncular nuclei and the aqueductal/periaqueductal regions expressed the α_{1A}-AR (FIG. 2E, FIG. 3G). Nuclei in the trigeminal, pons, superior olive (FIG. 2F, FIG. 3H), as well as the raphe pallidus (FIG. 3H, FIG. 41) also expressed the α_{1A}-AR. These results are consistent with the localization of the mRNA for the α_{1A}-AR (Domyancic and Morilak, J. Comp. Neurol., 386:358-378 (1997)). In disagreement, the α_{1A}-AR was expressed in the dorsal raphe and raphe cap (FIG. 2E, FIG. 3H), areas previously thought to only express the α_{1B}-AR subtype (McCune, S K, et al., Neuroscience, 57:143-151 (1993); Pieribone, V A, et al., J. Neurosci., 14:4252-4268 (1994)).

The rat cerebellum was previously shown to contain moderate levels of the α_{1B}-AR by in situ studies with oligonucleotide probes (McCune et al., 1993; Pieribone et al., 1994). α_{1A}-AR mRNA did not localize to the rat cerebellum (Domyancic and Morilak, J. Comp. Neurol., 386:358-378 (1997)). However, binding studies in rat tissue indicate about half are α_{1A}-AR sites (Wilson and Minneman, J. Neurochem., 53:1782-1786 (1989)) and binding in the α_{1B}-AR knockout mouse suggests that the cerebellum has about 70% of non-α_{1B}-AR sites (Cavelli, A., et al., Proc. Natl. Acad. Sci., USA, 94:11589-11594 (1997)). There is little α_{1D}-AR in the mouse brain as determined in the α₁-AR knockout mouse (Tanoue, A., et al, J. Clin. Invest., 109:765-775 (2002)), and most of the α_{1D}-AR in the brain is likely to be localized in the paraventricular nucleus (Sands and Morilak, Neurosci., 91:639-649 (1999)). As shown herein, expression of the α_{1A}-AR subtype is prominent in the purkinje cell layers of the mouse cerebellum (FIG. 2G, FIG. 3I). These results are consistent with a roughly equal distribution of the two α₁-AR subtypes in mouse cerebellum, as assessed by binding studies. Since the mRNA for the α_{1A}-AR in the cerebellum did not localize (Domyancic and Morilak, J. Comp. Neurol., 386:358-378 (1997)) where the protein was found, this might indicate that the α_{1A}-AR mRNA may have been too little for detection.

Initial autoradiography studies using non-selective [³H]-prazosin in the rat spinal cord found that α₁-AR distribution was limited to only grey matter and there was equal distribution in the ventral and dorsal horns (Simmons and Jones, Brain Res., 445:338-349 (1988); Roudet et al., (1993)). In binding studies in rat tissues, the spinal cord was suggested to be mostly the α_{1A}-AR subtype, based upon insensitivity to chloroethylclonidine (Wilson and Minneman, J. Neurochem., 53:1782-1786 (1989)). Past studies using mRNA for the α_{1A}-AR (Domyancic and Morilak, J. Comp. Neurol., 386:358-378 (1997)) indicated localization of the α_{1A}-AR in the spinal cord ventral horns. As described herein, the α_{1A}-AR was localized to both the ventral and dorsal motor areas (FIG. 2 HI, FIG. 4AB) in agreement with Simmons and Jones, Brain Res., 445:338-349 (1988) and Roudet, C, et al., J. Neurosci. Res., 34:44-53 (1993), but was also localized to white matter columns in the cervical segments, which were not previously reported. The anterior grey matter controls skeletal and neuromuscular spindles inclusive of the limbs while the posterior grey matter is associated with sensing of position and movement. Both grey horns are consistent with the potential role of the α₁-ARs in movement control (Stone, E A, et al., Neuropharmacology, 40:254-261 (2001)). The anterior white columns control touch and pressure while the lateral columns control pain and thermal sensibilities. While rat spinal cord is suggested to be mostly composed of the α_{1A}-AR subtype (Wilson and Minneman, J. Neurochem., 53:1782-1786 (1989)), it was found that there were no differences in distribution or intensity between the α_{1A}- or _{1B}-AR subtypes in the mouse (Papay, R., et al., J. Comp. Neurol., 478:1-10 (2004)).

Of the neuronal cell types expressed in the hippocampus and cerebral cortex, it was found that the type of neuron expressing the α_{1A}-AR included the neurotransmitter GABA (FIGS. 5B, 5D) and the NR1 subunit of the NMDA receptor (FIGS. 5C, 5E). Also found was a similar distribution of GABA and NR1 containing neurons with the α_{1B}-AR-EGFP transgenic. Work in the α_{1B}-AR transgenic mice suggests that GABA and NMDA receptor subunits, and especially the NR1 subunit undergo transcriptional regulation by α_{1B}-ARs, confirmed through ligand binding and western blot experiments (Yun, J., et al., Brain, 126:2667-2681 (2003)). The neurotransmitter GABA mediates most of the inhibitory transmission events in the brain, while NMDA glutamate receptors mediate the vast majority of excitatory neurotransmission. Reported herein are that α₁-ARs co-localize with both GABAergic and NMDA receptor neurons and are likely to be involved in their regulation.

The major mechanism by which norepinephrine inhibits the excitability of pyramidal neurons may be an α₁-AR-mediated excitatory action on hippocampal CA1 interneurons (Bergles, D E, et al., J. Neurosci., 16:572-585 (1996)) and at piriform cortical interneurons (Marek and Aghajanian, Eur. J. Pharmacol., 305:95-100 (1996)). Electrophysiology studies showed that α_{1A}-AR-EGFP-expressing cells located outside the hippocampal pyramidal cell layer were clearly interneurons based on their location, morphology and response to α-AR stimulation. Interneurons comprise a heterogeneous group of non-pyramidal cells that utilize the inhibitory neurotransmitter GABA. In contrast to the highly stratified layer and uniform shape of pyramidal cells, α_{1A}-AR-EGFP-expressing cells were highly polymorphic and widely dispersed throughout all hippocampal strata, consistent with the distribution and morphology of GABA-immunoreactive cells in the hippocampal formation (Woodson, W., et al., J. Comp. Neurol., 280:254-271 (1989)). Hippocampal CA1 interneurons have also been previously shown to be highly responsive to α₁-AR activation by 6FNE (Bergles, D E, J. Neurosci., 16:572-585 (1996)), a highly selective α-AR agonist (Kirk, K L, et al., J. Med. Chem., 22:1493-1497 (1979)). In this study, action potential generation was significantly increased in α_{1A}-AR-EGFP-expressing cells treated with 6FNE, further demonstrating that these cells are interneurons. In contrast to its excitatory effects on interneurons, 6FNE inhibits action potential generation in pyramidal cells (unpublished data). The number of EGFP-expressing interneurons was lower in the α_{1B}-AR-EGFP mice compared to α_{1A}-AR-EGFP mice. Furthermore, the α_{1B}-AR-EGFP expressing interneurons appeared to be dying, consistent with seizure development in these mice (Kunieda, T., et al., Epilepsia, 43:1324-1329 (2002)). An α₁-AR-mediated facilitation of spontaneous GABA release from interneurons has been observed in several brain regions (Bergles, D E, et al., J. Neurosci., 16:572-585 (1996); Kawaguchi and Shindou, J. Neurosci., 18:6963-6976 (1998); Braga, M F., et al., Neuropsychopharmacology, 29:45-58 (2004)) and is believed to be one of the mechanisms underlying the antiepileptic properties of NE.

Oligodendrocyte progenitors can be identified using NG2 (Raff, M C., et al., Nature, 303:390-396 (1983)), a monoclonal antibody against a chondroitin sulfate proteoglycan that is selectively found in the early stages of oligodendrocyte development (Jones, L L., et al., J. Neurosci., 22:2792-2803 (2002)). NG2 positive cells are found in the adult mouse, are located in both white and grey matter, and comprise about 5-8% of the total number of cells in the brain (Dawson, M R., et al, J. Neurosci. Res., 61:471-479 (2000)). Recent evidence has suggested that two populations of NG2 progenitor cells exist (Mallon, B S, et al, J. Neurosci., 22:876-885 (2002)). One cell type will eventually differentiate into myelinating oligodendrocytes. The other population stays as the NG2 positive cells in the adult brain, proliferate, and may be involved in neurotransmission (Bergles, D E, et al., Nature, 405:187-191 (2000); Mallon, B S, et al., J. Neurosci., 22:876-885 (2002); Greenwood and Butt, Mol. Cell Neurosci., 23:544-558 (2003)). It was found that the α_{1A}-AR subtype is localized to NG2-oligodendrocyte progenitors (FIG. 5G) similar to the α_{1B}-AR-EGFP transgenic mouse (Papay, R., et al., J. Comp. Neurol., 478:1-10 (2004)).

A previous report found that all three α₁-AR subtypes are found in cultured progenitors and differentiated oligodendrocytes by PCR and using α1-AR antibodies, but only the α_{1A}-AR regulated inositol phosphate release (Khorchid, A., et al., Neuropharmacology, 42:685-696 (2002)). However, this data was not confirmed in tissue. As shown herein, in culture α_{1B}-AR EGFP positive cells mature into CC1-positive oligodendrocytes (Papay, R., et al., J. Comp. Neurol., 478:1-10 (2004)), but α₁-AR protein were not detected in mature oligodendrocytes in tissue (Papay, (2004)). It is possible that the α₁-AR protein is located in mature oligodendrocytes but cannot be detected in the system because of low abundance. It is likely that a 1-ARs are downregulated during the maturation process in vivo, but cannot be mimicked in vitro. Since neither α₁-AR subtype was found expressed in mature white matter tracts outside of the spinal cord, it is possible that the α₁-AR subtypes are involved in the developmental regulation of oligodendrocytes and be a switch in the maturation process.

The α_{1A}-AR, like the α_{1B}-AR, was not observed in cerebral blood vessels (Papay et al., 2004). The α₁-ARs play a major role in the constriction of peripheral blood vessels. While all three α₁-AR subtypes are found in peripheral blood vessels, it is believed that the α_{1A}-AR is the major subtype that regulates vasoconstriction (reviewed in Piascik and Perez, J. Pharmacol. Exp. Ther., 298:403-410 (2001)). However, this appears to not be the case in the cerebral vasculature. It has been suggested that the brain has evolved a mechanism in which sympathetic regulation does not constrict cerebral vascular tissue (Bevan, J A, et al., FASEB J., 1:193-198 (1987)), thereby preventing brain blood flow restriction during stress.

Most of the EGFP expression was found in the cell body, and sometimes was expressed in the processes by morphology. It did not appear to be expressed in cell terminals. To confirm whether the α_{1A}-AR is a postsynaptic or presynaptic receptor, an antibody against α-synuclein, which is an abundant protein that accumulates in presynaptic vesicles, was used (Iwai, A., et al., Neuron, 14:467-475 (1995); Clayton and George, Trends Neurosci., 21:249-254 (1998)). It was found that α-synuclein displayed a similar pericellular distribution in the mouse as previously reported (Ziolkowaks, B., et al., J. Neurosci., 25:4996-5003 (2005)) but did not co-localize with EGFP expression (FIG. 5F). This result is consistent with the previous assessment that the α₁-AR is a postsynaptic receptor (Arbilla and Langer, Br. J. Pharmacol., 64:259-264 (1978)).

In summary, described herein is the localization of the α_{1A}-AR in the CNS at the protein level using a transgenic EGFP-tagged approach that utilized a promoter fragment for the mouse α_{1A}-AR gene. The promoter fragment directed similar expression as LacZ, which was under the control of the endogenous promoter in the α_{1A}-knockout mouse. Compared to previous mRNA localization studies, the α_{1A}-AR EGFP expression was consistent with past studies in the cerebral cortex, hippocampus, hypothalamus, midbrain, hindbrain and grey areas of the spinal cord. Differences from past studies were observed in that EGFP cells could be detected in the various hippocampal strata in CA1-3, dorsal raphe, raphe cap, and white columns of the cervical spinal cord. Cerebellar localization of the α_{1A}-AR was entirely consistent with previous binding studies but not with mRNA in situ localization. The major cell-type that expressed the α_{1A}-AR was the neuron. The α_{1A}-AR is also expressed in interneurons in the stratum oriens, as well as GABAergic and NMDA receptor containing neuronal populations in the cerebral cortex and hippocampus. The α_{1A}-AR subtype is not expressed in cerebral blood vessels or mature white matter tracts except for the cervical spinal cord, similar to the α_{1B}-AR (Papay et al., 2004). The localization of the α_{1A} and α_{1B}-AR subtypes overlap in similar brain regions. However, the α_{1A}-AR was more abundant in the hippocampus, midbrain, and hindbrain than the α_{1B}-AR. The results described herein indicate that α_{1A}-ARs likely regulate the function of interneurons, and GABA/NMDA containing neurons, and are likely a switch to regulate the maturation of oligodendrocytes.

TABLE 2
Distribution of the α_{1A}- and α_{1B}-adrenergic receptors in the brain.
Plus and minus signs indicate various levels of relative expression.
α_{1B}-AR expression is shown for comparison (Papay et. al., 2004).
Differences between the two subtypes are bolded.

| Tissue | α_{1A}-AR | α_{1B}-AR |
|---|---|---|
| Accumbens nu | +++ | ++ |
| Amygdaloid | +++ | +++ |
| Basal Ganglia | | |
| Caudate/Put | + | + |
| Claustrum | + | + |
| Cerebellum | | |
| Granule cell layer | + | + |
| Molecular layer | + | + |
| Purkinje layer | +++ | + |
| Cerebral cortex | ++ | +++ |
| Piriform cortex | ++ | +++ |
| Corpus Callosum | − | − |
| Hindbrain | | |
| Cochlea | +++ | |
| Dorsal & ventral medullary reticular field | ++ | |
| Dorsal raphe nuc. | +++ | + |
| Medial vestibular nu | ++ | + |
| Parvocellularreticular nu | ++ | + |
| Pontine nuc. | +++ | +++ |
| Raphe pallidus | +++ | + |
| Reticulteg. nuc. pon | ++ | − |
| Rostral periolivary region | +++ | + |
| Spinal & sensory trigeminal nu | ++ | + |
| Superior olive | ++ | |
| Trigeminal Nuclei | ++ | |
| Ventral Spinocerebellar tract | +++ | ++ |
| Ventral tegmental nu | +++ | |
| Hippocampus | | |
| Dentate gyrus | +++ | + |
| CA1 | +++ | + |
| CA3 | +++ | + |
| Hypothalamus | | |
| Dorsomed. Ventromed. & lateral hypo. nu. | ++ | ++ |
| Preoptic | ++ | ++ |
| Peri & Paraventric. hypo. nu. | ++ | ++ |
| Interpeduncular nu | +++ | |
| Midbrain | | |
| Infer. Colliculus | ++ | ++ |
| Periaqueductal Grey | +++ | ++ |
| Raphe Cap | +++ | + |
| Raphe Pallidus nuclei | +++ | + |
| Substantia nigra | + | + |
| Super. Colliculus | + | ++ |
| Olfactory | +++ | ++ |
| Pituitary | ++ | ++ |
| Spinal Cord | ++ | ++ |
| Thalamus | + | + |

### The Following are Materials and Methods for Examples 2-8

### Materials and Methods

Neurosphere Isolation Periventricular tissue from 1-2 day old pups was removed into HEPES-buffered Eagles' medium. The tissue was diced and incubated with Mg/Ca-free HBSS containing EDTA, trypsin, and DNase. The tissue was washed, centrifuged, and the pellet was triturated in PBS to produce a single-cell suspension and was passed through a 70 um cell strainer. The cells were cultured in B27 media containing heparin, bFGF and EGF until neurosphere development. Neurospheres were dissociated and replated several passages. A single neurosphere was picked, mechanically dissociated, and cloned by dilution.

Differentiation Assay: The neurospheres were differentiated by withdrawing the growth factors either in the differentiation supplement (stem cell technologies) for embryonic neurospheres or in B-27 medium with 2% serum (for neonatal neurospheres) after dissociating them with the chemical dissociation kit (Stem Cell Technologies). The cells were plated at the density of 2×10⁵ cells in 12 well plates and treated with Phenylephrine (10 uM).

Ligand Binding. Membranes were prepared and ligand binding was performed as previously described (Rorabaugh et al., Cardiovas. Res., 65:436-445 (2005)). Saturation or competition binding used the nonselective i-AR antagonist, 2-[β-(4-hydroxyl-3-¹²⁵I-iodophenyl)ethylaminomethyl]-tetralone (¹²⁵I-HEAT) or ¹²⁵I-CYP as the radioligand, using either increasing concentration of the radiolabel or using a K_{D} amount (100 pM) to label total α₁-ARs and increasing concentrations of an non-labeled selective antagonist. Non-specific labeling was determined by adding 100 uM phentolamine (or α₁-ARs) or 1 uM propranolol (for β-ARs). Bₘₐₓ and K_{D} were determined through the use of Graphpad Prism. Statistical analysis of the binding data used a one-way ANOVA followed by a Newman-Keul's post-test where p<0.05 was considered significant.

Real Time PCR. Neurospheres were grown in proliferation or differentiation medium for 1, 3, 7 and 10 days respectively. The neurospheres grown in the proliferation medium were treated with phenlyephrine for the above-mentioned time along with Rauwalscine (0.1 uM) and Propranolol (1 uM). Total RNA was isolated using the RNAesy kit (Quiagen) and 2 ug RNA was reverse transcribed using oligodT primers with Superscript II Reverse Transcriptase (Invitrogen). Quantitative real time PCR was performed in iCycler (Biorad) using iQ SYBR Green Supermix. (Bio-Rad) The cDNA was amplified with primers for various neural stem cell proliferation and differentiation genes like Notch-1, Nestin, Mash-1, NeuroD, Math-1, Mash-1, Ngn-1, Ngn-2, D1x-2, Sox-2 and Zic-1. Samples obtained from three independent experiments were used for analysis of relative gene expression using the 2(-Delta Delta C(T) Method (Livak, K J and Schmittgen, T D, Methods 25:402-408 (2001)) and were normalized to α-Tubulin gene expression as internal control.

Proliferation studies: Dissociated neurospheres were seeded into culture dishes, grown in B27, bFGF and EGF media and cells were removed after 0, 1, 3, 7 in culture and counted after trypan blue exclusion.

Behavioral cognitive studies, Dry Maze: If the CAM α_{1A}-AR derived neurospheres suggest enhanced neurogenesis, this will likely affect cognitive functions and provide in vivo data. A dry maze test was performed on normal, CAM α_{1A}, CAM α_{1B}, and their corresponding KO models. The mice were trained 5 times per day on 5 consecutive days in a maze with the incorrect paths blocked, allowing only access to the correct solution. Along the correct path were visual clues. At the end of the maze, there was a peanut butter reward and a nylon mesh for escape. After training, each instance that the mice turned down the wrong path was counted as an error. Data were calculated as the time to complete the maze times the number of errors. On the fifth day, the mice were timed on their ability to solve the maze with all of the paths unblocked. This part of the experiment was considered a learning behavior.

Immunohistochemistry. Mice were injected with 0.1 ml heparin at 1000 U/ml i.p. and 0.1 ml Nembutal (50 mg/ml) i.p. Mice were perfused with 15 ml ice-cold heparin solution (10 U/ml) in PBS through the apex of the heart. This was followed by perfusion of 100 ml of 4% paraformaldehyde in phosphate buffer. The brain was removed and placed in 4% paraformaldehyde overnight at 4° C. The solution was replaced with cryoprotection solution (20% glycerol, 20% 0.4M Sorensen's Buffer, pH 7.6) overnight or longer at 4° C. Brains were embedded in 2-3% agarose in PBS after washing three times in PBS. 50-75 micron sections were cut using the Leica VT 1000 S vibrotome. Sections were put into cryoprotection solution (20% glycerol, 20% ethylene glycol, 60% PBS) and stored at 4° C. until ready for use.

Immunohistochemistry was performed in 24-well plates by a free-floating method. Specimens were first washed 3 times for 5 minutes in PBS, followed by incubation with blocking buffer (6% BSA+0.3% Trition X-100 in PBS) for 1 hour at room temperature on a shaker. The blocking buffer was removed and then replaced with the respective primary antibodies made up in blocking buffer for typically a 2 day incubation at 4° C. on a shaker. Next, the specimens were washed 3 times for 5 minutes in PBS, and then incubated for 1 hour at room temperature on a shaker with the respective secondary antibodies made up in blocking buffer. This was followed by 3 washes again for 5 minutes in PBS followed by two rinses in distilled water. The specimens were then incubated with 10 mM copper sulfate in 50 mM ammonium acetate for 1 hour at room temperature on a shaker to reduce autofluorescence (Schnell, Sa et al., J. Histochem. Cytochem., 47:719-730 (1999)). Finally, they were rinsed twice with distilled water and mounted with Vectashield Mounting Media with DAPI (Vector Laboratories, Burlingame, Calif., Cat. #H-1200).

Morris water maze. This consisted of a circular tub filled with water maintained at 26° C. to prevent hypothermia. In the middle was placed a stationary platform, just visible at the surface, which the mice can climb onto to be rescued from the water. Next to the stationary platform was a free-floating identical platform. The tub was also aligned with visual clues. The mice were placed at one end and were timed until they climbed onto the stationary platform. This procedure is repeated on day 1, 2, 5, and 6. This was considered the learning part of the test. Then the platforms and visual clues were reversed and the mice were retested. This was the spatial memory part of the test.

### Example 2

### Characterization of Mouse Neonatal Neurospheres Using a Cloning Assay

NSCs can be functionally defined by isolating and culturing cells so that they form clonal neurospheres (self-renew). To test the hypothesis that α₁-AR are expressed in NSCs, dissociated a i-AR containing mouse neurospheres should be able to regenerate neurospheres from a single cell and be able to differentiate into all three cell types upon α_{1A}-AR stimulation. Neurospheres were dissociated and diluted to a single cell level and distributed into 96-well plates. The percentage of single cells (% cloning efficiency) that regenerate neurospheres was then counted. As shown in FIG. 18A, neonatal neurospheres isolated from CAM α_{1A}-AR mice have a lower ability to regenerate neurospheres than normal or α_{1A}-KO mice, indicating that these neurospheres contain more differentiated progenitors than normal neurospheres. In addition, the α_{1A}-AR can influence the proliferation rate of isolated neonatal neurospheres. As shown in FIG. 18B, embryonic neurospheres from normal mice have the highest proliferation rates, followed by normal neonatal, and then CAM α_{1A}-AR, which had the slowest rate. The data indicate that α_{1A}-AR are expressed in NSCs and can influence their behavior to self-renew, and that the α_{1A}-AR likely induce the differentiation of NSCs.

### Example 3

### Direct Binding Experiment Showing Normal Mouse Neurospheres Contain α₁- and β-ARs

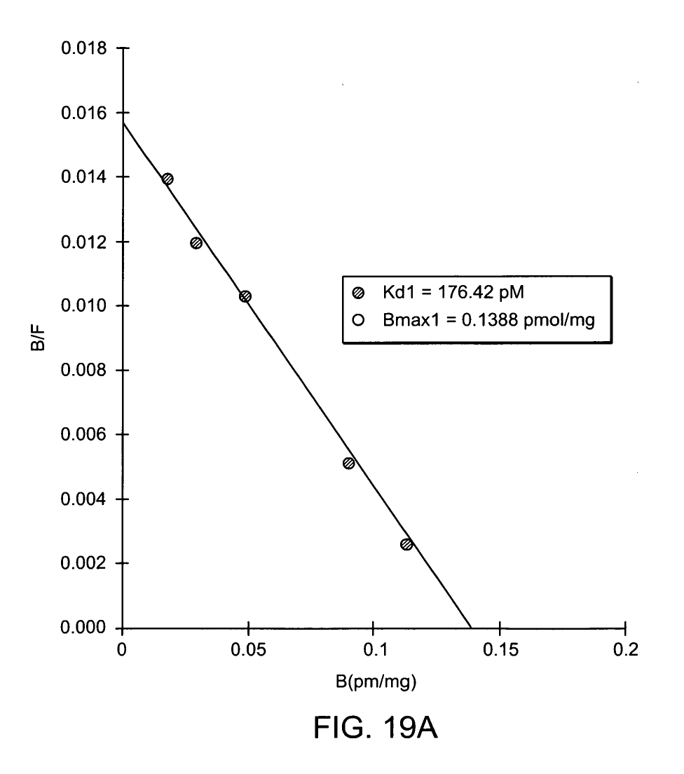

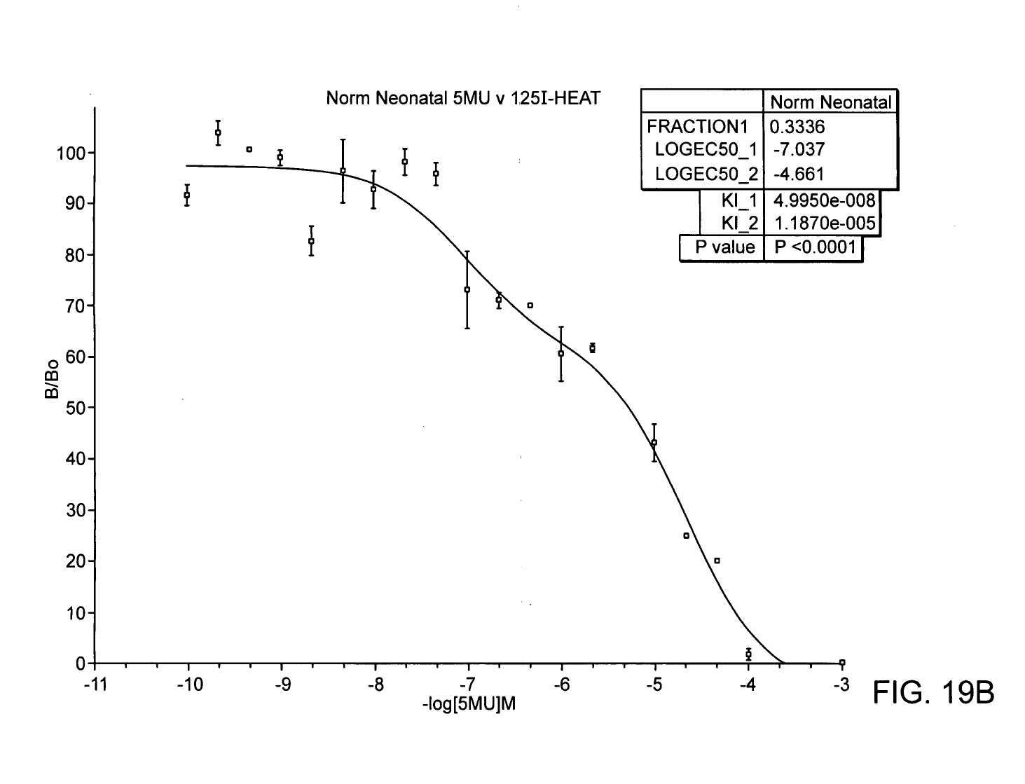

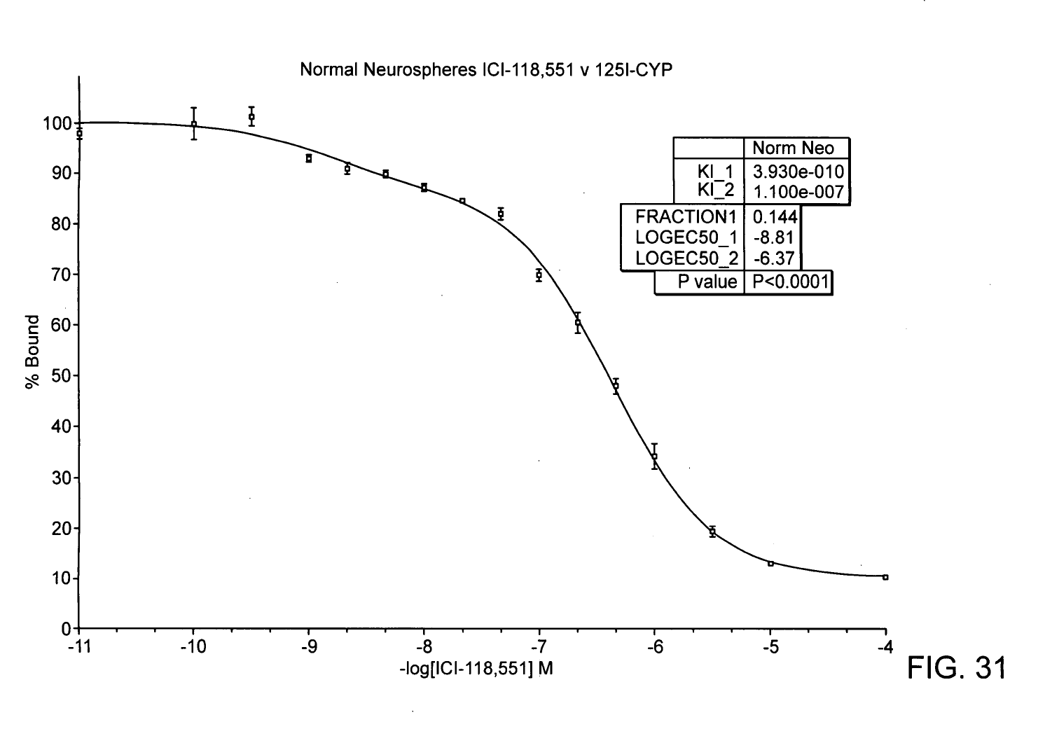

To confirm that normal mouse neurospheres contain α_{1A}-ARs, direct ligand binding experiments were performed. Saturation binding indicated that both normal embryonic and neonatal neurospheres express about 140 fmoles/mg protein with a Kd of 176 pM, results similar to other endogenous tissues (FIG. 19A). To reveal the α₁-AR subtype composition, competition ligand binding using 5-methylurapidil, which has high affinity for the α_{1A}-AR subtype but low affinity for the α_{1B}-AR subtype, was performed. Real time PCR has shown that mouse neurospheres do not express the α_{1D}-AR subtype. It was found that either embryonic or neonatal neurospheres express about 40% of the α_{1A}-AR subtype, leaving 60% as the α_{1B}-AR subtype (FIG. 19B). In addition, it was found that normal neurospheres express a dominance of the β₁-AR subtype (FIG. 31). The data indicates that a i- and β-ARs are functionally expressed on neurospheres and that the α_{1B}-AR as well as the α_{1A}-AR and β-ARs likely regulate their function.

### Example 4

### Characterization of Pluripotency

If the isolated neurospheres contain stem cells as indicated by the cloning assay of Example 2, and if the α_{1A}-ARs are expressed in NSCs, then each neurosphere culture from the various mouse models described herein should be able to produce the three types of cells in the brain when differentiated by serum (are pluripotent). As shown in FIG. 20A, normal neonatal neurospheres differentiated into all three cell types upon incubation with serum (2% fetal bovine serum (FBS)). However, CAM α_{1A}-AR neurospheres (FIG. 20B) also differentiated into all three cell types but they were mostly neurons (FIG. 20B, magenta, using the MAP2 marker) and NG2 oligodendrocytes (FIG. 20B, green, NG2 marker) with very little astrocytes (FIG. 20B, red, GFAP marker). These results are consistent with the results of Example 2 and indicate that the CAM α_{1A}-AR induced a higher number of differentiated neuronal and oligodendrocyte progenitors and has fewer undifferentiated stem cells.

As expected, α_{1A}-KO neurospheres (FIG. 20C) regained the ability to differentiate into astrocytes but had reduced levels of neurons and NG2 cells than normal cells, which is essentially the opposite phenotype of the CAM α_{1A}-AR neurospheres. The α_{1A}-KO data also indicates that the α_{1A}-AR is not essential but is required for the development of neurons and NG2 cells. If it was essential, there would have been no neurons or NG2 cells present in the α_{1A}-KO cells. However, there is also the possibility that the α_{1B}-AR, which is still expressed in the α_{1A}-KO neurospheres, also contributes to neuronal and NG2 development.

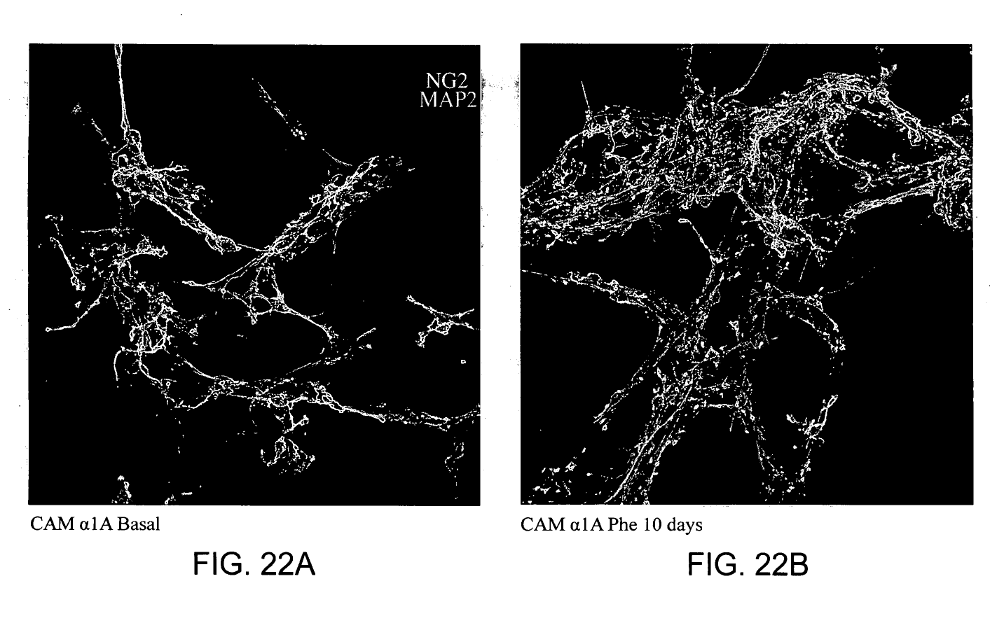

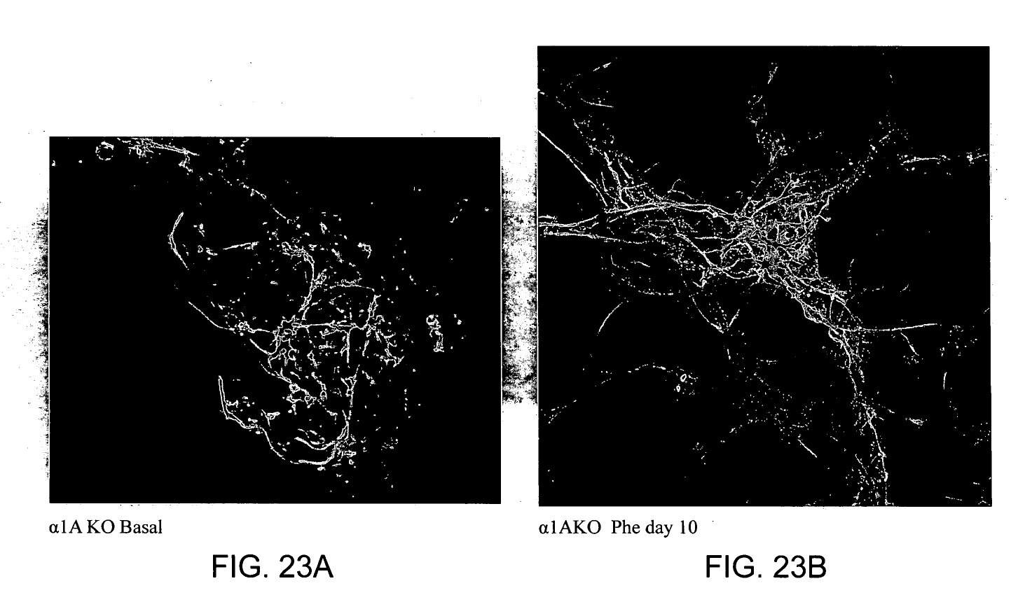

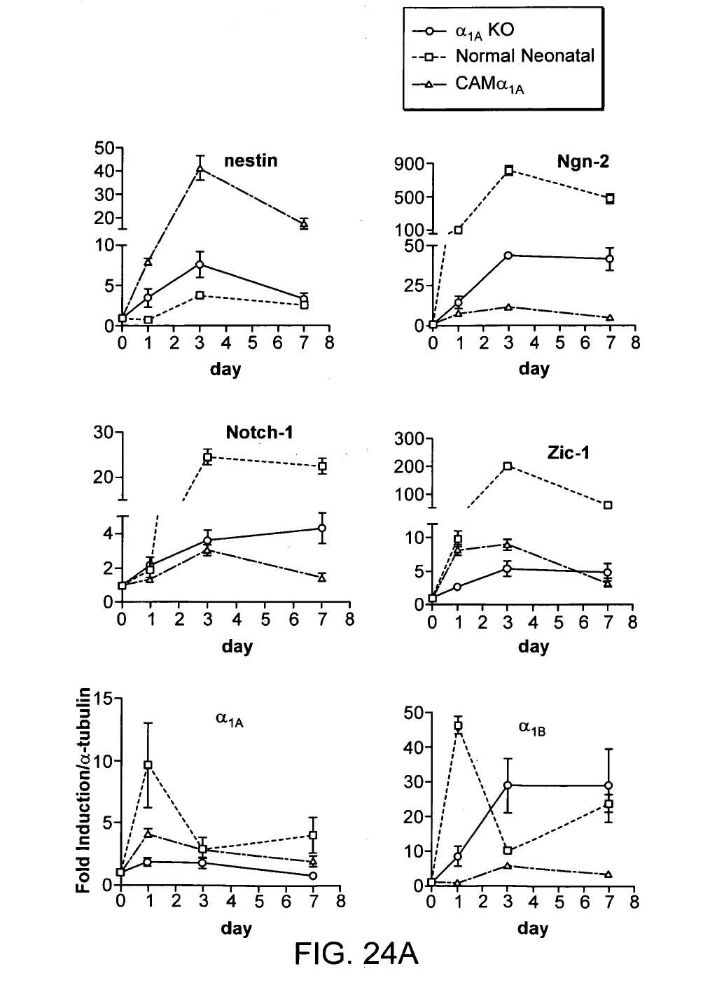

As additional proof that the α_{1A}-ARs are involved in neurogenesis, neurospheres should be able to differentiate into neuroblast and oligodendrocyte precursors upon prolonged phenylephrine (a i-AR agonist) stimulation. Phenylephrine (Phe) was added to a culture of normal neurospheres without withdrawing epidermal growth factor/fibroblast growth factor (EGF/FGF), which maintains the undifferentiated state, which expresses GFAP as shown in FIG. 21A. Phe induced differentiation into all three cell types (FIG. 21B), but upon longer stimulation preferred to form neuroblasts and NG2-positive cells (FIG. 21C). CAM α_{1A}-AR derived neurospheres are already differentiated (Basal, Day 0 stimulation) into MAP2-positive neuroblasts and NG2-positive oligodendrocytes with no or very little GFAP-positive astrocytes present (FIG. 22A). Phe has very little effect on the CAM α_{1A}-AR neurospheres because they are already differentiated (FIG. 22B). Again, KO of the α_{1A}-AR was opposite to the CAM α_{1A}-AR and restored GFAP expression (FIG. 23A) and stimulation with Phe increased formation of neurons (FIG. 23B), indicating that the α_{1B}-AR also plays a role in neurogenesis. Similar results were obtained for embryonic mouse neurospheres and adult neurospheres (1-2 months). Therefore, the results hold true for any age of stem cells. The results indicate that the α₁-ARs induce differentiation of neurospheres into neurons and oligodendrocytes and inhibit formation of astrocytes.

### Example 5

### α_{1A}-AR mRNA Regulation of Neurogenic Genes

Neurogenesis depends upon the interplay of endogenous genetic programs in addition to soluble factors and cellular interactions. If α_{1A}-ARs direct neuronal and NG2 oligodendrocyte precursor differentiation but inhibit astrocyte development, this should reflect in a specific pattern of gene expression regulation known to direct neurogenesis.

Real time polymerase chain reaction (PCR) studies were performed using the normal, CAM α_{1A}-AR and α_{1A}-KO neonatal neurospheres and RNA changes in genes known to play a role in neurogenesis during differentiation by serum for 0, 1, 3, 7 or 10 days of stimulation (FIG. 24). It was found that α_{1D}-AR was not expressed in neurospheres, which is not surprising since there is little α_{1D}-AR, if any, in the mouse brain (Tanoue, et al., J. Clin. Invest., 109:765-775 (2002)). Upon serum-induced differentiation, both the α_{1A}-AR and α_{1B}-AR subtypes increase mRNA expression in normal neurospheres (black lines), supporting the roles of these two subtypes in differentiation. This effect is abolished in the α_{1A}-KO for α_{1A}-AR subtype mRNA expression but enhances α_{1B}-AR mRNA expression, indicating that KO of one α₁-AR subtype has compensatory effects on the other subtype.

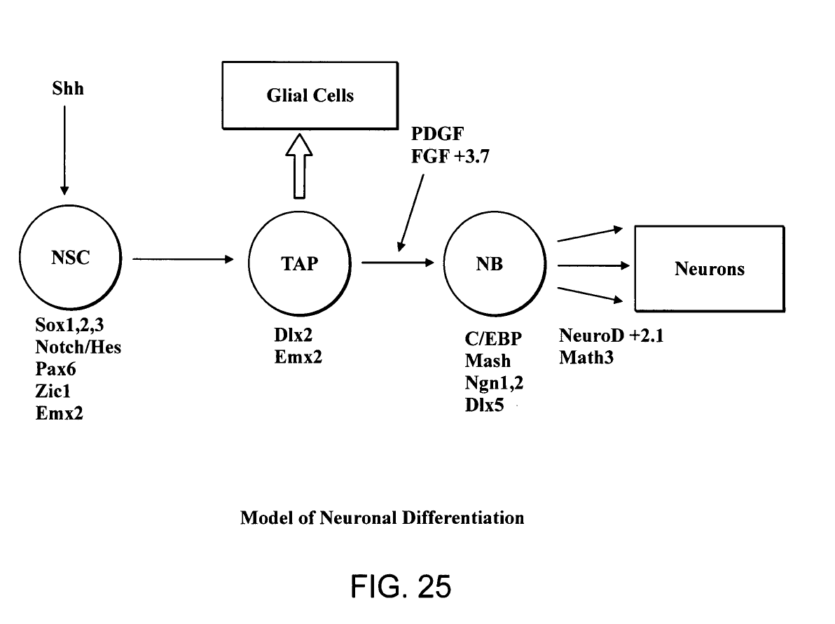

Consistent with the α_{1A}-AR regulating neuronal differentiation, the α_{1A}-KO reduced mRNA expression, compared with normal neurospheres (FIG. 24), of genes previously associated with neuronal differentiation, such as Ngn2, NeuroD, Math-1 and D1x2 (FIG. 25). The α_{1A}-KO also reduced expression of Notch-1, Zic-1 and Sox-2, which are associated with maintaining the undifferentiated state of neurospheres (FIG. 25). Again, gene expression was not abolished in the α_{1A}-KO, indicating that the α_{1B}-AR is also involved in neuronal differentiation of there is some additional regulation. The CAM α_{1A}-AR neurospheres had decreased expression for all genes except Nestin. An explanation for the decrease in expression in the CAM α_{1A} neurospheres is because they are already differentiated; therefore, these gene expressions cannot be further enhanced.

Similar data was obtained when phenylephrine (Phe) was used to differentiate the neurospheres, but expression levels were not to the same degree as serum differentiation (FIG. 26). The fold increase is much lower with Phe stimulation, except for expression of the α_{1A}-AR gene. Interestingly, Phe stimulation increased mRNA expression of the α_{1A}-AR by two hundred-fold, while α_{1B}-AR expression is increased only 10-fold. Therefore, there is likely some self regulation by the α_{1A}-AR. It is likely that a i-ARs, both α_{1A}-AR and α_{1B}-AR subtypes, can regulate expression of genes associated with neurogenesis and specifically with neuronal differentiation.

### Example 6

### Mechanism of Gene Expression

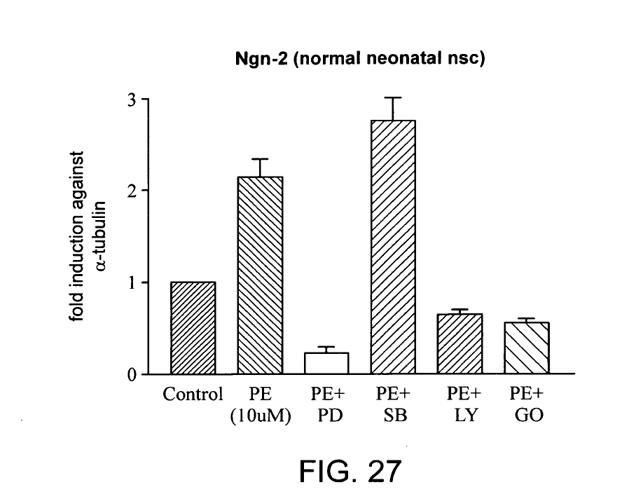

Since Ngn2 mRNA expression, a major gene of neuronal differentiation, appears to be regulated by the α_{1A}-ARs, the common a i-AR signals responsible for this gene regulation was determined. Real time PCR and inhibitors for PKC (GO6983), PI3K (LY294002), MEK (PD98059) and p38 (SB202190) were used on one day phenylephrine treated normal neonatal neurospheres. The α_{1A}-AR mediated increased Ngn2 expression was blocked by PKC, PI3K and MEK (ERK) inhibition but not by the p38 inhibitor (FIG. 27). This result indicates that α_{1A}-AR activation of the PI3K and ERK 1, 2 pathway(s) likely play a role in neurogenic gene expression. Since PI3K can confer apoptotic protection via the Akt pathway, these results indicated that the mechanism of neuronal differentiation by α_{1A}-ARs are through apoptotic cell death of astrocytes but protection from apoptosis for neurons and NG2 oligodendrocytes.

### Example 7

### Demonstration of Biological Function of α₁-ARs in Neurogenesis In Vivo and Positive Effects on Cognitive Behavior

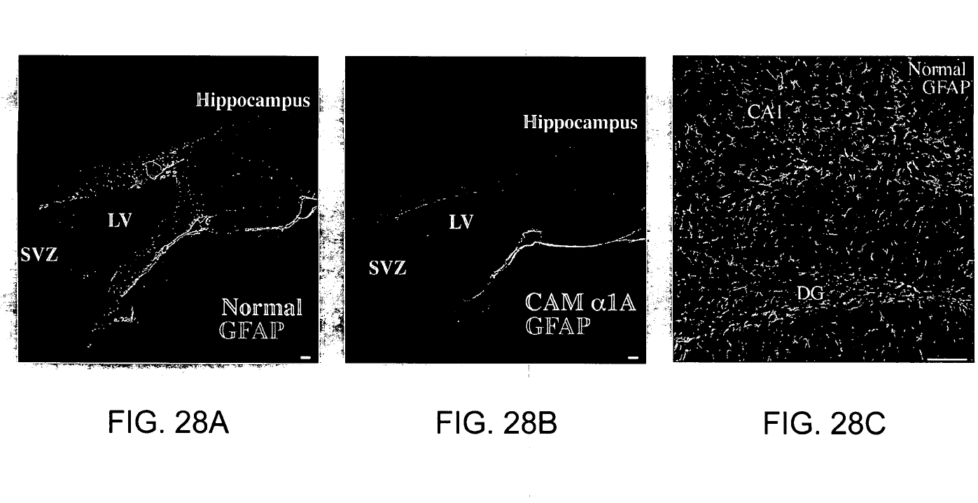

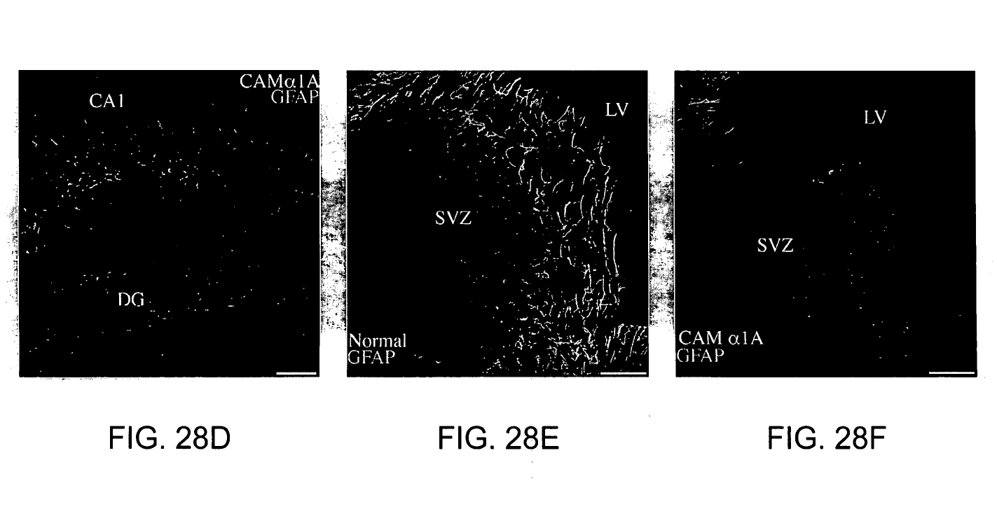

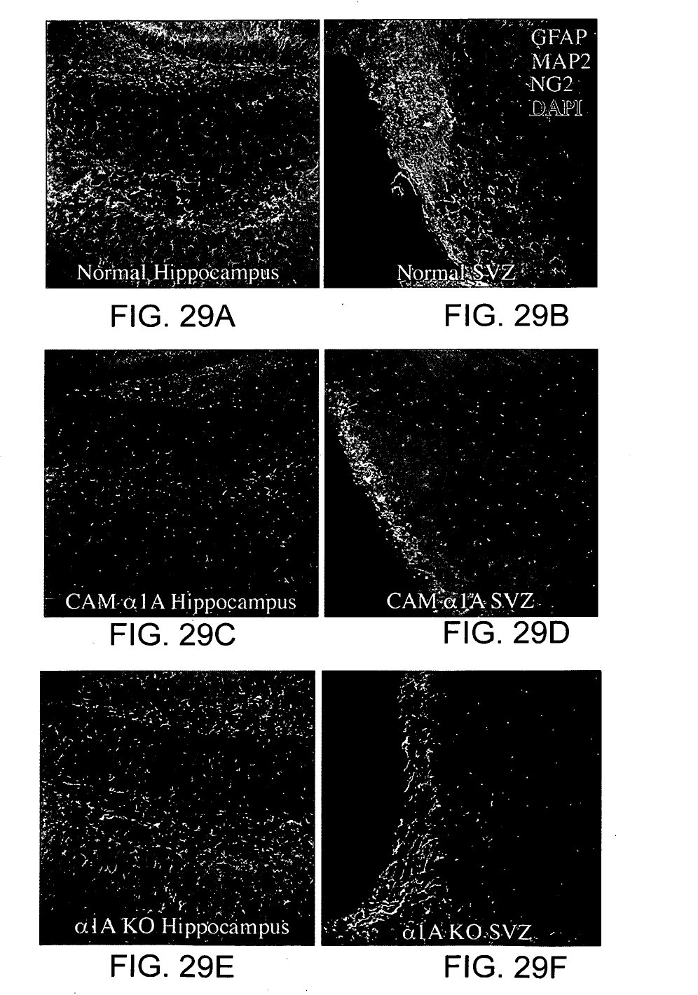

If the α₁-ARs regulate the neurogenic process resulting in the development of neurons and NG2⁺ oligodendrocytes, developmental changes are expected to take place consistent with that effect in the CAM and KO mice. Since CAM α_{1A}-AR derived neurospheres direct differentiation of only neuronal and NG2 oligodendrocyte precursors, it is expected that in vivo, the CAM α_{1A}-AR mice have developed less astrocytes, especially in the neurogenic regions. Processing both normal and the CAM α_{1A}-AR mouse sagittal vibrotome slices at the same time, using the same aliquot of antibody (GFAP to label astrocytes), and the same confocal parameters, it was demonstrated that the CAM α_{1A}-AR mice have a decreased abundance of astrocytes in the SVZ and hippocampal regions (FIGS. 28A-28F). Similar data was obtained when all three cell types were visualized (FIGS. 29A-29F). CAM α_{1A}-AR mice had much less astrocytes (red) in both the hippocampus (FIG. 29C) and SVZ (FIG. 29D) than normal mice (FIGS. 29A, 29B). α_{1A}-KO mice restored astrocyte expression but appeared to have less NG2 cells (green) and less neuronal markers (magenta) (FIGS. 29E, 29F). The results indicate that the α_{1A}-AR effects on differentiation seen in cultures translate to developmental changes in the adult in vivo.

### Example 8

### Cognitive Analysis

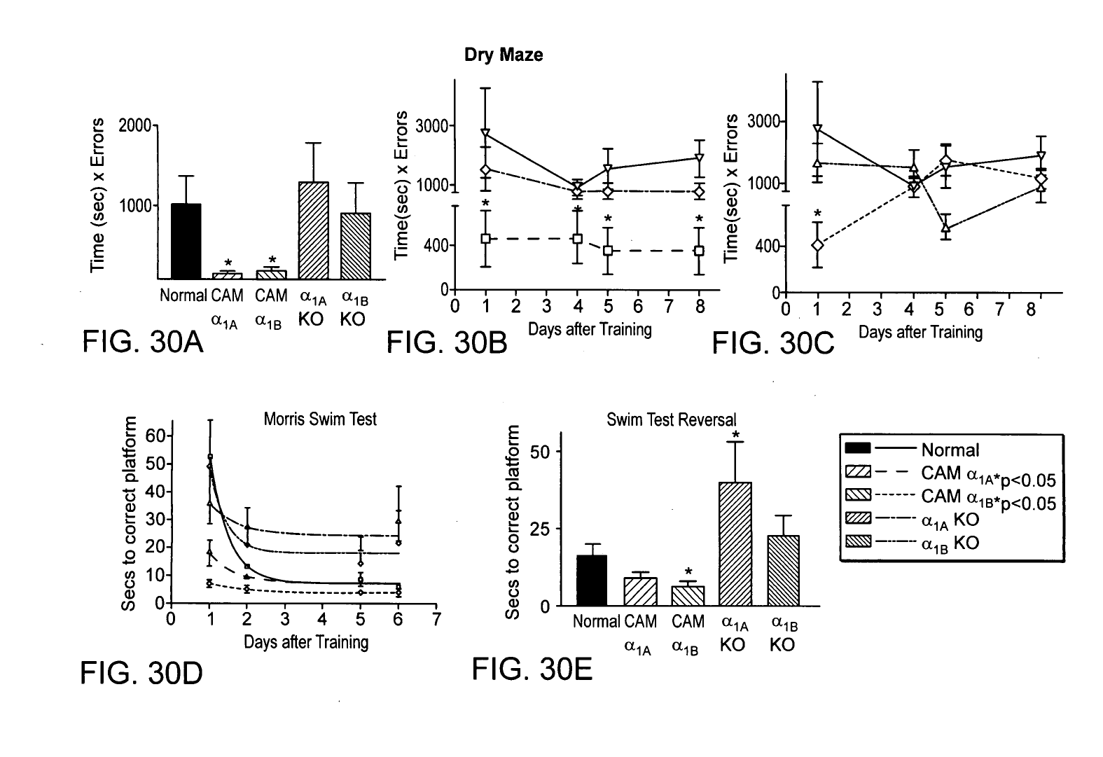

A dry maze test was performed on normal, CAM α_{1A}-AR, CAM α_{1B}-AR and their corresponding KO models. The mice were trained 5 times per day on 5 consecutive days in a maze with the incorrect paths blocked, allowing only access to the correct solution. Along the correct path were visual clues. At the end of the maze, there was a peanut butter reward and a nylon mesh for escape. On the fifth day of training, the mice were timed on their ability to solve the maze with all of the paths unblocked. Each instance that the mice turned down the wrong path was counted as an error. Data were calculated as the time to complete the maze times the number of errors. This part of the experiment was a learning behavior (FIG. 30A). It was determined that both the CAM α_{1A}-AR and CAM α_{1B}-AR had significantly enhanced learning behavior compared to normals or KOs. To test memory, the same mice were retested for 1, 4, 5 and 8 days after the initial training period (FIGS. 30B, 30C). It was found that only the CAM α_{1A}-AR mice retained the ability to solve the maze. The CAM α_{1B}-AR, while retaining the solution after 1 day, quickly lost its ability to solve the maze. The data indicate that both α_{1A}-AR and α_{1B}-AR are involved in learning but the α_{1A}-AR had better memory performance.

The Morris water maze test (Morris, R. J., et al., J Neurosci Methods, 11:47-60 (1984)), an additional test of cognitive function test, was performed on mice that were 2-3 months of age. A circular tub filled with water was maintained at 26° C. to prevent hypothermia. In the middle was placed a stationary platform just visible at the surface which the mice can climb onto to be rescued from the water. Next to the stationary platform was a free-floating identical platform. The tub was also aligned with visual clues. The mice were placed at one end and timed until they climbed onto the stationary platform. This procedure was repeated on days 1, 2, 5 and 6. This was the learning part of the test. The platforms and visual clues were reversed and the mice were retested. This was the memory part of the test. It was found that both the CAM α_{1A}-AR and CAM α_{1B}-AR performed better than normal or KO mice on the learning part of the swim test (FIG. 30D). When the platforms were reversed, the CAM α_{1B}-AR performed better than normal but the α_{1A}-KO was worse than normal (FIG. 30E).

These cognitive data indicate that a i-AR subtype activation enhances cognitive ability in memory and learning.

The teachings of all patents, published applications and references referred to herein are incorporated by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A transgenic non-human mammal whose genome comprises a recombinant nucleic acid sequence comprising an α_{1A}-adrenergic receptor (AR) and a marker peptide operably linked to all or a functional portion of an α_{1A}-AR promoter, wherein the α_{1A}-AR and the marker peptide are expressed as a fusion protein in the transgenic non-human mammal.

2. The transgenic non-human mammal of claim 1 wherein the non-human mammal is a rodent.

3. The transgenic mouse of claim 2 wherein the rodent is a mouse.

4. The transgenic non-human mammal of claim 1 wherein the recombinant nucleic acid sequence further comprises a promoter that directs expression of the fusion protein.

5. The transgenic non-human mammal of claim 4 wherein the promoter is a mouse α_{1A}-AR promoter.

6. The transgenic non-human mammal of claim 1 wherein the α_{1A}-AR is human α_{1A}-AR.

7. The transgenic non-human mammal of claim 1 wherein the marker peptide is a fluorescent peptide.

8. The transgenic non-human of claim 7 wherein the fluorescent peptide is selected from the group consisting of: a green fluorescent protein and an enhanced green fluorescent protein.

9. The transgenic non-human mammal of claim 1 wherein the marker peptide is fused to the N-terminus of the α_{1A}-AR.

10. The transgenic non-human mammal of claim 1 wherein the fusion protein is overexpressed in the non-human transgenic mammal.

11. A transgenic mouse whose genome comprises a recombinant nucleic acid sequence which comprises a mouse α_{1A}-adrenergic receptor (AR) promoter operably linked to a human α_{1A}-AR and an enhanced green fluorescent protein (EGFP), wherein the human α_{1A}-AR and the EGFP are expressed as a fusion protein in the transgenic mouse and the EGFP is fused to the C-terminus of the human α_{1A}-AR.

12. A transgenic non-human mammal whose genome comprises a recombinant nucleic acid sequence comprising a marker protein, under the control of all or a functional portion of an α_{1A}-AR promoter.

13. A method of producing a transgenic non-human mammal whose genome comprises a recombinant nucleic acid sequence comprising an α_{1A}-adrenergic receptor (AR) and a marker peptide, wherein the α_{1A}-AR and the marker peptide are expressed as a fusion protein in the transgenic non-human mammal, comprising:
a) introducing a targeting construct which comprises the recombinant nucleic acid sequence comprising the α_{1A}-AR and the marker peptide, wherein the α_{1A}-AR and the marker peptide are expressed as a fusion protein, into a pronuclei of an embryo;
b) introducing the embryo into a pseudo-pregnant non-human female mammal under conditions in which the non-human female mammal gives birth to a chimeric transgenic non-human mammal whose genome comprises the recombinant nucleic acid sequence comprising the α_{1A}-AR and the marker peptide;
c) breeding the chimeric transgenic non-human mammal with a second mammal to generate heterozygous F1 progeny that are heterozygous for the recombinant nucleic acid sequence comprising the α_{1A}-AR and the marker peptide; and
d) crossbreeding the heterozygous F1 progeny under conditions in which a transgenic non-human mammal whose genome comprises a recombinant nucleic acid sequence comprising an α_{1A}-adrenergic receptor (AR) and a marker peptide, wherein the α_{1A}-AR and the marker peptide are expressed as a fusion protein in the transgenic non-human mammal, and homozygote F2 progeny is produced.

14. A transgenic non-human mammal produced by the method of claim 13.

15. A targeting construct which comprises in a 5′ to 3′ direction about a 4.4 kb fragment of an α_{1A}-AR promoter sequence, an α_{1A}-AR sequence and an enhanced green fluorescent protein sequence.

16. An isolated cell or cell line whose genome comprises a recombinant nucleic acid sequence comprising an α_{1A}-adrenergic receptor (AR) and a marker peptide, wherein the α_{1A}-AR and the marker peptide are expressed as a fusion protein in the cell.

17. A method of identifying an agent that modulates α_{1A}-AR comprising:
a) administering the agent to a transgenic mouse or a cell isolate whose genome comprises a recombinant nucleic acid sequence which comprises α_{1A}-adrenergic receptor (AR) and a marker peptide, wherein the α_{1A}-AR and the marker peptide are expressed as a fusion protein in the transgenic mouse; and
b) determining whether α_{1A}-AR is modulated in the transgenic mouse or in the cell isolate compared to a control mouse or cell,
wherein if α_{1A}-AR is modulated in the transgenic mouse or cell isolate compared to the control mouse or cell, then the agent modulates α_{1A}-AR.

18. The method of claim 17 wherein whether α_{1A}-AR is modulated comprises determining whether the enhanced cognitive function associated with α_{1A}-AR is modulated.

19. A method of identifying an agent that modulates neural stem cell or progenitor cell differentiation or proliferation by α_{1A}-AR comprising:
a) administering the agent to a transgenic mouse or a cell isolate whose genome comprises a recombinant nucleic acid sequence which comprises α_{1A}-adrenergic receptor (AR) and a marker peptide, wherein the α_{1A}-AR and the marker peptide are expressed as a fusion protein in the transgenic mouse; and
b) determining whether expression of one or more neural stem cell markers is modulated in the transgenic mouse or in the cell isolate compared to a control mouse or cell,
wherein if the expression of the one or more neural stem cell marker is modulated in the transgenic mouse or cell isolate compared to the control mouse or cell, then the agent modulates neural stem cell or progenitor cell differentiation or proliferation by α_{1A}-AR.

20. A method of determining whether a cell is a neural stem cell comprising identifying markers expressed on the cell, wherein if the marker comprises α_{1A}-AR, nestin, notch 1, vimentin and glia fibrillary acidic protein (GFAP), then the cell is a neural stem cell.

21. A method of regulating differentiation or proliferation of a neural stem cell or progenitor cell comprising contacting the neural stem cell or progenitor cell with an agent that modulates biological activity of α_{1A}-AR, expression of α_{1A}-AR or a combination thereof, in the neural stem cell or progenitor cell.

22. The method of claim 21 wherein the differentiation or proliferation of the neural stem cell or progenitor cell is enhanced comprising contacting the neural stem cell or progenitor cell with an agent that enhances biological activity of α_{1A}-AR, expression of α_{1A}-AR or a combination thereof, in the neural stem cell or progenitor cell.

23. The method of claim 22 wherein the neural stem cell differentiates into one or more cells selected from the group consisting of: a transiently amplifying progenitor (TAP) cell, a neuroblast, an oligodendrocyte and a combination thereof.

24. The method of claim 21 wherein the differentiation of the neural stem cell or progenitor cell is inhibited comprising contacting the neural stem cell or progenitor cell with an agent that inhibits biological activity of α_{1A}-AR, expression of α_{1A}-AR or a combination thereof, in the neural stem cell or progenitor cell.

25. The method of claim 21 further comprising contacting the neural stem cell or progenitor cell with an agent that modulates biological activity of α_{1B}-AR, expression of α_{1B}-AR, biological activity of β-AR, expression of β-AR or a combination thereof, in the neural stem cell or progenitor cell.

26. A method of treating a neurodegenerative disorder in an individual in need thereof, comprising administering to the individual an agent that regulates biological activity of α_{1A}-AR, expression of α_{1A}-AR or a combination thereof, in the individual.

27. The method of claim 26 wherein the neurodegenerative disorder is selected from the group consisting of: Alzheimer's Disease, Parkinson's Disease, Multiple System Atrophy and spinal cord injuries.

28. A method of enhancing cognitive function in an individual in need thereof comprising administering to the individual an agent that enhances biological activity of an α₁-AR, expression of an α₁-AR or a combination thereof, in the individual.

29. The method of claim 28 wherein the α₁-AR is selected from the group consisting of: α_{1A}-AR, α_{1B}-AR, β-AR and a combination thereof.

30. The method of claim 28 wherein the cognitive function is selected from the group consisting of: learning, memory and a combination thereof.
